(19) United States
(12) Patent Application Publication
(10) Pub. No.: US 2025/0051447 A1
(43) Pub. Date: Feb. 13, 2025

(54) FULLY CANINE ANTI-CANINE PD-1 ANTIBODIES AND USES THEREOF

(71) Applicant: The Trustees of the University of Pennsylvania, Philadelphia, PA (US)

(72) Inventors: Donald L. Siegel, Lansdale, PA (US); Nicola J. Mason, Philadelphia, PA (US); Nicholas Chester; Keren Paz, Tenafly, NJ (US)

(21) Appl. No.: 18/718,387

(22) PCT Filed: Dec. 9, 2022
(86) PCT No.: PCT/US2022/081297

§ 371 Date: Jun. 10, 2024

Related U.S. Application Data
(60) Provisional application No. 63/288,284, filed on Dec. 10, 2021.

(51) Int. Cl. C07K 16/28

(52) U.S. Cl. CPC C07K 16/2818; C07K 2317/20; C07K 2317/622; C07K 2317/76

(57) ABSTRACT

The present disclosure relates to canine antibodies, binding polypeptides, scFvs specific for canine programmed death protein 1 (PD-1), nucleic acids encoding the same, and the methods to prepare the same and to use the same to treat a disease or a condition.

### CROSS-REFERENCE TO RELATED APPLICATION

The present application claims priority under 35 U.S.C. § 119 (e) to U.S. Provisional Patent Application No. 63/288,284, filed Dec. 10, 2021, which is hereby incorporated by reference in its entirety herein.

### BACKGROUND

The immune checkpoint inhibitors (ICI) pembrolizumab and nivolumab have revolutionized the treatment of patients with different cancers including melanoma, renal cell carcinoma, and non-small cell lung carcinoma. Such ICI therapies work by blocking receptors that negatively regulate the function of cytotoxic T cells. In effect, ICIs act to block signaling pathways that would otherwise restrain T cell function. While immune checkpoint receptors normally play a key role in preventing immune responses from becoming toxic, many cancers take advantage of these negative signals to blunt T-cell immune responses directed against them, often by expressing ligands for immune checkpoint receptors expressed on cytotoxic T cells.

Immune competent dogs develop spontaneous tumors that exhibit features such as chromosome aberrations, molecular subtypes, immune signatures, tumor heterogeneity, metastatic behavior, and response to chemotherapy. However, with the current absence of fully canine, commercially available anti-canine PD-1 (cPD-1) antibodies in the veterinary arena, the potential clinical benefits of checkpoint inhibition in treating canine cancer patients cannot be realized.

There is a need in the art for the development of non-immunogenic canine antibodies against immune checkpoint molecules that can be used to treat veterinary cancers in canine subjects through the blockade of PD-1 on tumor-specific T cells.

### SUMMARY

In one aspect, disclosed herein is an antibody or antigen-binding fragment thereof that specifically binds to canine programmed death protein 1 (cPD-1), wherein the antibody or antigen-binding fragment thereof comprises: a heavy chain complementarity determining region 1 (HCDR1), comprising the amino acid sequence set forth in SEQ ID NOs: 1, 27, or 43, a heavy chain complementarity determining region 2 (HCDR2), comprising the amino acid sequence set forth in SEQ ID NOs: 2, 28, or 44, a heavy chain complementarity determining region 3 (HCDR3), comprising the amino acid sequence set forth in SEQ ID NOs: 3, 29, or 45, a light chain complementarity determining region 1 (LCDR1), comprising the amino acid sequence set forth in SEQ ID NOs: 4, 30, or 46, a light chain complementarity determining region 2 (LCDR2), comprising the amino acid sequence set forth in SEQ ID NOs: 5, 31, or 47, and a light chain complementarity determining region 3 (LCDR3), comprising the amino acid sequence set forth in SEQ ID NOs: 6, 32, or 48.

In some embodiments, the antibody or antigen-binding fragment thereof comprises: a heavy chain variable region comprising an amino acid sequence having at least 80%, 85%, 90%, 95%, 96%, 96%, 97%, 98%, 99% identity to the amino acid sequence set forth in SEQ ID NOs: 7, 9, 33, or 49; and a light chain variable region comprising an amino acid sequence having at least 80%, 85%, 90%, 95%, 96%, 97%, 98%, 99% identity to the amino acid sequence set forth in SEQ ID NOs: 11, 35, or 51.

In some embodiments, the antibody or antigen-binding fragment thereof comprises: an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, 23, 39, or 55, and an immunoglobulin light chain, comprising an amino acid sequence having at least 80%, 85%, 90%, 95%, 96%, 97%, 98%, 99% identity to the amino acid sequence set forth in SEQ ID NOs: 25, 41, or 57.

In one aspect, disclosed herein is an antibody or antigen-binding fragment thereof that specifically binds to canine programmed death protein 1 (cPD-1), wherein the antibody or antigen-binding fragment thereof comprises: a heavy chain variable region comprising an amino acid sequence having at least 80%, 85%, 90%, 95%, 96%, 96%, 97%, 98%, 99% identity to the amino acid sequence set forth in SEQ ID NOs: 7, 9, 33, or 49; and a light chain variable region comprising an amino acid sequence having at least 80%, 85%, 90%, 95%, 96%, 97%, 98%, 99% identity to the amino acid sequence set forth in SEQ ID NOs: 11, 35, or 51.

In one aspect, disclosed herein is an antibody or antigen-binding fragment thereof that specifically binds to canine programmed death protein 1 (cPD-1), wherein the antibody or antigen-binding fragment thereof comprises: a heavy chain variable region comprising the amino acid sequence set forth in SEQ ID NOs: 7, 9, 33, or 49; and a light chain variable region comprising the amino acid sequence set forth in SEQ ID NOs: 11, 35, or 51.

In one aspect, disclosed herein is an antibody or antigen-binding fragment thereof that specifically binds to canine programmed death protein 1 (cPD-1), wherein the antibody or antigen-binding fragment thereof comprises: an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, 23, 39, or 55, and an immunoglobulin light chain, comprising an amino acid sequence having at least 80%, 85%, 90%, 95%, 96%, 97%, 98%, 99% identity to the amino acid sequence set forth in SEQ ID NOs: 25, 41, or 57.

In one aspect, disclosed herein is an antibody or antigen-binding fragment thereof that specifically binds to canine programmed death protein 1 (cPD-1), wherein the antibody or antigen-binding fragment thereof comprises: an immunoglobulin heavy chain, comprising the amino acid sequence set forth in SEQ ID NOs: 19, 21, 23, 39, or 55, and an immunoglobulin light chain, comprising the amino acid sequence set forth in SEQ ID NOs: 25, 41, or 57.

In one aspect, disclosed herein is an antibody or antigen-binding fragment thereof that specifically binds to canine programmed death protein 1 (cPD-1), wherein the antibody or antigen-binding fragment thereof comprises: a heavy chain complementarity determining region 1 (HCDR1), comprising the amino acid sequence set forth in SEQ ID NO: 1, a heavy chain complementarity determining region 2 (HCDR2), comprising the amino acid sequence set forth in SEQ ID NO: 2, a heavy chain complementarity determining region 3 (HCDR3), comprising the amino acid sequence set forth in SEQ ID NO: 3, a light chain complementarity determining region 1 (LCDR1), comprising the amino acid sequence set forth in SEQ ID NO: 4, a light chain complementarity determining region 2 (LCDR2), comprising the amino acid sequence set forth in SEQ ID NO: 5, and a light chain complementarity determining region 3 (LCDR3), comprising the amino acid sequence set forth in SEQ ID NO: 6.

In some embodiments, the antibody or antigen-binding fragment thereof comprises: a heavy chain variable region comprising an amino acid sequence having at least 80%, 85%, 90%, 95%, 96%, 96%, 97%, 98%, 99% identity to the amino acid sequence set forth in SEQ ID NOs: 7 or 9; and a light chain variable region comprising an amino acid sequence having at least 80%, 85%, 90%, 95%, 96%, 97%, 98%, 99% identity to the amino acid sequence set forth in SEQ ID NO: 11.

In some embodiments, the antibody or antigen-binding fragment thereof comprises: an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, or 23, and an immunoglobulin light chain, comprising an amino acid sequence having at least 80%, 85%, 90%, 95%, 96%, 97%, 98%, 99% identity to the amino acid sequence set forth in SEQ ID NO: 25.

In one aspect, disclosed herein is an antibody or antigen-binding fragment thereof that specifically binds to canine programmed death protein 1 (cPD-1), wherein the antibody or antigen-binding fragment thereof comprises: a heavy chain variable region comprising an amino acid sequence having at least 80%, 85%, 90%, 95%, 96%, 96%, 97%, 98%, 99% identity to the amino acid sequence set forth in SEQ ID NOs: 7 or 9; and a light chain variable region comprising an amino acid sequence having at least 80%, 85%, 90%, 95%, 96%, 97%, 98%, 99% identity to the amino acid sequence set forth in SEQ ID NO: 11.

In one aspect, disclosed herein is an antibody or antigen-binding fragment thereof that specifically binds to canine programmed death protein 1 (cPD-1), wherein the antibody or antigen-binding fragment thereof comprises: a heavy chain variable region comprising the amino acid sequence set forth in SEQ ID NO: 7; and a light chain variable region comprising the amino acid sequence set forth in SEQ ID NO: 11.

In one aspect, disclosed herein is an antibody or antigen-binding fragment thereof that specifically binds to canine programmed death protein 1 (cPD-1), wherein the antibody or antigen-binding fragment thereof comprises: a heavy chain variable region comprising the amino acid sequence set forth in SEQ ID NO: 9; and a light chain variable region comprising the amino acid sequence set forth in SEQ ID NO: 11.

In one aspect, disclosed herein is an antibody or antigen-binding fragment thereof that specifically binds to canine programmed death protein 1 (cPD-1), wherein the antibody or antigen-binding fragment thereof comprises: an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, or 23, and an immunoglobulin light chain, comprising an amino acid sequence having at least 80%, 85%, 90%, 95%, 96%, 97%, 98%, 99% identity to the amino acid sequence set forth in SEQ ID NO: 25.

In one aspect, disclosed herein is an antibody or antigen-binding fragment thereof that specifically binds to canine programmed death protein 1 (cPD-1), wherein the antibody or antigen-binding fragment thereof comprises: an immunoglobulin heavy chain, comprising the amino acid sequence set forth in SEQ ID NOs: 19, and an immunoglobulin light chain, comprising the amino acid sequence set forth in SEQ ID NO: 25.

In one aspect, disclosed herein is an antibody or antigen-binding fragment thereof that specifically binds to canine programmed death protein 1 (cPD-1), wherein the antibody or antigen-binding fragment thereof comprises: an immunoglobulin heavy chain, comprising the amino acid sequence set forth in SEQ ID NOs: 21, and an immunoglobulin light chain, comprising the amino acid sequence set forth in SEQ ID NO: 25.

In one aspect, disclosed herein is an antibody or antigen-binding fragment thereof that specifically binds to canine programmed death protein 1 (cPD-1), wherein the antibody or antigen-binding fragment thereof comprises: an immunoglobulin heavy chain, comprising the amino acid sequence set forth in SEQ ID NOs: 23, and an immunoglobulin light chain, comprising the amino acid sequence set forth in SEQ ID NO: 25.

In one aspect, disclosed herein is an antibody or antigen-binding fragment thereof that specifically binds to canine programmed death protein 1 (cPD-1), wherein the antibody or antigen-binding fragment thereof comprises: a heavy chain complementarity determining region 1 (HCDR1), comprising the amino acid sequence set forth in SEQ ID NO: 27, a heavy chain complementarity determining region 2 (HCDR2), comprising the amino acid sequence set forth in SEQ ID NO: 28, a heavy chain complementarity determining region 3 (HCDR3), comprising the amino acid sequence set forth in SEQ ID NO: 29, a light chain complementarity determining region 1 (LCDR1), comprising the amino acid sequence set forth in SEQ ID NO: 30, a light chain complementarity determining region 2 (LCDR2), comprising the amino acid sequence set forth in SEQ ID NO: 31, and a light chain complementarity determining region 3 (LCDR3), comprising the amino acid sequence set forth in SEQ ID NO: 32.

In some embodiments, the antibody or antigen-binding fragment thereof comprises: a heavy chain variable region comprising an amino acid sequence having at least 80%, 85%, 90%, 95%, 96%, 96%, 97%, 98%, 99% identity to the amino acid sequence set forth in SEQ ID NO: 33; and a light chain variable region comprising an amino acid sequence having at least 80%, 85%, 90%, 95%, 96%, 97%, 98%, 99% identity to the amino acid sequence set forth in SEQ ID NO: 35.

In some embodiments, the antibody or antigen-binding fragment thereof comprises: an immunoglobulin heavy chain, comprising an amino acid sequence having at least 80%, 85%, 90%, 95%, 96%, 97%, 98%, 99% identity to the amino acid sequence set forth in SEQ ID NO: 39, and an immunoglobulin light chain, comprising an amino acid sequence having at least 80%, 85%, 90%, 95%, 96%, 97%, 98%, 99% identity to the amino acid sequence set forth in SEQ ID NO: 41.

In one aspect, disclosed herein is an antibody or antigen-binding fragment thereof that specifically binds to canine programmed death protein 1 (cPD-1), wherein the antibody or antigen-binding fragment thereof comprises: a heavy chain variable region comprising an amino acid sequence having at least 80%, 85%, 90%, 95%, 96%, 96%, 97%, 98%, 99% identity to the amino acid sequence set forth in SEQ ID NO: 33; and a light chain variable region comprising an amino acid sequence having at least 80%, 85%, 90%, 95%, 96%, 97%, 98%, 99% identity to the amino acid sequence set forth in SEQ ID NO: 35.

In one aspect, disclosed herein is an antibody or antigen-binding fragment thereof that specifically binds to canine programmed death protein 1 (cPD-1), wherein the antibody or antigen-binding fragment thereof comprises: a heavy chain variable region comprising the amino acid sequence set forth in SEQ ID NO: 33; and a light chain variable region comprising the amino acid sequence set forth in SEQ ID NO: 35.

In one aspect, disclosed herein is an antibody or antigen-binding fragment thereof that specifically binds to canine programmed death protein 1 (cPD-1), wherein the antibody or antigen-binding fragment thereof comprises: an immunoglobulin heavy chain, comprising an amino acid sequence having at least 80%, 85%, 90%, 95%, 96%, 97%, 98%, 99% identity to the amino acid sequence set forth in SEQ ID NO: 39, and an immunoglobulin light chain, comprising an amino acid sequence having at least 80%, 85%, 90%, 95%, 96%, 97%, 98%, 99% identity to the amino acid sequence set forth in SEQ ID NO: 41.

In one aspect, disclosed herein is an antibody or antigen-binding fragment thereof that specifically binds to canine programmed death protein 1 (cPD-1), wherein the antibody or antigen-binding fragment thereof comprises: an immunoglobulin heavy chain, comprising the amino acid sequence set forth in SEQ ID NO: 39, and an immunoglobulin light chain, comprising the amino acid sequence set forth in SEQ ID NO: 41.

In one aspect, disclosed herein is an antibody or antigen-binding fragment thereof that specifically binds to canine programmed death protein 1 (cPD-1), wherein the antibody or antigen-binding fragment thereof comprises: a heavy chain complementarity determining region 1 (HCDR1), comprising the amino acid sequence set forth in SEQ ID NO: 43, a heavy chain complementarity determining region 2 (HCDR2), comprising the amino acid sequence set forth in SEQ ID NO: 44, a heavy chain complementarity determining region 3 (HCDR3), comprising the amino acid sequence set forth in SEQ ID NO: 45, a light chain complementarity determining region 1 (LCDR1), comprising the amino acid sequence set forth in SEQ ID NO: 46, a light chain complementarity determining region 2 (LCDR2), comprising the amino acid sequence set forth in SEQ ID NO: 47, and a light chain complementarity determining region 3 (LCDR3), comprising the amino acid sequence set forth in SEQ ID NO: 48.

In some embodiments, the antibody or antigen-binding fragment thereof comprises: a heavy chain variable region comprising an amino acid sequence having at least 80%, 85%, 90%, 95%, 96%, 96%, 97%, 98%, 99% identity to the amino acid sequence set forth in SEQ ID NO: 49; and a light chain variable region comprising an amino acid sequence having at least 80%, 85%, 90%, 95%, 96%, 97%, 98%, 99% identity to the amino acid sequence set forth in SEQ ID NO: 51.

In some embodiments, the antibody or antigen-binding fragment thereof comprises: an immunoglobulin heavy chain, comprising an amino acid sequence having at least 80%, 85%, 90%, 95%, 96%, 97%, 98%, 99% identity to the amino acid sequence set forth in SEQ ID NO: 55, and an immunoglobulin light chain, comprising an amino acid sequence having at least 80%, 85%, 90%, 95%, 96%, 97%, 98%, 99% identity to the amino acid sequence set forth in SEQ ID NO: 57.

In one aspect, disclosed herein is an antibody or antigen-binding fragment thereof that specifically binds to canine programmed death protein 1 (cPD-1), wherein the antibody or antigen-binding fragment thereof comprises: a heavy chain variable region comprising an amino acid sequence having at least 80%, 85%, 90%, 95%, 96%, 96%, 97%, 98%, 99% identity to the amino acid sequence set forth in SEQ ID NO: 49; and a light chain variable region comprising an amino acid sequence having at least 80%, 85%, 90%, 95%, 96%, 97%, 98%, 99% identity to the amino acid sequence set forth in SEQ ID NO: 51.

In one aspect, disclosed herein is an antibody or antigen-binding fragment thereof that specifically binds to canine programmed death protein 1 (cPD-1), wherein the antibody or antigen-binding fragment thereof comprises: a heavy chain variable region comprising the amino acid sequence set forth in SEQ ID NO: 49; and a light chain variable region comprising the amino acid sequence set forth in SEQ ID NO: 51.

In one aspect, disclosed herein is an antibody or antigen-binding fragment thereof that specifically binds to canine programmed death protein 1 (cPD-1), wherein the antibody or antigen-binding fragment thereof comprises: an immunoglobulin heavy chain, comprising an amino acid sequence having at least 80%, 85%, 90%, 95%, 96%, 97%, 98%, 99% identity to the amino acid sequence set forth in SEQ ID NO: 55, and an immunoglobulin light chain, comprising an amino acid sequence having at least 80%, 85%, 90%, 95%, 96%, 97%, 98%, 99% identity to the amino acid sequence set forth in SEQ ID NO: 57.

In one aspect, disclosed herein is an antibody or antigen-binding fragment thereof that specifically binds to canine programmed death protein 1 (cPD-1), wherein the antibody or antigen-binding fragment thereof comprises: an immunoglobulin heavy chain, comprising the amino acid sequence set forth in SEQ ID NO: 55, and an immunoglobulin light chain, comprising the amino acid sequence set forth in SEQ ID NO: 57.

In some embodiments, the antibody or antigen-binding fragment thereof is an IgG, an IgM, an IgE, an IgA, or an IgD molecule, or is derived from one of these. In some embodiments, the antibody or antigen-binding fragment thereof is selected from the group consisting of a full-length antibody, a Fab, a single-chain variable fragment (scFv), sc(Fv)2, dsFv, Fab, Fab′, (Fab′)2 and a diabody. In some embodiments, the antibody is a full-length antibody. In some embodiments, the antibody is a canine or caninized antibody. In some embodiments, the antibody is a canine antibody. In some embodiments, the antigen-binding fragment is an scFv. In some embodiments, the specific binding of the antibody or antigen-binding fragment thereof to cPD-1 disrupts the interaction of cPD-1 and a ligand of cPD-1 (e.g., canine programmed death-ligand 1).

In one aspect, disclosed herein is a canine antibody or antigen-binding fragment thereof that specifically binds to canine programmed death protein 1 (cPD-1).

In one aspect, disclosed herein is a canine antibody or antigen-binding fragment thereof that specifically binds to canine programmed death protein 1 (cPD-1), comprising the antibody or antigen-binding fragment thereof described herein. In some embodiments, the specific binding of the antibody or antigen-binding fragment thereof to cPD-1 disrupts the interaction of cPD-1 and a ligand of cPD-1 (e.g., canine programmed death-ligand 1).

In one aspect, disclosed herein is a single-chain variable fragment (scFv) specifically binds canine programmed death protein 1 (cPD-1), comprising: a heavy chain variable region that comprises heavy chain complementarity determining region 1 (HCDR1), HCDR2, and HCDR3, wherein HCDR1 comprises an amino acid sequence selected from the group consisting of SEQ ID NOs: 1, 27, and 43, HCDR2 comprises an amino acid sequence selected from the group consisting of SEQ ID NOs: 2, 28, and 44, and HCDR3 comprises an amino acid sequence selected from the group consisting of SEQ ID NOs: 3, 29, and 45; and a light chain variable region that comprises light chain complementarity determining region 1 (LCDR1), LCDR2, and LCDR3, wherein LCDR1 comprises an amino acid sequence selected from the group consisting of SEQ ID NOs: 4, 30, and 46, LCDR2 comprises an amino acid sequence selected from the group consisting of SEQ ID NOs: 5, 31 and 47, and LCDR3 comprises an amino acid sequence selected from the group consisting of SEQ ID NOs: 6, 32, and 48, wherein the heavy chain variable region and the light chain variable region are connected by a linker.

In one aspect, disclosed herein is a single-chain variable fragment (scFv) specifically binds to canine programmed death protein 1 (cPD-1), comprising: a heavy chain variable region comprising the amino acid sequence set forth in SEQ ID NOs: 7, 9, 33, or 49; and a light chain variable region comprising the amino acid sequence set forth in SEQ ID NOs: 11, 35, or 51, wherein the heavy chain variable region and the light chain variable region are connected by a linker.

In one aspect, disclosed herein is a single chain variable fragment (scFv) specifically binds to canine programmed death protein 1 (cPD-1), comprising the amino acid sequence set forth in SEQ ID NOs: 13, 15, 17, 37, or 53.

In some embodiments, the specific binding of the antibody or antigen-binding fragment thereof to cPD-1 disrupts the interaction of cPD-1 and a ligand of cPD-1 (e.g., canine programmed death-ligand 1).

In one aspect, disclosed herein is a bispecific molecule comprising the antibody or antigen-binding fragment thereof described herein, the canine antibody described herein, or the scFv described herein, linked to a molecule having a second binding specificity.

In one aspect, disclosed herein is an immunoconjugate comprising the antibody or antigen-binding fragment thereof described herein, the canine antibody described herein, or the scFv described herein, or the bispecific molecule described herein, linked to a therapeutic agent.

In one aspect, disclosed herein is an isolated nucleic acid encoding the antibody or antigen-binding fragment thereof described herein, the canine antibody described herein, or the scFv described herein.

In one aspect, disclosed herein is an isolated nucleic acid encoding an antibody or antigen-binding fragment thereof, wherein the antibody or antigen-binding fragment thereof comprises: a heavy chain variable region encoded by a nucleic acid comprising a polynucleotide sequence having at least 80%, 85%, 90%, 95%, 96%, 96%, 97%, 98%, 99% identity to the polynucleotide sequence set forth in SEQ ID NOs: 8, 10, 34, or 50; and a light chain variable region encoded by a nucleic acid comprising a polynucleotide sequence having at least 80%, 85%, 90%, 95%, 96%, 96%, 97%, 98%, 99% identity to the polynucleotide sequence set forth in SEQ ID NOs: 12, 36, or 52;

In some embodiments, the heavy chain variable region is encoded by a nucleic acid comprising the polynucleotide sequence set forth in SEQ ID NOs: 8, 10, 34, or 50. In some embodiments, the light chain variable region is encoded by a nucleic acid comprising the polynucleotide sequence set forth in SEQ ID NOs: 12, 36, or 52.

In one aspect, disclosed herein is an isolated nucleic acid encoding an antibody or antigen binding fragment thereof comprising: a heavy chain variable region encoded by a nucleic acid sequence comprising the polynucleotide sequence set forth in SEQ ID NO: 8; and a light chain variable region encoded by a nucleic acid sequence comprising the polynucleotide sequence set forth in SEQ ID NO: 12.

In one aspect, disclosed herein is an isolated nucleic acid encoding an antibody or antigen binding fragment thereof comprising: a heavy chain variable region encoded by a nucleic acid sequence comprising the polynucleotide sequence set forth in SEQ ID NO: 10; and a light chain variable region encoded by a nucleic acid sequence comprising the polynucleotide sequence set forth in SEQ ID NO: 12.

In one aspect, disclosed herein is an isolated nucleic acid encoding an antibody or antigen binding fragment thereof comprising: a heavy chain variable region encoded by a nucleic acid sequence comprising the polynucleotide sequence set forth in SEQ ID NO: 34; and a light chain variable region encoded by a nucleic acid sequence comprising the polynucleotide sequence set forth in SEQ ID NO: 36.

In one aspect, disclosed herein is an isolated nucleic acid encoding an antibody or antigen binding fragment thereof comprising: a heavy chain variable region encoded by a nucleic acid sequence comprising the polynucleotide sequence set forth in SEQ ID NO: 50; and a light chain variable region encoded by a nucleic acid sequence comprising the polynucleotide sequence set forth in SEQ ID NO: 52.

In some embodiments, the antibody or antigen-binding fragment thereof is an IgG, an IgM, an IgE, an IgA, or an IgD molecule, or is derived from one of these. In some embodiments, the antibody or antigen-binding fragment thereof is selected from the group consisting of a full-length antibody, a Fab, a single-chain variable fragment (scFv), sc(Fv)2, dsFv, Fab, Fab′, (Fab′)2 and a diabody. In some embodiments, the antibody is a full-length antibody. In some embodiments, the antibody is a canine or caninized antibody. In some embodiments, the antibody is a canine antibody.

In one aspect, disclosed herein is an isolated nucleic acid encoding a single-chain variable fragment (scFv) comprising: a heavy chain variable region comprising the nucleotide sequence set forth in SEQ ID NOs: 8, 10, 34, or 50; and a light chain variable region comprising the nucleotide sequence set forth in SEQ ID NOs: 12, 36, or 52, wherein the heavy chain variable region and the light chain variable region are connected by a linker.

In one aspect, disclosed herein is an isolated nucleic acid encoding a single-chain variable fragment (scFv) comprising the polynucleotide sequence set forth in SEQ ID NOs: 14, 16, 18, 38, or 54.

In some embodiments, the antibody or antigen-binding fragment thereof or the scFv specifically binds to canine programmed death protein 1 (cPD-1). In some embodiments, the specific binding of the antibody or antigen-binding fragment thereof or the scFv to cPD-1 disrupts the interaction of cPD-1 and a ligand of cPD-1 (e.g., canine programmed death-ligand 1).

In one aspect, disclosed herein is a vector comprising the isolated nucleic acid described herein. In some embodiments, the vector is an expression vector. In some embodiments, the vector is selected from the group consisting of a DNA vector, an RNA vector, a plasmid, a lentiviral vector, an adenoviral vector, an adeno-associated viral vector, and a retroviral vector.

In one aspect, disclosed herein is a host cell comprising the isolated nucleic acid described herein or the vector described herein. In some embodiments, the host cell is of eukaryotic or prokaryotic origin. In some embodiments, the host cell is of mammalian origin. In some embodiments, the host cell is of bacterial origin. In some embodiments, the host cell is a Chinese Hamster Ovary cell.

In one aspect, disclosed herein is a pharmaceutical composition comprising the antibody or antigen-binding fragment thereof described herein, the canine antibody described herein, or the scFv described herein, the bispecific molecule described herein, or the immunoconjugate described herein, and a pharmaceutically acceptable excipient, carrier, or diluent. In some embodiments, the pharmaceutical composition is formulated for intravenous administration. In some embodiments, the pharmaceutical composition is formulated for subcutaneous administration. In some embodiments, the pharmaceutical composition is formulated for intratumoral administration.

In one aspect, disclosed herein is a kit comprising the antibody or antigen-binding fragment thereof described herein, the canine antibody described herein, or the scFv described herein, the bispecific molecule described herein, the immunoconjugate described herein, or the pharmaceutical composition described herein, and an instruction for use.

In one aspect, disclosed herein is a method for increasing T cell proliferation and reducing T cell exhaustion in a subject in need thereof, comprising administering to the subject the antibody or antigen-binding fragment thereof described herein, the canine antibody described herein, or the scFv described herein, the bispecific molecule described herein, the immunoconjugate described herein, or the pharmaceutical composition described herein.

In one aspect, disclosed herein is a method for increasing IFNγ production in a subject in need thereof, comprising administering to the subject the antibody or antigen-binding fragment thereof described herein, the canine antibody described herein, or the scFv described herein, the bispecific molecule described herein, the immunoconjugate described herein, or the pharmaceutical composition described herein.

In one aspect, disclosed herein is a method for inhibiting canine programmed death protein 1 (cPD-1) signaling pathway in a subject in need thereof, comprising administering to the subject the antibody or antigen-binding fragment thereof described herein, the canine antibody described herein, or the scFv described herein, the bispecific molecule described herein, the immunoconjugate described herein, or the pharmaceutical composition described herein.

In some embodiments, the subject has cancer, sepsis or septic shock, or a chronic infection (e.g., viral infections).

In one aspect, disclosed herein is a method for treating a disease or condition in a subject in need thereof, comprising administering to the antibody or antigen-binding fragment thereof described herein, the canine antibody described herein, or the scFv described herein, the bispecific molecule described herein, the immunoconjugate described herein, or the pharmaceutical composition described herein. In some embodiments, the disease or condition comprises cancer, sepsis or septic shock, or a chronic infection (e.g., viral infections). In some embodiments, the method further comprising administering one or more additional therapeutics (e.g., chemotherapy) or interventions.

In some embodiments, the cancer is associated with canine programmed death protein 1 (cPD-1) signaling pathway. In some embodiments, a ligand for cPD-1 (e.g., canine programmed death-ligand 1) is expressed on a cancer cell of the subject. In some embodiments, cPD-1 is expressed on an immune cell of the subject. In some embodiments, the immune cell comprises a T cell (e.g., an activated T cell), a B cell, a Natural Killer (NK) cell, a regulatory T cell, a macrophage, or a Dendritic cell (DC). In some cases, the immune cell comprises an activated T cell. In some cases, the immune cell comprises a tumor-infiltrating lymphocyte. In some embodiments, the cancer comprises a melanoma (e.g., metastatic malignant melanoma), a prostate cancer (for example hormone refractory prostate adenocarcinoma), a head and neck cancer (for example, squamous cell carcinoma of the head and neck), a cervical cancer, a thyroid cancer, a glioblastoma, a glioma, leukemia, a lymphoma (for example, a B cell lymphoma), an adrenal gland cancer, an AIDS-associated cancer, an alveolar soft part sarcoma, an astrocytic tumor, bone cancer, a brain and spinal cord cancer, a metastatic brain tumor, a carotid body tumor, a chondrosarcoma, a chordoma, a chromophobe renal cell carcinoma, a clear cell carcinoma, cutaneous benign fibrous histiocytoma, a desmoplastic small round cell tumor, an ependymoma, a Ewing's tumor, an extra skeletal myxoid chondrosarcoma, a fibrogenesis imperfecta ossium, a fibrous dysplasia of the bone, a gallbladder or bile duct cancer, a gestational trophoblastic disease, a germ cell tumor, a hematological malignancy, hepatocellular carcinoma, an islet cell tumor, a Kaposi's sarcoma, a kidney cancer, a lipoma/benign lipomatous tumor, a liposarcoma/malignant lipomatous tumor, a medulloblastoma, a meningioma, a Merkel cell carcinoma, a multiple endocrine neoplasia, a multiple myeloma, a myelodysplasia syndrome, a neuroblastoma, a neuroendocrine tumor, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomyosarcoma, a sarcoma, a soft-tissue sarcoma, a squamous cell cancer, a stomach cancer, a synovial sarcoma, a testicular cancer, a thymic carcinoma, a thymoma, a thyroid metastatic cancer, a uterine cancer, or any combination thereof. In some embodiments, the cancer comprises cervical cancer, lung cancers, liver cancers, ovarian cancers, cancers of the skin including melanoma and squamous cell carcinoma, colon cancer, bladder cancer, breast cancer, kidney cancer, esophageal cancer, stomach cancer, pancreatic cancers, head cancer, and neck cancer.

In some embodiments, the antibody or antigen-binding fragment thereof, the canine antibody, the scFv, the bispecific molecule, or the immunoconjugate specifically binds to cPD-1. In some embodiments, the binding of antibody or antigen-binding fragment thereof, the canine antibody, the scFv, the bispecific molecule, or the immunoconjugate to cPD-1 disrupts the interaction of cPD-1 and a ligand of cPD-1 (e.g., canine programmed death-ligand 1). In some embodiments, the subject is canine.

In one aspect, disclosed herein is a method of making the pharmaceutical composition described herein, comprising admixing the antibody or antigen-binding fragment thereof described herein, the canine antibody described herein, or the scFv described herein, the bispecific molecule described herein, or the immunoconjugate described herein, and a pharmaceutically acceptable excipient, carrier, or diluent.

In one aspect, disclosed herein is a method of making a composition for treating cancer, sepsis or septic shock, or chronic infection (e.g., viral infections), comprising admixing the antibody or antigen-binding fragment thereof described herein, the canine antibody described herein, or the scFv described herein, the bispecific molecule described herein, or the immunoconjugate described herein, and a pharmaceutically acceptable excipient, carrier, or diluent. In some embodiments, the cancer comprises a melanoma (e.g., metastatic malignant melanoma), a prostate cancer (for example hormone refractory prostate adenocarcinoma), a head and neck cancer (for example, squamous cell carcinoma of the head and neck), a cervical cancer, a thyroid cancer, a glioblastoma, a glioma, leukemia, a lymphoma (for example, a B cell lymphoma), an adrenal gland cancer, an AIDS-associated cancer, an alveolar soft part sarcoma, an astrocytic tumor, bone cancer, a brain and spinal cord cancer, a metastatic brain tumor, a carotid body tumor, a chondrosarcoma, a chordoma, a chromophobe renal cell carcinoma, a clear cell carcinoma, cutaneous benign fibrous histiocytoma, a desmoplastic small round cell tumor, an ependymoma, a Ewing's tumor, an extra skeletal myxoid chondrosarcoma, a fibrogenesis imperfecta ossium, a fibrous dysplasia of the bone, a gallbladder or bile duct cancer, a gestational trophoblastic disease, a germ cell tumor, a hematological malignancy, hepatocellular carcinoma, an islet cell tumor, a Kaposi's sarcoma, a kidney cancer, a lipoma/benign lipomatous tumor, a liposarcoma/malignant lipomatous tumor, a medulloblastoma, a meningioma, a Merkel cell carcinoma, a multiple endocrine neoplasia, a multiple myeloma, a myelodysplasia syndrome, a neuroblastoma, a neuroendocrine tumor, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomyosarcoma, a sarcoma, a soft-tissue sarcoma, a squamous cell cancer, a stomach cancer, a synovial sarcoma, a testicular cancer, a thymic carcinoma, a thymoma, a thyroid metastatic cancer, a uterine cancer, or any combination thereof. In some embodiments, the cancer comprises cancer comprises cervical cancer, lung cancers, liver cancers, ovarian cancers, cancers of the skin including melanoma and squamous cell carcinoma, colon cancer, bladder cancer, breast cancer, kidney cancer, esophageal cancer, stomach cancer, pancreatic cancers, head cancer, and neck cancer.

In one aspect, disclosed herein is the antibody or antigen-binding fragment thereof described herein, the canine antibody described herein, or the scFv described herein, the bispecific molecule described herein, or the immunoconjugate described herein, or the pharmaceutical composition described herein, for use as a medicament.

In one aspect, disclosed herein is the antibody or antigen-binding fragment thereof described herein, the canine antibody described herein, or the scFv described herein, the bispecific molecule described herein, or the immunoconjugate described herein, or the pharmaceutical composition described herein, for use as a medicament for the treatment of cancer, sepsis or septic shock, or chronic infection (e.g., viral infections).

In one aspect, disclosed herein is use of the antibody or antigen-binding fragment thereof described herein, the canine antibody described herein, or the scFv described herein, the bispecific molecule described herein, or the immunoconjugate described herein, or the pharmaceutical composition described herein for the manufacture of a medicament.

In one aspect, disclosed herein is use of the antibody or antigen-binding fragment thereof described herein, the canine antibody described herein, or the scFv described herein, the bispecific molecule described herein, or the immunoconjugate described herein, or the pharmaceutical composition described herein for the manufacture of a medicament for the treatment of cancer, sepsis or septic shock, or chronic infection (e.g., viral infections).

In one aspect, disclosed herein is a method of producing the antibody or antigen-binding fragment thereof described herein, the canine antibody described herein, or the scFv described herein, the bispecific molecule described herein, comprising culturing the host cell described herein. In some embodiments, the method further comprises incubating the host cell described herein in a cell culture medium under conditions sufficient to allow expression and secretion of the antibody or antigen-binding fragment thereof described herein, the canine antibody described herein, or the scFv described herein.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The following detailed description of preferred embodiments of the present disclosure will be better understood when read in conjunction with the appended drawings. For the purpose of illustrating the present disclosure, there are shown in the drawings embodiments which are presently preferred. It should be understood, however, that the present disclosure is not limited to the precise arrangements and instrumentalities of the embodiments shown in the drawings.

FIG. 1 illustrates enrichment of a canine scFv phage display library after panning against canine PD-1 antigen. Recombinant canine PD-1 was adsorbed to a microtiter plate overnight at 4° C. Wells were washed and blocked with 2% milk in PBS (MPBS) for 1 hour at 37° C. The initial unpanned library (P0) and libraries of phage obtained after each round of selection (P1 through P4) were added to coated plates and incubated for 1 hour at 37° C. Plates were washed with PBS supplemented with 0.1% Tween (PBST) and bound phage was detected using a 1:5000 dilution of HRP-conjugated anti-M13 mAb in MPBS. Bound anti-M13 mAb was detected with ABTS substrate. OD was read at 405 nm after 30 min using a Molecular Devices SpectraMax 340 spectrophotometer.

FIGS. 2A-2B illustrate the binding of randomly selected scFv-expressing phage clones from panning rounds 3 and 4 to canine PD-1. Polyclonal phage from the fourth round of panning against cPD-1 were used as a positive control with wells coated with cPD-1 and negative control with wells coated with human (h) CD19 (FIG. 2A). Polyclonal phage from the third round of panning against hCD19 in an unrelated study were used as a positive control with wells coated with hCD19 and negative control against with wells coated with cPD-1 (FIG. 2A). In FIG. 2B, polyclonal phage from the fourth round of panning against canine (c) IL13Rα2 in an unrelated study along with wells coated with cPD-1 and cIL13Rα2 served similarly as negative and positive controls, respectively.

FIG. 3 illustrates the ability of unique soluble scFv clones to bind to cPD-1 by ELISA. 19 unique soluble scFv were tested for binding to cPD-1 by ELISA. Increasing amounts of biotinylated canine PD-1 were captured on streptavidin (SA)-coated ELISA plates. 0.25 μg/ml of soluble hemagglutinin (HA)-tagged scFv were added to the wells, and bound scFv was detected using an AP-conjugated anti-HA antibody. An irrelevant soluble HA tagged scFv against a MERS (Middle East Respiratory Syndrome) antigen from a different project was used as a negative control.

FIG. 4 illustrates a PD-1: PD-L1 inhibition assay. ELISA plates were coated with streptavidin. Soluble biotinylated canine PD-1 was incubated with increasing concentrations of canine PD-L1-Fc either alone or in the presence of selected soluble anti-cPD-1 scFv or an irrelevant soluble scFv against MERS. Pre-incubated mixtures were added to the ELISA plate and PD-L1 binding to PD-1 was detected using an anti-Fc-AP conjugate. Boxed and numbered clones inhibited PD-1: PD-L1 interactions.

FIG. 5 illustrates soluble canine anti-PD-1 scFv binding to cell surface-expressed PD-1. The scaffold human erythroleukemic cell line K562 was gene edited to eliminate the FcγRII (CD32) (KTδ32). Retroviral transduction of edited cells to express cPD-1 was performed and PD-1 positive target cells were selected in puromycin. Soluble, purified, HA-tagged scFvs were incubated with target cells and bound scFvs were detected using an anti-HA antibody. The soluble scFv against MERS and clone 3-7 that showed low affinity binding to cPD-1 by ELISA were used as negative controls.

FIG. 6 illustrates an evaluation of fully canine anti-PD-1 IgG4 binding to cell surface-expressed PD-1 by flow cytometry. Retroviral transduction of KTδ32 cells to express cPD-1 was performed and target positive cells were selected using puromycin. Fully canine IgG4-HA tagged clones against canine PD-1 or the irrelevant MERS antibody were used to stain KTδ32 and KTδ32.cPD-1. Bound IgG4 molecules were detected using an anti-HA antibody.

FIG. 7 illustrates that P3C6 antibody mutants bind to cPD-1. P3C6 was selected for further development and testing based on functional analysis and cell surface binding. To increase the yield/developability of P3C6, mutations were introduced into the framework regions. Full-length IgG were produced and the mutants were tested for their ability to bind to soluble cPD-1 by ELISA. P3C6mut3.1 and P3C6mut3.2 bear 4 and 5 amino acid substitutions, respectively, from the parent P3C6 clone with only one amino acid difference between the mutated sequences. P4B1 and P4-14 were used as positive controls.

FIG. 8 illustrates that P3C6 antibody mutants bind to cPD-1. Retroviral transduction of KTδ32 cells to express cPD-1 was performed and target positive cells were selected using puromycin. Cells were labeled with the two P3C6 mutants (mut3.1 and mut3.2), or P4B1 to confirm binding to cell surface expressed PD-1. Bound antibodies were detected using an anti-HA antibody. Irrelevant anti-MERS antibody was used as a negative control. Plots are gated on live, 7AAD-cells.

FIGS. 9A-9B show the yields of P3C6 antibody mutants from transient transfections of 293T cells.

FIG. 10 illustrates that P3C6 mutant antibodies augment canine T cell proliferative capacity. Canine PBMCs were labeled with cell trace violet (CTV) and stimulated with 2.5 μg/ml concanavalin A in the presence of P4B1, P3C6 (mut3.1 and mut3.2) full length IgG or the irrelevant MERS control antibody. Cells were labeled with anti-CD5 antibody and analyzed at 96 hr by flow cytometry. Plots are gated on live CD5⁺ cells. Data from one dog is shown. Cells show an increase in proliferative capacity and predicted fold expansion when stimulated in the presence of anti-PD-1 IgG compared to the MERS antibody.

FIG. 11 illustrates that P3C6 IgG4 mutants promote production of IFN-γ from mitogen activated canine T cells. Canine PBMCs were stimulated with 2.5 mg/ml concanavalin A in the presence of P4B1, P3C6 (mut3.1 and mut3.2) IgG4 or the irrelevant MERS antibody. Supernatants were harvested 96 hours later and assayed for the presence of IFN-γ by ELISA. Data from two dogs are shown. P3C6 mutants both show an increase in IFN-γ production when compared to the anti-MERS antibody and a second anti-PD-1 clone (P4B1).

FIG. 12 illustrates that clone A6 binds cell surface cPD-1. KTδ32 and KTδ32.cPD-1 cells were labeled with the two P3C6 mutants (mut3.1 and mut3.2), or Clone A6 reformatted as IgG4 to confirm binding to cell surface expressed cPD-1. Bound antibodies were detected using an anti-HA antibody. Antibody against the irrelevant MERS viral antigen was used as a negative control. Plots are gated on live, 7AAD-cells. Where indicated, clones were pre-incubated with soluble cPD-1 to block the antigen-binding sites. Pre-blocking resulted in abolishment of Clone A6 binding and a reduction of cell-surface labeling for clones P3C6 mut 3.1 and 3.2.

### DETAILED DESCRIPTION

Canine antibody phage display libraries can be used to identify antigen-specific single-chain variable fragment (scFv) fusion proteins that specifically bind to canine programmed death protein 1 (cPD-1). These canine PD-1-specific scFv then can be converted into full-length antibodies comprising canine-origin constant regions, making them suitable for in vivo use in canine subjects. The fully canine antibodies carry lower risk of inducing immune response in canine subjects than mouse antibodies, chimeric antibodies, or caninized antibodies. In some cases, the fully canine anti-PD-1 can be non-immunogenic and effective in checkpoint inhibition in treating canine cancer patients. Incorporation of such fully canine antibodies against canine PD-1 therapy into the veterinary arsenal can improve the outcome of pet dogs with malignancies.

Furthermore, the present disclosure can shed light on questions in human immune-oncology. Using fully canine anti-PD-1 in immune competent canine cancer patients with spontaneous tumors that share similar features to their human counterparts can yield informative results for human clinical trial design. The development of a fully canine anti-PD-1 provides an important comparative tool for translational research in human immune-oncology and enables certain combination therapies being evaluated to include checkpoint inhibition.

The present disclosure provides an antibody or antigen-binding fragment thereof that specifically binds to canine programmed death protein 1 (PD-1), comprising (a) a heavy chain complementarity determining region 1 (HCDR1), comprising the amino acid sequence set forth in SEQ ID NOs: 1, 27, or 43, (b) a heavy chain complementarity determining region 2 (HCDR2), comprising the amino acid sequence set forth in SEQ ID NOs: 2, 28, or 44, (c) a heavy chain complementarity determining region 3 (HCDR3), comprising the amino acid sequence set forth in SEQ ID NOs: 3, 29, or 45, (d) a light chain complementarity determining region 1 (LCDR1), comprising the amino acid sequence set forth in SEQ ID NOs: 4, 30, or 46, (e) a light chain complementarity determining region 2 (LCDR2), comprising the amino acid sequence set forth in SEQ ID NOs: 5, 31, or 47, and (f) a light chain complementarity determining region 3 (LCDR3), comprising the amino acid sequence set forth in SEQ ID NOs: 6, 32, or 48.

Disclosed herein, in some aspects, is an antibody or antigen-binding fragment thereof that specifically binds to canine programmed death protein 1 (PD-1), comprising: (a) a heavy chain variable region comprising an amino acid sequence having at least 80%, 85%, 90%, 95%, 96%, 96%, 97%, 98%, 99% identity to the amino acid sequence set forth in SEQ ID NOs: 7, 9, 33, or 49; and (b) a light chain variable region comprising an amino acid sequence having at least 80%, 85%, 90%, 95%, 96%, 97%, 98%, 99% identity to the amino acid sequence set forth in SEQ ID NOs: 11, 35, or 51.

Disclosed herein, in some aspects, is an antibody or antigen-binding fragment thereof that specifically binds to canine programmed death protein 1 (PD-1), comprising (a) an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, 23, 39, or 55, and (b) an immunoglobulin light chain, comprising an amino acid sequence having at least 80%, 85%, 90%, 95%, 96%, 97%, 98%, 99% identity to the amino acid sequence set forth in SEQ ID NOs: 25, 41, or 57.

In some aspects, provided herein is a canine antibody or antigen-binding fragments thereof that specifically binds to canine programmed death protein 1 (cPD-1).

In some aspects, provided herein is a single-chain variable fragment (scFv) specifically binds canine programmed death protein 1 (cPD-1) comprising (a) a heavy chain variable region that comprises heavy chain complementarity determining region 1 (HCDR1), HCDR2, and HCDR3, wherein HCDR1 comprises an amino acid sequence selected from the group consisting of SEQ ID NOs: 1, 27, and 43, HCDR2 comprises an amino acid sequence selected from the group consisting of SEQ ID NOs: 2, 28, and 44, and HCDR3 comprises an amino acid sequence selected from the group consisting of SEQ ID NOs: 3, 29, and 45; and (b) a light chain variable region that comprises light chain complementarity determining region 1 (LCDR1), LCDR2, and LCDR3, wherein LCDR1 comprises an amino acid sequence selected from the group consisting of SEQ ID NOs: 4, 30, and 46, LCDR2 comprises an amino acid sequence selected from the group consisting of SEQ ID NOs: 5, 31 and 47, and LCDR3 comprises an amino acid sequence selected from the group consisting of SEQ ID NOs: 6, 32, and 48, wherein the heavy chain variable region and the light chain variable region are connected by a linker.

In some aspects, provided herein is a single-chain variable fragment (scFv) comprising (a) a heavy chain variable region comprising the amino acid sequence set forth in SEQ ID NOs: 7, 9, 33, or 49; and (b) a light chain variable region comprising the amino acid sequence set forth in SEQ ID NOs: 11, 35, or 51, wherein the heavy chain variable region and the light chain variable region are connected by a linker.

In some aspects, provided herein is a single-chain variable fragment (scFv) specifically binds canine programmed death protein 1 (cPD-1) comprising the amino acid sequence set forth in SEQ ID NOs: 13, 15, 17, 37, or 53.

Also provided are bispecific molecules, immunoconjugates, nucleic acids, vectors, host cells, kits, compositions for the canine PD-1-specific antibodies and antigen-binding fragments or the scFv described herein, and methods of producing and using the same for the treatment of cancers in subjects (e.g., canine subjects).

### Definitions

In the detailed description, certain specific details are set forth in order to provide a thorough understanding of various embodiments. However, one skilled in the art will understand that the embodiments provided can be practiced without these details. Unless the context requires otherwise, throughout the specification and claims which follow, the word “comprise” and variations thereof, such as, “comprises” and “comprising” are to be construed in an open, inclusive sense, that is, as “including, but not limited to.” As used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise. Further, headings provided herein are for convenience only and do not interpret the scope or meaning of the claimed embodiments.

The articles “a” and “an” are used herein to refer to one or to more than one (i.e., to at least one) of the grammatical object of the article. By way of example, “an element” means one element or more than one element.

As used herein the term “about” refers to an amount that is near the stated amount by 10% or less. As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a dog or canine.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. Antibodies are typically tetramers of immunoglobulin molecules comprising two heavy chain and two light chain polypeptides. Each polypeptide chain contains three complementarity-determining regions (CDRs), which bind to the antigen and defines the antibody's antigen specificity.

As used herein, the term “antibody” and “antibodies” can also include polypeptides or polypeptide complexes derived from full-length antibodies. These polypeptide complexes can be naturally occurring or constructed from single chain antibodies or antibody fragments and retain an antigen-specific binding ability. The antibodies of the present disclosure can exist in a variety of forms including, for example, polyclonal antibodies, monoclonal antibodies, Fv, Fab and F(ab′)2, as well as single chain antibodies, scFv, caninized antibodies, canine antibodies, humanized antibodies, and human antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). For preparation of suitable antibodies, e.g., recombinant, monoclonal, or polyclonal antibodies, many techniques known in the art can be used (see, e.g., Kohler & Milstein, Nature 256:495-497 (1975); Kozbor et al., Immunology Today 4:72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies of this disclosure. Also, transgenic mice, or other organisms such as other mammals, can be used to express humanized or human antibodies as well as caninized or canine antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Herein a molecule, peptide, polypeptide, antibody, or antibody fragment can be referred to as “bispecific” or “dual-specific” including grammatical equivalents. A bispecific molecule possesses the ability to specifically bind to at least two structurally distinct targets. The specific binding can be the result of two distinct binding moieties that are structurally distinct at the molecular level, including but not limited to distinct non-identical amino acid sequences; or a single binding moiety that is able to specifically bind to two structurally distinct targets with high affinity (e.g., with a KD less than about 1×10⁻⁶). A molecule, peptide, polypeptide, antibody, or antibody fragment referred to as “multi-specific” refers to a molecule that possesses the ability to specifically bind to at least three structurally distinct targets. A “bispecific antibody” including grammatical equivalents refers to a bispecific molecule that preserves at least one fragment of an antibody able to specifically bind a target, for example, a variable region, heavy or light chain, or one or more complementarity determining regions from an antibody molecule. A “multi-specific antibody” including grammatical equivalents refers to a multi-specific molecule that preserves at least one fragment of an antibody able to specifically bind with a target, for example, a variable region, heavy or light chain, or complementarity determining region from an antibody molecule.

A “linker” herein is also referred to as “linker sequence” “spacer” “tethering sequence” or grammatical equivalents thereof. A “linker” as referred herein connects two distinct molecules that by themselves possess target binding, catalytic activity, or are naturally expressed and assembled as separate polypeptides, or comprise separate domains of the same polypeptide. For example, two distinct binding moieties or a heavy-chain/light-chain pair. A number of strategies can be used to covalently link molecules together. Linkers described herein can be utilized to join a light chain variable region and a heavy chain variable region in an scFv molecule; or can be used to tether an scFv or other antigen binding fragment on the N- or C-terminus of an antibody heavy chain; or the N- or C-terminus of a light chain to create a bispecific or multi-specific binding molecule. These include but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. The linker peptide can predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length or about 1 to 30 amino acids in length. In one embodiment, linkers of 1 to 20 amino acids in length can be used. Useful linkers include glycine-serine polymers, including for example (GS)n, (GSGGS)n (SEQ ID NO: 65), (GGGGS)n (SEQ ID NO: 67), and (GGGS)n (SEQ ID NO: 66), where n is an integer of at least one, glycine-alanine polymers, alanine-serine polymers, and other flexible linkers. Exemplary, linkers for linking antibody fragments or single chain variable fragments can include AAEPKSS (SEQ ID NO: 78), AAEPKSSDKTHTCPPCP (SEQ ID NO: 79), GGGG (SEQ ID NO: 80), or GGGGDKTHTCPPCP (SEQ ID NO: 81). Alternatively, a variety of non-proteinaceous polymers, including but not limited to polyethylene glycol (PEG), polypropylene glycol, polyoxyalkylenes, or copolymers of polyethylene glycol and polypropylene glycol, can find use as linkers.

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDR-H1, CDR-H2, CDR-H3) and three CDRs in each light chain variable region (CDR-L1, CDR-L2, CDR-L3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 Jan.; 27 (1): 55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309 (3): 657-70, (“Aho” numbering scheme); and Whitelegg N R and Rees A R, “WAM: an improved algorithm for modelling antibodies on the WEB,” Protein Eng. 2000 Dec.; 13 (12): 819-24 (“AbM” numbering scheme. In certain embodiments, the CDRs of the antibodies described herein can be defined by a method selected from Kabat, Chothia, IMGT, Aho, AbM, or combinations thereof.

The boundaries of a given CDR or FR can vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. In certain embodiments, the CDRs of the antibodies described herein can be defined by IMGT method.

The term “variable region” or “variable domain” refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs (See e.g., Kindt et al. Kuby Immunology, 6th ed., W. H. Freeman and Co., page 91 (2007)). A single V_{H} or V_{L} domain can be sufficient to confer antigen-binding specificity. Furthermore, antibodies that bind a particular antigen can be isolated using a V_{H} or V_{L} domain from an antibody that binds the antigen to screen a library of complementary V_{L} or V_{H} domains, respectively (See e.g., Portolano et al., J. Immunol. 150:880-887 (1993); Clarkson et al., Nature 352:624-628 (1991)).

Specific binding or binding of antibody molecules described herein refers to binding mediated by one or more CDR portions of the antibody. Not all CDRs may be required for specific binding. Specific binding can be demonstrated for example by an ELISA against a specific recited target or antigen that shows significant increase in binding compared to an isotype control antibody.

An “epitope” refers to the binding determinant of an antibody or fragment described herein minimally necessary for specific binding of the antibody or fragment thereof to a target antigen. When the target antigen is a polypeptide, the epitope will be a continuous or discontinuous epitope. A continuous epitope is formed by one region of the target antigen, while a discontinuous epitope can be formed from two or more separate regions. A discontinuous epitope, for example, can form when a target antigen adopts a tertiary structure that brings two amino acid sequences together and forms a three-dimensional structure bound by the antibody. When the target antigen is a polypeptide, the epitope will generally be a plurality of amino acids linked into a polypeptide chain. A continuous epitope can comprise 5, 6, 7, 8, 9, 10, 11, 12, 13, 14, 15, 16, 17, 18, 19, or 20 contiguous amino acids. While an epitope can comprise a contiguous polymer of amino acids, not every amino acid of the polymer can be contacted by an amino acid residue of the antibody. Such non-contacted amino acids will still comprise part of the epitope as they can be important for the structure and linkage of the contacted amino acids. The skilled artisan can determine if any given antibody binds an epitope of a reference antibody, for example, by cross-blocking experiments with a reference antibody. In certain embodiments, described herein, are antibodies that bind the same epitope of the described antibodies. In certain embodiments, described herein, are antibodies that are competitively blocked by the described antibodies. In certain embodiments, described herein, are antibodies that compete for binding with the described antibodies.

The term “antibody fragment” refers to a polypeptide comprising or derived from a portion of an intact antibody and comprises the antigen-binding determining variable regions of an intact antibody. Examples of antibody fragments include, but are not limited to, Fab, Fab′, F(ab′)2, and Fv fragments, linear antibodies, scFv antibodies, single-domain antibodies, such as camelid antibodies (Riechmann, 1999, Journal of Immunological Methods 231:25-38), composed of either a V_{L} or a V_{H} domain which exhibit sufficient affinity for the target, and multi-specific antibodies formed from antibody fragments. Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., polypeptide linkers, and/or those that are not produced by enzyme digestion of a naturally-occurring intact antibody. In some aspects, the antibody fragments are scFvs.

A Fab or Fab fragment contains the constant domain of the light chain and the first constant domain (CH1) of the heavy chain. Fab′ or Fab′ fragments differ from Fab fragments by the addition of a few residues at the carboxyl terminus of the heavy chain CH1 domain including one or more cysteines from the antibody hinge region. Fab′ fragments are produced by cleavage of the disulfide bond at the hinge cysteines of the F(ab′)2 pepsin digestion product. Additional chemical couplings of antibody fragments are known to those of ordinary skill in the art. Fab and F(ab′)2 fragments lack the Fragment crystallizable (Fc) region of an intact antibody, clear more rapidly from the circulation of animals, and can have less nonspecific tissue binding than an intact antibody. “Fv” fragment is the minimum fragment of an antibody that contains a complete target recognition and binding site. This region consists of a dimer of one heavy and one light chain variable domain in a tight, non-covalent association (V_{H}-V_{L} dimer). It is in this configuration that the three CDRs of each variable domain interact to define a target binding site on the surface of the VHVL dimer. In some cases, the six CDRs confer target binding specificity to the antibody. However, in some cases. even a single variable domain (or half of an Fv comprising only three CDRs specific for a target) can have the ability to recognize and bind target. Single domain antibodies (sdAb)/single-chain fragments are composed of a single V_{H} or V_{L} domain which exhibit sufficient affinity to an antigen. The antibody fragment also includes a canine antibody or a caninized antibody or a portion of a canine antibody or a caninized antibody. A scFv (Single-chain Fv) refers to antibody binding fragments comprise the V_{H} and V_{L} domains of an antibody, where these domains are present in a single polypeptide chain. Generally, the scFv polypeptide further comprises a polypeptide linker between the V_{H} and V_{L} domains which enables the scFv to form a structure favorable for target binding.

The term “diabodies” refers to small antibody fragments prepared by constructing scFv fragments with short linkers (about 5-10 residues) between the V_{H} and V_{L} domains such that inter-chain but not intra-chain pairing of the variable domains is achieved, resulting in a bivalent fragment, i.e., fragment having two antigen-binding sites. Bispecific diabodies are heterodimers of two “crossover” scFv fragments in which the V_{H} and V_{L} domains of the two antibodies are present on different polypeptide chains.

The term “linear antibodies” generally refers to the antibodies comprise a pair of tandem Fd segments (VH-CH1-VH-CH1) which, together with complementary light chain polypeptides, form a pair of antigen binding regions. Linear antibodies can be bispecific or monospecific.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations. κ and λ light chains refer to the two major antibody light chain isotypes.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” or “Ag” as used herein is defined as a molecule that provokes an immune response. This immune response can involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect which can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies of the present disclosure in prevention of the occurrence of tumor in the first place.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In certain embodiments, the cancer is medullary thyroid carcinoma.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody of the present disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the present disclosure can be replaced with other amino acid residues from the same side chain family and the altered antibody can be tested for the ability to bind GFRα4 using the functional assays described herein.

“Co-stimulatory ligand”, as the term is used herein, includes a molecule expressed by an antigen presenting cell (e.g., an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40L, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to cell-surface molecules expressed by T cells that specifically bind with co-stimulatory ligands expressed by antigen-presenting cells (APCs), thereby providing a “secondary signal” which, in combination with the “primary signal” delivered through MHC/HLA-antigen interactions with the T Cell Receptor (TCR) results in optimal T cell activation including, but not limited to, cytokine production and proliferation. Co-stimulatory molecules include, but are not limited to CD27, CD28, 4-1BB, OX40, CD30, CD40L, PD-1, ICOS, lymphocyte function-associated antigen-1 (LFA-1), CD2, CD7, LIGHT, NKG2C, B7-H3, and a ligand that specifically binds with CD83

The term “dysregulated” when used in the context of the level of expression or activity of PD-1 or it's ligands PD-L1 and PD-L2 refers to the level of expression or activity that is different from the expression level or activity of PD-1 or it's ligands in an otherwise identical healthy animal, organism, tissue, cell or component thereof. The term “dysregulated” also refers to the altered regulation of the level of expression and activity of PD-1, PD-L1, compared to the regulation in an otherwise identical healthy animal, organism, tissue, cell or component thereof.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA can include introns.

“Effective amount” or “therapeutically effective amount” are used interchangeably herein, and refer to an amount of a compound, formulation, material, or composition, as described herein effective to achieve a particular biological result. Such results can include, but are not limited to, the inhibition of virus infection as determined by any means suitable in the art.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” as used herein, refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” or “caninized” and “chimeric” forms of non-human or non-canine (e.g., mouse) antibodies are immunoglobulins, immunoglobulin chains or fragments thereof (such as Fv, Fab, Fab′, F(ab′)2 or other antigen-binding subsequences of antibodies) which contain minimal sequences derived from non-human or non-canine immunoglobulin. For the most part, humanized, caninized, and chimeric antibodies are human or canine immunoglobulins (recipient antibody) in which residues from a complementary-determining region (CDR) of the recipient are replaced by residues from a CDR of a non-human or non-canine species (donor antibody) such as mouse, rat or rabbit having the desired specificity, affinity, and capacity. In some instances, Fv framework region (FR) residues of the human or canine immunoglobulin are replaced by corresponding non-human or non-canine residues. Furthermore, humanized, caninized, and chimeric antibodies can comprise residues which are found neither in the recipient antibody nor in the imported CDR or framework sequences. These modifications are made to further refine and optimize antibody performance. In general, the humanized, caninized, and chimeric antibody will comprise substantially all of at least one, and typically two, variable domains, in which all or substantially all of the CDR regions correspond to those of a non-human immunoglobulin and all or substantially all of the FR regions are those of a human or canine immunoglobulin sequence. The caninized and chimeric antibody optimally also will comprise at least a portion of an immunoglobulin constant region (Fc), typically that of a canine immunoglobulin.

“Fully canine” or “canine antibody” refers to an immunoglobulin, such as an antibody, with an amino acid sequence corresponding to that of an antibody produced by a canine or a canine cell, or non-canine source that utilizes canine antibody repertoires or other canine antibody-encoding sequences, including canine antibody libraries. The term excludes caninized forms of non-canine antibodies comprising non-canine antigen-binding regions, such as those in which all or substantially all CDRs are non-canine. In some cases, a fully canine antibody does not include a portion of an antibody sequence from non-canine species. Canine antibodies can be prepared by administering an immunogen to a transgenic animal that has been modified to produce intact canine antibodies or intact antibodies with canine variable regions in response to antigenic challenge. Such animals typically contain all or a portion of the canine immunoglobulin loci, which replace the endogenous immunoglobulin loci, or which are present extrachromosomally or integrated randomly into the animal's chromosomes. In such transgenic animals, the endogenous immunoglobulin loci have generally been inactivated. Canine antibodies also can be derived from canine antibody libraries, including phage display and cell-free libraries, containing antibody-encoding sequences derived from a canine repertoire.

As used herein, an “instructional material” includes a publication, a recording, a diagram, or any other medium of expression which can be used to communicate the usefulness of the compositions and methods of the present disclosure. The instructional material of the kit of the present disclosure can, for example, be affixed to a container which contains the nucleic acid, peptide, and/or composition of the present disclosure or be shipped together with a container which contains the nucleic acid, peptide, and/or composition. Alternatively, the instructional material can be shipped separately from the container with the intention that the instructional material and the compound be used cooperatively by the recipient.

“Identity” as used herein refers to the percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or can be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA can also include introns to the extent that the nucleotide sequence encoding the protein can in some version contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence can be the core promoter sequence and in other instances, this sequence can also include an enhancer sequence and other regulatory elements which are required for expression of the gene product. The promoter/regulatory sequence can, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide encodes or specified by a gene, causes the gene product to be produced in a cell substantially only if the cell is a cell of the tissue type corresponding to the promoter.

A “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the plasma membrane of a cell. An example of a “cell surface receptor” is human GFRα4.

“Single chain antibodies” refer to antibodies formed by recombinant DNA techniques in which immunoglobulin heavy and light chain fragments are linked to each other using an engineered span of amino acids to recapitulate the Fv region of an antibody as a single polypeptide. Various methods of generating single chain antibodies are known, including those described in U.S. Pat. No. 4,694,778; Bird (1988) Science 242:423-442; Huston et al. (1988) Proc. Natl. Acad. Sci. USA 85:5879-5883; Ward et al. (1989) Nature 334:54454; Skerra et al. (1988) Science 242:1038-1041.

The term “subject” is intended to include living organisms in which an immune response can be elicited (e.g., mammals). A “subject” or “patient,” as used therein, can be a human or non-human mammal. Non-human mammals include, for example, livestock and pets, such as ovine, bovine, porcine, canine, feline and murine mammals. Preferably, the subject is canine.

As used herein, a “substantially purified” cell is a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In other embodiments, the cells are not cultured in vitro.

The term “therapeutic” as used herein means a treatment and/or prophylaxis. A therapeutic effect is obtained by suppression, remission, or eradication of a disease state.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, lentiviral vectors, and the like.

By the term “specifically binds,” as used herein, is meant an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-β, and/or reorganization of cytoskeletal structures, and the like.

A “stimulatory molecule,” as the term is used herein, means a molecule on a T cell that specifically binds with a cognate stimulatory ligand present on an antigen presenting cell and/or on a tumor cell.

A “stimulatory ligand,” as used herein, means a ligand that when present on an antigen presenting cell (e.g., an aAPC, a dendritic cell, a B-cell, and the like) or a tumor cell, can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a super-agonist anti-CD28 antibody, and a super-agonist anti-CD2 antibody.

Ranges: throughout this present disclosure, various aspects of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Binding Polypeptides, Antibodies, and scFvs

In aspects, disclosed herein are antibodies or antigen-binding fragment thereof (or “binding polypeptides,” which is used interchangeably) which are characterized by particular functional features or properties of the antibodies or antigen-binding fragment thereof. For example, the binding polypeptides and antibodies specifically bind to canine programmed death protein 1 (“cPD-1” or “canine PD-1” are used interchangeably). The binding polypeptides and antibodies of the present disclosure can bind to canine PD-1 with high affinity. The binding polypeptides and antibodies of the present disclosure can specifically recognize naturally expressed canine PD-1 protein on a cell. In some cases, the binding polypeptides and antibodies of the present disclosure do not cross-react to other surface molecules on such cell. The binding polypeptides and antibodies of the present disclosure can specifically bind to naturally expressed canine PD-1 protein on a cell, so that such binding prevents canine PD-1 from binding to a natural ligand of canine PD-1, such as canine programmed death-ligand 1 (“cPD-L1” or “canine PD-L1). In some cases, the cell is a tumor cell (e.g., melanoma, non-small cell lung carcinoma and hepatocellular carcinoma). In some cases, the cell is an immune cell. In some cases, the immune cell comprises a T cell (e.g., an activated T cell), a B cell, a Natural Killer (NK) cell, a regulatory T cell, a macrophage, or a Dendritic cell (DC). In some cases, the immune cell comprises an activated T cell. In some cases, the immune cell comprises a tumor-infiltrating lymphocyte. In some cases, the cPD-L1 is overexpressed on a tumor cell. In some cases, the antibody or the antigen-binding fragment thereof can bind to cPD-1 and inhibit cPD-1 signaling pathway.

In certain aspects, the present disclosure provides an antibody or antigen-binding fragment thereof that specifically binds to canine programmed death protein 1 (PD-1). In some cases, the antibody or antigen-binding fragment thereof comprises a heavy chain complementarity determining region 1 (HCDR1). In some cases, the antibody or antigen-binding fragment thereof comprises a heavy chain complementarity determining region 2 (HCDR2). In some cases, the antibody or antigen-binding fragment thereof comprises a heavy chain complementarity determining region 3 (HCDR3). In some cases, the antibody or antigen-binding fragment thereof comprises a light chain complementarity determining region 1 (LCDR1). In some cases, the antibody or antigen-binding fragment thereof comprises a light chain complementarity determining region 2 (LCDR2). In some cases, the antibody or antigen-binding fragment thereof comprises a light chain complementarity determining region 3 (LCDR3). In some cases, the antibody or antigen-binding fragment thereof comprises an antigen-binding domain that specifically binds to canine programmed death protein 1 (PD-1). In certain embodiments, the antigen-binding domain comprises a heavy chain variable region that comprises three heavy chain complementarity determining regions (HCDRs) and a light chain variable region that comprises three light chain complementarity determining regions (LCDRs).

In certain aspects, the present disclosure provides an isolated binding polypeptide comprising an HCDR1 comprising the amino acid sequences set forth in SEQ ID NOs: 1, 27, or 43. Also provided is an isolated binding polypeptide comprising an HCDR2 comprising the amino acid sequences set forth in SEQ ID NOs: 2, 28, or 44. Also provided is an isolated binding polypeptide comprising an HCDR3 comprising the amino acid sequences set forth in SEQ ID NOs: 3, 29, or 45. Also provided is an isolated binding polypeptide comprising a light chain variable region that comprises an LCDR1 comprising the amino acid sequences set forth in SEQ ID NOs: 4, 30, or 46. Also provided is an isolated binding polypeptide comprising an LCDR2 comprising the amino acid sequences set forth in SEQ ID NOs: 5, 31, or 47. Also provided is an isolated binding polypeptide comprising an LCDR3 comprising the amino acid sequences set forth in SEQ ID NOs: 6, 32, or 48.

In certain aspects, the present disclosure provides an antibody or antigen-binding fragment thereof comprising a heavy chain complementarity determining region 1 (HCDR1), comprising the amino acid sequence set forth in SEQ ID NOs: 1, 27, or 43, a heavy chain complementarity determining region 2 (HCDR2), comprising the amino acid sequence set forth in SEQ ID NOs: 2, 28, or 44, a heavy chain complementarity determining region 3 (HCDR3), comprising the amino acid sequence set forth in SEQ ID NOs: 3, 29, or 45, a light chain complementarity determining region 1 (LCDR1), comprising the amino acid sequence set forth in SEQ ID NOs: 4, 30, or 46, a light chain complementarity determining region 2 (LCDR2), comprising the amino acid sequence set forth in SEQ ID NOs: 5, 31, or 47, and a light chain complementarity determining region 3 (LCDR3), comprising the amino acid sequence set forth in SEQ ID NOs: 6, 32, or 48. In some cases, the antibody or antigen-binding fragment thereof specifically binds to canine programmed death protein 1 (PD-1). In some cases, the antibody or antigen-binding fragment comprises a heavy chain variable region comprising an amino acid sequence having at least 80%, 85%, 90%, 95%, 96%, 96%, 97%, 98%, 99% identity to the amino acid sequence set forth in SEQ ID NOs: 7, 9, 33, or 49. In some cases, the antibody or antigen-binding fragment comprises a light chain variable region comprising an amino acid sequence having at least 80%, 85%, 90%, 95%, 96%, 97%, 98%, 99% identity to the amino acid sequence set forth in SEQ ID NOs: 11, 35, or 51. In some cases, the antibody or antigen-binding fragment comprises an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, 23, 39, or 55. In some cases, the antibody or antigen-binding fragment comprises an immunoglobulin light chain, comprising an amino acid sequence having at least 80%, 85%, 90%, 95%, 96%, 97%, 98%, 99% identity to the amino acid sequence set forth in SEQ ID NOs: 25, 41, or 57.

In certain aspects, the present disclosure provides an antibody or antigen-binding fragment thereof comprising a heavy chain variable region comprising an amino acid sequence having at least 80%, 85%, 90%, 95%, 96%, 96%, 97%, 98%, 99% identity to the amino acid sequence set forth in SEQ ID NOs: 7, 9, 33, or 49 and a light chain variable region comprising an amino acid sequence having at least 80%, 85%, 90%, 95%, 96%, 97%, 98%, 99% identity to the amino acid sequence set forth in SEQ ID NOs: 11, 35, or 51. In some cases, the antibody or antigen-binding fragment thereof specifically binds to canine programmed death protein 1 (PD-1).

In certain aspects, the present disclosure provides an antibody or antigen-binding fragment thereof comprising a heavy chain variable region comprising the amino acid sequence set forth in SEQ ID NOs: 7, 9, 33, or 49; and a light chain variable region comprising the amino acid sequence set forth in SEQ ID NOs: 11, 35, or 51. In some cases, the antibody or antigen-binding fragment thereof specifically binds to canine programmed death protein 1 (PD-1).

In certain aspects, the present disclosure provides an antibody or antigen-binding fragment thereof comprising an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, 23, 39, or 55, and an immunoglobulin light chain, comprising an amino acid sequence having at least 80%, 85%, 90%, 95%, 96%, 97%, 98%, 99% identity to the amino acid sequence set forth in SEQ ID NOs: 25, 41, or 57. In some cases, the antibody or antigen-binding fragment thereof specifically binds to canine programmed death protein 1 (PD-1).

In certain aspects, the present disclosure provides an antibody or antigen-binding fragment thereof comprising an immunoglobulin heavy chain, comprising the amino acid sequence set forth in SEQ ID NOs: 19, 21, 23, 39, or 55, and an immunoglobulin light chain, comprising the amino acid sequence set forth in SEQ ID NOs: 25, 41, or 57. In some cases, the antibody or antigen-binding fragment thereof specifically binds to canine programmed death protein 1 (PD-1).

In certain aspects, the present disclosure provides an isolated binding polypeptide comprising an HCDR1 comprising the amino acid sequence set forth in SEQ ID NO: 1, an HCDR2 comprising the amino acid sequence set forth in SEQ ID NO: 2, an HCDR3 comprising the amino acid sequence set forth in SEQ ID NO: 3, an LCDR1 comprising the amino acid sequence set forth in SEQ ID NO: 4, an LCDR2 comprising the amino acid sequence set forth in SEQ ID NO: 5, and an LCDR3 comprising the amino acid sequence set forth in SEQ ID NO: 6.

In some embodiments, the isolated binding polypeptide is an antibody or antigen-binding fragment thereof that specifically binds to canine programmed death protein 1 (PD-1). In some embodiments, the antibody or antigen-binding fragment thereof comprises a heavy chain variable region comprising an amino acid sequence having at least 80%, 85%, 90%, 95%, 96%, 96%, 97%, 98%, 99% identity to the amino acid sequence set forth in SEQ ID NOs: 7 or 9. In some embodiments, the antibody or antigen-binding fragment thereof comprises a light chain variable region comprising an amino acid sequence having at least 80%, 85%, 90%, 95%, 96%, 97%, 98%, 99% identity to the amino acid sequence set forth in SEQ ID NO: 11. In some embodiments, the antibody or antigen-binding fragment thereof comprises an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, or 23. In some embodiments, the antibody or antigen-binding fragment thereof comprises an immunoglobulin light chain, comprising an amino acid sequence having at least 80%, 85%, 90%, 95%, 96%, 97%, 98%, 99% identity to the amino acid sequence set forth in SEQ ID NO: 25. In certain aspects, the present disclosure provides an antibody or antigen-binding fragment thereof comprises a heavy chain variable region comprising an amino acid sequence having at least 80%, 85%, 90%, 95%, 96%, 96%, 97%, 98%, 99% identity to the amino acid sequence set forth in SEQ ID NOs: 7 or 9; and a light chain variable region comprising an amino acid sequence having at least 80%, 85%, 90%, 95%, 96%, 97%, 98%, 99% identity to the amino acid sequence set forth in SEQ ID NO: 11. In some cases, the antibody or antigen-binding fragment thereof specifically binds to canine programmed death protein 1 (PD-1).

In certain aspects, the present disclosure provides an antibody or antigen-binding fragment thereof comprises a heavy chain variable region comprising the amino acid sequence set forth in SEQ ID NO: 7; and a light chain variable region comprising the amino acid sequence set forth in SEQ ID NO: 11. In some cases, the antibody or antigen-binding fragment thereof specifically binds to canine programmed death protein 1 (PD-1).

In certain aspects, the present disclosure provides an antibody or antigen-binding fragment thereof comprises a heavy chain variable region comprising the amino acid sequence set forth in SEQ ID NO: 9; and a light chain variable region comprising the amino acid sequence set forth in SEQ ID NO: 11. In some cases, the antibody or antigen-binding fragment thereof specifically binds to canine programmed death protein 1 (PD-1).

In certain aspects, the present disclosure provides an antibody or antigen-binding fragment thereof comprises an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, or 23, and an immunoglobulin light chain, comprising an amino acid sequence having at least 80%, 85%, 90%, 95%, 96%, 97%, 98%, 99% identity to the amino acid sequence set forth in SEQ ID NO: 25. In some cases, the antibody or antigen-binding fragment thereof specifically binds to canine programmed death protein 1 (PD-1).

In certain aspects, the present disclosure provides an antibody or antigen-binding fragment thereof comprises an immunoglobulin heavy chain, comprising the amino acid sequence set forth in SEQ ID NOs: 19, and an immunoglobulin light chain, comprising the amino acid sequence set forth in SEQ ID NO: 25. In some cases, the antibody or antigen-binding fragment thereof specifically binds to canine programmed death protein 1 (PD-1).

In certain aspects, the present disclosure provides an antibody or antigen-binding fragment thereof comprises an immunoglobulin heavy chain, comprising the amino acid sequence set forth in SEQ ID NOs: 21, and an immunoglobulin light chain, comprising the amino acid sequence set forth in SEQ ID NO: 25. In some cases, the antibody or antigen-binding fragment thereof specifically binds to canine programmed death protein 1 (PD-1).

In certain aspects, the present disclosure provides an antibody or antigen-binding fragment thereof comprises an immunoglobulin heavy chain, comprising the amino acid sequence set forth in SEQ ID NOs: 23, and an immunoglobulin light chain, comprising the amino acid sequence set forth in SEQ ID NO: 25. In some cases, the antibody or antigen-binding fragment thereof specifically binds to canine programmed death protein 1 (PD-1).

In certain aspects, the present disclosure provides an isolated binding polypeptide comprising an HCDR1 comprising the amino acid sequence set forth in SEQ ID NO: 27, an HCDR2 comprising the amino acid sequence set forth in SEQ ID NO: 28, an HCDR3 comprising the amino acid sequence set forth in SEQ ID NO: 29, an LCDR1 comprising the amino acid sequence set forth in SEQ ID NO: 30, an LCDR2 comprising the amino acid sequence set forth in SEQ ID NO: 31, and an LCDR3 comprising the amino acid sequence set forth in SEQ ID NO: 32. In some embodiments, the isolated binding polypeptide is an antibody or antigen-binding fragment thereof that specifically binds to canine programmed death protein 1 (PD-1). In some embodiments, the antibody or antigen-binding fragment thereof comprises a heavy chain variable region comprising an amino acid sequence having at least 80%, 85%, 90%, 95%, 96%, 96%, 97%, 98%, 99% identity to the amino acid sequence set forth in SEQ ID NO: 33. In some embodiments, the antibody or antigen-binding fragment thereof comprises a light chain variable region comprising an amino acid sequence having at least 80%, 85%, 90%, 95%, 96%, 97%, 98%, 99% identity to the amino acid sequence set forth in SEQ ID NO: 35. In some embodiments, the antibody or antigen-binding fragment thereof comprises an immunoglobulin heavy chain, comprising an amino acid sequence having at least 80%, 85%, 90%, 95%, 96%, 97%, 98%, 99% identity to the amino acid sequence set forth in SEQ ID NO: 39. In some embodiments, the antibody or antigen-binding fragment thereof comprises an immunoglobulin light chain, comprising an amino acid sequence having at least 80%, 85%, 90%, 95%, 96%, 97%, 98%, 99% identity to the amino acid sequence set forth in SEQ ID NO: 41.

In certain aspects, the present disclosure provides an antibody or antigen-binding fragment thereof comprises a heavy chain variable region comprising an amino acid sequence having at least 80%, 85%, 90%, 95%, 96%, 96%, 97%, 98%, 99% identity to the amino acid sequence set forth in SEQ ID NO: 33; and a light chain variable region comprising an amino acid sequence having at least 80%, 85%, 90%, 95%, 96%, 97%, 98%, 99% identity to the amino acid sequence set forth in SEQ ID NO: 35. In some cases, the antibody or antigen-binding fragment thereof specifically binds to canine programmed death protein 1 (PD-1).

In certain aspects, the present disclosure provides an antibody or antigen-binding fragment thereof comprises a heavy chain variable region comprising the amino acid sequence set forth in SEQ ID NO: 33; and a light chain variable region comprising the amino acid sequence set forth in SEQ ID NO: 35. In some cases, the antibody or antigen-binding fragment thereof specifically binds to canine programmed death protein 1 (PD-1).

In certain aspects, the present disclosure provides an antibody or antigen-binding fragment thereof comprises an immunoglobulin heavy chain, comprising an amino acid sequence having at least 80%, 85%, 90%, 95%, 96%, 97%, 98%, 99% identity to the amino acid sequence set forth in SEQ ID NO: 39, and an immunoglobulin light chain, comprising an amino acid sequence having at least 80%, 85%, 90%, 95%, 96%, 97%, 98%, 99% identity to the amino acid sequence set forth in SEQ ID NO: 41. In some cases, the antibody or antigen-binding fragment thereof specifically binds to canine programmed death protein 1 (PD-1).

In certain aspects, the present disclosure provides an antibody or antigen-binding fragment thereof comprises an immunoglobulin heavy chain, comprising the amino acid sequence set forth in SEQ ID NO: 39, and an immunoglobulin light chain, comprising the amino acid sequence set forth in SEQ ID NO: 41. In some cases, the antibody or antigen-binding fragment thereof specifically binds to canine programmed death protein 1 (PD-1).

In certain aspects, the present disclosure provides an isolated binding polypeptide comprising an HCDR1 comprising the amino acid sequence set forth in SEQ ID NO: 43, an HCDR2 comprising the amino acid sequence set forth in SEQ ID NO: 44, an HCDR3 comprising the amino acid sequence set forth in SEQ ID NO: 45, an LCDR1 comprising the amino acid sequence set forth in SEQ ID NO: 46, an LCDR2 comprising the amino acid sequence set forth in SEQ ID NO: 47, and an LCDR3 comprising the amino acid sequence set forth in SEQ ID NO: 48. In some embodiments, the isolated binding polypeptide is an antibody or antigen-binding fragment thereof that specifically binds to canine programmed death protein 1 (PD-1). In some embodiments, the antibody or antigen-binding fragment thereof comprises a heavy chain variable region comprising an amino acid sequence having at least 80%, 85%, 90%, 95%, 96%, 96%, 97%, 98%, 99% identity to the amino acid sequence set forth in SEQ ID NO: 49. In some embodiments, the antibody or antigen-binding fragment thereof comprises a light chain variable region comprising an amino acid sequence having at least 80%, 85%, 90%, 95%, 96%, 97%, 98%, 99% identity to the amino acid sequence set forth in SEQ ID NO: 51. In some embodiments, the antibody or antigen-binding fragment thereof comprises an immunoglobulin heavy chain, comprising an amino acid sequence having at least 80%, 85%, 90%, 95%, 96%, 97%, 98%, 99% identity to the amino acid sequence set forth in SEQ ID NO: 55. In some embodiments, the antibody or antigen-binding fragment thereof comprises an immunoglobulin light chain, comprising an amino acid sequence having at least 80%, 85%, 90%, 95%, 96%, 97%, 98%, 99% identity to the amino acid sequence set forth in SEQ ID NO: 57.

In certain aspects, the present disclosure provides an antibody or antigen-binding fragment thereof comprises a heavy chain variable region comprising an amino acid sequence having at least 80%, 85%, 90%, 95%, 96%, 96%, 97%, 98%, 99% identity to the amino acid sequence set forth in SEQ ID NO: 49; and a light chain variable region comprising an amino acid sequence having at least 80%, 85%, 90%, 95%, 96%, 97%, 98%, 99% identity to the amino acid sequence set forth in SEQ ID NO: 51. In some cases, the antibody or antigen-binding fragment thereof specifically binds to canine programmed death protein 1 (PD-1).

In certain aspects, the present disclosure provides an antibody or antigen-binding fragment thereof comprises a heavy chain variable region comprising the amino acid sequence set forth in SEQ ID NO: 49; and a light chain variable region comprising the amino acid sequence set forth in SEQ ID NO: 51. In some cases, the antibody or antigen-binding fragment thereof specifically binds to canine programmed death protein 1 (PD-1).

In certain aspects, the present disclosure provides an antibody or antigen-binding fragment thereof comprises an immunoglobulin heavy chain, comprising an amino acid sequence having at least 80%, 85%, 90%, 95%, 96%, 97%, 98%, 99% identity to the amino acid sequence set forth in SEQ ID NO: 55, and an immunoglobulin light chain, comprising an amino acid sequence having at least 80%, 85%, 90%, 95%, 96%, 97%, 98%, 99% identity to the amino acid sequence set forth in SEQ ID NO: 57. In some cases, the antibody or antigen-binding fragment thereof specifically binds to canine programmed death protein 1 (PD-1).

In certain aspects, the present disclosure provides an antibody or antigen-binding fragment thereof comprises an immunoglobulin heavy chain, comprising the amino acid sequence set forth in SEQ ID NO: 55, and an immunoglobulin light chain, comprising the amino acid sequence set forth in SEQ ID NO: 57. In some cases, the antibody or antigen-binding fragment thereof specifically binds to canine programmed death protein 1 (PD-1).

Tolerable variations of the complementarity determining regions (CDR) sequences will be known to those of skill in the art. For example, in some embodiments, the isolated binding polypeptide comprises a complementarity determining region (HCDR or LCDR) that comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 1, 2, 3, 4, 5, 6, 27, 28, 29, 30, 31, 32, 43, 44, 45, 46, 47, or 48. in some embodiments the isolated binding polypeptide comprises a complementarity determining region (HCDR or LCDR) that comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 1, 2, 3, 4, 5, or 6. In some embodiments, the isolated binding polypeptide comprises a complementarity determining region (HCDR or LCDR) that comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 27, 28, 29, 30, 31, or 32. In some embodiments, the isolated binding polypeptide comprises a complementarity determining region (HCDR or LCDR) that comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 43, 44, 45, 46, 47, or 48. In some embodiments, the isolated binding polypeptide is an antibody or antigen-binding fragment thereof that specifically binds to canine programmed death protein 1 (PD-1).

In some embodiments, the isolated binding polypeptide binds a programmed death protein 1 (PD-1), for example, canine PD-1. In some embodiments, the binding polypeptide comprises an antibody or an antigen-binding fragment thereof. In some embodiments, the antibody or antigen-binding fragment thereof is an IgG, an IgM, an IgE, an IgA, or an IgD molecule. In some embodiments, the antibody or antigen-binding fragment thereof is derived from an IgG, an IgM, an IgE, an IgA, or an IgD molecule. In some embodiments, the antigen-binding fragment is selected from the group consisting of a full-length antibody, a Fab, a single-chain variable fragment (scFv), a single-domain antibody, sc(Fv)2, dsFv, Fab, Fab′, (Fab′)2 and a diabody. In further embodiments, the antibody is a full-length antibody. In yet further embodiments, the antibody or antigen-binding fragment is a canine or caninized antibody or antigen-binding fragment thereof. In some embodiments, the antibody or antigen-binding fragment is a canine antibody or an antigen-binding fragment thereof. In some embodiments, the antibody or antigen-binding fragment is a canine antibody. In some embodiments, the antibody or antigen-binding fragment is a canine IgG4 antibody. In some embodiments, the antibody or antigen-binding fragment is an scFv. In some embodiments, the antibody or antigen-binding fragment is a canine or caninized scFv. In some embodiments, the antibody or antigen-binding fragment is a canine scFv. In some embodiments, the specific binding of antibody or antigen-binding fragment thereof to cPD-1 disrupts the interaction of cPD-1 and a ligand of cPD-1 (e.g., canine programmed death-ligand 1). In some embodiments, the specific binding of antibody or antigen-binding fragment thereof to cPD-1 inhibits the interaction of cPD-1 and a ligand of cPD-1 (e.g., canine programmed death-ligand 1). In some embodiments, the specific binding of antibody or antigen-binding fragment thereof to cPD-1 inhibits a signaling pathway activated by interaction of cPD-1 and a ligand of cPD-1 (e.g., canine programmed death-ligand 1). In some embodiments, the specific binding of antibody or antigen-binding fragment thereof to cPD-1 inhibits cPD-1 signaling pathway.

In certain embodiments, the binding polypeptide comprises a heavy chain variable region comprising an amino acid sequence having at least 80%, 85%, 90%, 95%, 96%, 96%, 97%, 98%, 99% identity to the amino acid sequence of the heavy chain variable region set forth in SEQ ID NOs: 7, 9, 33, or 49. In certain embodiments, the binding polypeptide comprises a heavy chain variable region comprising the amino acid sequence set forth in SEQ ID NOs: 7, 9, 33, or 49. In certain embodiments, the binding polypeptide comprises a heavy chain variable region consisting of the amino acid sequences set forth in SEQ ID NO: 7, 9, 33, or 49.

In certain embodiments, the binding polypeptide comprises a light chain variable region comprising an amino acid sequence having at least 80%, 85%, 90%, 95%, 96%, 97%, 98%, 99% identity to the amino acid sequence set forth in SEQ ID NOs: 11, 35, or 51. In certain embodiments, the binding polypeptide comprises a light chain variable region comprising an amino acid sequence set forth in SEQ ID NOs: 11, 35, or 51. In certain embodiments, the binding polypeptide comprises a light chain variable region comprising an amino acid sequence set forth in SEQ ID NOs: 11, 35, or 51.

Also provided is an isolated binding polypeptide comprising a heavy chain variable region comprising the amino acid sequence set forth in SEQ ID NO: 7 or 9 and a light chain variable region comprising the amino acid sequence set forth in SEQ ID NO: 11.

Also provided is an isolated binding polypeptide comprising a heavy chain variable region comprising the amino acid sequence set forth in SEQ ID NO: 33 and a light chain variable region comprising the amino acid sequence set forth in SEQ ID NO: 35.

Also provided is an isolated binding polypeptide comprising a heavy chain variable region comprising the amino acid sequence set forth in SEQ ID NO: 49 and a light chain variable region comprising the amino acid sequence set forth in SEQ ID NO: 51.

In certain embodiments, the present disclosure includes an antibody that binds to the same epitope on canine PD-1 as an antibody of the present disclosure (i.e., antibodies that have the ability to cross-compete for binding to canine PD-1 with any of the antibodies of the present disclosure). In a preferred embodiment, the reference antibody for cross-competition studies can be one of the antibodies described herein (e.g., P3C6mut3.1, P3C6mut3.2, P4B1, or A6 in Table 1 and Example 1). For example, Biacore analysis, ELISA assays or flow cytometry can be used to demonstrate cross-competition with the antibodies of the current present disclosure. The ability of a test antibody to inhibit the binding of, for example, P3C6mut3.1 or P3C6mut3.2, to cPD-1 demonstrates that the test antibody can compete with P3C6mut3.1 or P3C6mut3.2 for binding to cPD-1 and thus is considered to bind to the same epitope of PD-1 as P3C6mut3.1 or P3C6mut3.2.

An antibody of the present disclosure can be prepared using an antibody having one or more of the V_{H} and/or V_{L} sequences or any fragments thereof disclosed herein as a starting material to engineer a modified antibody, which modified antibody can have altered properties as compared with the starting antibody. An antibody can be engineered by modifying one or more amino acids within one or both variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

Also provided is a canine antibody or antigen-binding fragment thereof that specifically binds to canine programmed death protein 1 (cPD-1). The canine antibody or antigen-binding fragment thereof can be generated from a canine antibody phage display library. The process for generating canine antibody or antigen-binding fragment thereof that specifically bind cPD-1 is illustrated in Example 1. In some cases, such canine antibody comprises any antigen-binding fragments or binding peptide disclosed herein. In some cases, the specific binding of antibody or antigen-binding fragment thereof to cPD-1 disrupts the interaction of cPD-1 and a ligand of cPD-1 (e.g., canine programmed death-ligand 1).

Also provided is a single-chain variable fragment (scFv) specifically binds to canine programmed death protein 1 (PD-1). In some cases, the scFv comprises an antigen-binding domain. In some cases, the specific binding of antibody or antigen-binding fragment thereof to cPD-1 disrupts the interaction of cPD-1 and a ligand of cPD-1 (e.g., canine programmed death-ligand 1).

As used herein, the term “single-chain variable fragment” or “scFv” is a fusion protein of the variable regions of the heavy (V_{H}) and light chains (V_{L}) of an immunoglobulin (e.g., mouse, canine, or human) covalently linked to form a V_{H}: V_{L} heterodimer. The heavy (V_{H}) and light chains (V_{L}) are either joined directly or joined by a peptide-encoding linker, which connects the N-terminus of the V_{H} with the C-terminus of the V_{L}, or the C-terminus of the V_{H} with the N-terminus of the V_{L}. In some embodiments, the antigen binding domain (e.g., PD-1 binding domain) comprises an scFv having the configuration from N-terminus to C-terminus, V_{H}-linker-V_{L}. In some embodiments, the antigen binding domain comprises an scFv having the configuration from N-terminus to C-terminus, V_{L}-linker-V_{H}. Those of skill in the art would be able to select the appropriate configuration for use in the present disclosure.

The linker is usually rich in glycine for flexibility, as well as serine or threonine for solubility. The linker can link the heavy chain variable region and the light chain variable region of the extracellular antigen-binding domain. Non-limiting examples of linkers are disclosed in Shen et al., Anal. Chem. 80 (6): 1910-1917 (2008) and WO 2014/087010, the contents of which are hereby incorporated by reference in their entireties. Various linker sequences are known in the art, including, without limitation, glycine serine (GS) linkers such as (GS)n, (GSGGS)n (SEQ ID NO: 65), (GGGS)n (SEQ ID NO: 66), and (GGGGS)n (SEQ ID NO: 67), where n represents an integer of at least 1. Exemplary linker sequences can comprise amino acid sequences including, without limitation, GGSG (SEQ ID NO: 68), GGSGG (SEQ ID NO: 69), GSGSG (SEQ ID NO:70), GSGGG (SEQ ID NO: 71), GGGSG (SEQ ID NO: 72), GSSSG (SEQ ID NO: 73), GGGGS (SEQ ID NO: 74), GGGGSGGGGSGGGGS (SEQ ID NO: 75), GGGSSRSSSSGGGGSGGGG (SEQ ID NO: 76) and the like. Those of skill in the art would be able to select the appropriate linker sequence for use in the present disclosure. In one embodiment, an scFv of the present disclosure comprises a heavy chain variable region (V_{H}) and a light chain variable region (V_{L}), wherein the V_{H} and V_{L} are connected by the linker sequence having the amino acid sequence GGGSSRSSSSGGGGSGGGG (SEQ ID NO: 76), which can be encoded by the nucleic acid sequence GGCGGTGGTTCCTCTAGATCTTCCTCCTCTGGTGG CGGTGGCTCGGGCGGTGGTGGG (SEQ ID NO: 77) in which an arginine residue, R, is present as a result of including a nucleotide sequence for the restriction endonuclease Xba I. The presence of restriction sites in the linker along with those flanking an scFv construct will be recognized by those skilled in the art to be useful for performing heavy chain/light chain “swapping” experiments for antibody optimization, if desired.

Despite removal of the constant regions and the introduction of a linker, scFv proteins retain the specificity of the original immunoglobulin. Single chain Fv polypeptide antibodies can be expressed from a nucleic acid comprising V_{H}- and V_{L}-encoding sequences as described by Huston et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27 (6): 455-51; Peter et al., J Cachexia Sarcopenia Muscle 2012 Aug. 12; Shieh et al., J Imunol 2009 183 (4): 2277-85; Giomarelli et al., Thromb Haemost 2007 97 (6): 955-63; Fife eta., J Clin Invst 2006 116 (8): 2252-61; Brocks et al., Immunotechnology 1997 3 (3): 173-84; Moosmayer et al., Ther Immunol 1995 2 (10:31-40). Agonistic scFvs having stimulatory activity have been described (see, e.g., Peter et al., J Bioi Chem 2003 25278 (38): 36740-7; Xie et al., Nat Biotech 1997 15 (8): 768-71; Ledbetter et al., Crit Rev Immunol 1997 17 (5-6): 427-55; Ho et al., BioChim Biophys Acta 2003 1638 (3): 257-66).

In certain embodiments, the antigen-binding domain of the scFv comprises a heavy chain variable region that comprises three heavy chain complementarity determining regions (HCDRs) and a light chain variable region that comprises three light chain complementarity determining regions (LCDRs). HCDR1 comprises the amino acid sequence (SEQ ID NOs: 1, 27, or 43), and/or HCDR2 comprises the amino acid sequence (SEQ ID NOs: 2, 28, or 44), and/or HCDR3 comprises the amino acid sequence (SEQ ID NO: 3, 29, or 45) and/or LCDR1 comprises the amino acid sequence (SEQ ID NOs: 4, 30, or 46), and/or LCDR2 comprises the amino acid sequence (SEQ ID NO: 5, 31, or 47), and/or LCDR3 comprises the amino acid sequence (SEQ ID NO: 6, 32, or 48). The heavy chain variable region and the light chain variable region are connected by a linker.

Also provided is a single-chain variable fragment (scFv) comprising a heavy chain variable region comprising the amino acid sequence set forth in SEQ ID NOs: 7, 9, 33, or 49 and a light chain variable region comprising the amino acid sequence set forth in SEQ ID NO: 11, 35, or 51. The heavy chain variable region and the light chain variable region are connected by a linker.

In another aspect, a single chain variable fragment (scFv) comprising an amino acid sequence set forth in SEQ ID NOs: 13, 15, 17, 37, or 53, is provided. In another aspect, a single chain variable fragment (scFv) consisting of an amino acid sequence set forth in SEQ ID NOs: 13, 15, 17, 37, or 53, is provided.

Tolerable variations of the scFv sequences will be known to those of skill in the art. For example, in some embodiments the scFv comprises an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 13, 15, 17, 37, or 53.

In another aspect, a full-length antibody comprising a heavy chain comprising an amino acid sequence set forth in SEQ ID NOs: 19, 21, 23, 39, or 55 and a light chain comprising an amino acid sequence set forth in SEQ ID NOs: 25, 41, or 57 is provided. In another aspect, a full-length antibody consisting of a heavy chain comprising an amino acid sequence set forth in SEQ ID NOs: 19, 21, 23, 39, or 55 and a light chain comprising an amino acid sequence set forth in SEQ ID NOs: 25, 41, or 57 is provided.

Tolerable variations of the full-length antibody sequences will be known to those of skill in the art. For example, in some embodiments the antibody comprises a heavy chain comprising an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 19, 21, 23, 39, or 55 and a light chain comprising an amino acid sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the amino acid sequences set forth in SEQ ID NOs: 25, 41, or 57.

In certain embodiments, the full-length heavy chains or light chains of the antibodies or antigen-binding fragments thereof of the present disclosure further comprise a leader sequence. In certain embodiments, the leader sequences comprise the amino acid sequence set forth in SEQ ID NO: 60. In certain embodiments, the leader sequence is encoded by the nucleotide sequence set forth in SEQ ID NO: 59.

In certain embodiments, the full-length heavy chains of the antibodies or antigen-binding fragments thereof of the present disclosure further comprise a linker and HA or hemagglutinin-tag on the carboxy terminus of the polypeptide. In certain embodiments, the heavy chain linker and HA tag comprise the amino acid sequence set forth in SEQ ID NO: 62. In certain embodiments, the heavy chain linker and HA tag are encoded by a nucleotide sequence set forth in SEQ ID NO: 61.

In certain embodiments, the scFvs of the present disclosure further comprises an IgG fragment and 6× HIS or histidine-hemagglutinin tag on the carboxy terminus of the polypeptide. In certain embodiments, the scFv IgG fragment-6× HIS-hemagglutinin tag comprises the amino acid sequence set forth in SEQ ID NO: 64. In certain embodiments, the scFv IgG fragment-6× HIS-hemagglutinin tag is encoded by a nucleic acid sequence set forth in SEQ ID NO: 63.

TABLE 1
Sequences used in the present disclosure

| SEQ | | | |
|---|---|---|---|
| ID | | | |
| NO: | Name | Type | Sequence |
| 1 | P3C6 HCDR1 | Protein | GFTFSKYG |
| 2 | P3C6 HCDR2 | Protein | IAANGGTY |
| 3 | P3C6 HCDR3 | Protein | RWGGAPFDY |
| 4 | P3C6 LCDR1 | Protein | QSVSSY |
| 5 | P3C6 LCDR2 | Protein | GTS |
| 6 | P3C6 LCDR3 | Protein | QQGLQYPIT |
| 7 | P3C6mut3.1 | Protein | EVQLVQSGGDVVKPGGSLRLSCMASGFTFSKYGMSWVRQSPGKG |
| | VH | | LQWVADIAANGGTYYTDAVKGRFTISRDNAKNTLYLQMNSLRAE |
| | | | DTAVYYCARWGGAPFDYWGQGTLVTVSS |
| 8 | P3C6mut3.1 | DNA | GAGGTGCAGCTGGTGCAGAGCGGAGGAGACGTGGTGAAGCCTGG |
| | VH | | AGGAAGCCTGAGGCTGAGCTGCATGGCAAGCGGATTCACCTTCA |
| | | | GCAAGTACGGAATGAGCTGGGTGAGGCAGAGCCCCGGCAAGGGC |
| | | | CTGCAGTGGGTGGCCGACATCGCCGCCAACGGCGGCACCTACTA |
| | | | CACCGACGCCGTGAAGGGCAGGTTCACCATCAGCAGGGACAACG |
| | | | CCAAGAACACCCTGTACCTGCAGATGAACAGCCTGAGGGCCGAG |
| | | | GACACCGCCGTGTACTACTGCGCCAGGTGGGGCGGCGCCCCCTT |
| | | | CGACTACTGGGGCCAGGGCACCCTGGTGACCGTGAGCAGC |
| 9 | P3C6mut3.2 | Protein | EVQLVQSGGDVVKPGGSLRLSCVASGFTFSKYGMSWVRQSPGKG |
| | VH | | LQWVADIAANGGTYYTDAVKGRFTISRDNAKNTLYLQMNSLRAE |
| | | | DTAVYYCARWGGAPFDYWGQGTLVTVSS |
| 10 | P3C6mut3.2 | DNA | GAGGTGCAGCTGGTGCAGAGCGGCGGCGACGTGGTGAAGCCCGG |
| | VH | | CGGCAGCCTGAGGCTGAGCTGCGTGGCCAGCGGCTTCACCTTCA |
| | | | GCAAGTACGGCATGAGCTGGGTGAGGCAGAGCCCCGGCAAGGGC |
| | | | CTGCAGTGGGTGGCCGACATCGCCGCCAACGGCGGCACCTACTA |
| | | | CACCGACGCCGTGAAGGGCAGGTTCACCATCAGCAGGGACAACG |
| | | | CCAAGAACACCCTGTACCTGCAGATGAACAGCCTGAGGGCCGAG |
| | | | GACACCGCCGTGTACTACTGCGCCAGGTGGGGCGGCGCCCCCTT |
| | | | CGACTACTGGGGCCAGGGCACCCTGGTGACTGTGAGCAGC |
| 11 | P3C6 VL | Protein | EIVMTQSPASLSLSQEEKVTITCRASQSVSSYLAWYQQKPGQAP |
| | | | KLLIYGTSNRATGVPSRFSGSGSGTDFSFTISSLEPEDVAVYYC |
| | | | QQGLQYPITFGKGTHLEIK |
| 12 | P3C6 VL | DNA | GAAATCGTGATGACCCAGTCTCCAGCCTCCCTCTCCTTGTCTCA |
| | | | GGAGGAAAAAGTCACCATCACCTGCCGGGCCAGTCAGAGTGTTA |
| | | | GCAGCTACTTAGCCTGGTACCAGCAAAAACCTGGGCAGGCTCCC |
| | | | AAGCTCCTCATCTATGGTACATCCAACAGGGCCACTGGTGTCCC |
| | | | ATCCCGGTTCAGTGGCAGTGGGTCTGGGACAGACTTCAGCTTCA |
| | | | CCATCAGCAGCCTGGAGCCTGAAGATGTTGCAGTTTATTACTGC |
| | | | CAGCAAGGTCTACAGTATCCGATCACCTTTGGCAAAGGGACACA |
| | | | TCTGGAGATTAAA |
| 13 | P3C6 scFv | Protein | EIVMTQSPASLSLSQEEKVTITCRASQSVSSYLAWYQQKPGQAP |
| | | | KLLIYGTSNRATGVPSRFSGSGSGTDFSFTISSLEPEDVAVYYC |
| | | | QQGLQYPITFGKGTHLEIKGGSSRSSSSGGGGSGGGGEVQLVQS |
| | | | GGDLVKPGGSLRLSCMASGFTFSKYGMSWVRQSPGKGLQWVADI |
| | | | AANGGTYYTDAVKGRFTISRDNAKNALYLQMDDLRAEDTAVYYC |
| | | | ARWGGAPFDYWGQGTLVTVSS |
| 14 | P3C6 scFv | DNA | GAAATCGTGATGACCCAGTCTCCAGCCTCCCTCTCCTTGTCTCA |
| | | | GGAGGAAAAAGTCACCATCACCTGCCGGGCCAGTCAGAGTGTTA |
| | | | GCAGCTACTTAGCCTGGTACCAGCAAAAACCTGGGCAGGCTCCC |
| | | | AAGCTCCTCATCTATGGTACATCCAACAGGGCCACTGGTGTCCC |
| | | | ATCCCGGTTCAGTGGCAGTGGGTCTGGGACAGACTTCAGCTTCA |
| | | | CCATCAGCAGCCTGGAGCCTGAAGATGTTGCAGTTTATTACTGC |
| | | | CAGCAAGGTCTACAGTATCCGATCACCTTTGGCAAAGGGACACA |
| | | | TCTGGAGATTAAAGGTGGTTCCTCTAGATCTTCCTCCTCTGGTG |
| | | | GCGGTGGCTCGGGCGGTGGTGGGGAGGTCCAGCTGGTGCAGTCT |
| | | | GGGGGAGACCTGGTGAAGCCTGGGGGGTCCTTGAGACTGTCCTG |
| | | | TATGGCCTCTGGATTCACCTTCAGTAAATATGGCATGAGCTGGG |
| | | | TCCGTCAGTCTCCAGGGAAGGGGCTGCAGTGGGTCGCAGATATT |
| | | | GCCGCTAATGGAGGCACATACTACACGGACGCTGTGAAGGGCCG |
| | | | ATTCACCATCTCCAGAGACAACGCCAAGAACGCGCTATATCTGC |
| | | | AGATGGACGATCTGAGAGCCGAGGACACGGCTGTCTATTATTGT |
| | | | GCGAGGTGGGGGGGGGCCCCCTTTGACTACTGGGGCCAGGGAAC |
| | | | CCTGGTCACCGTCTCCTCA |
| 15 | P3C6mut3.1 | Protein | EIVMTQSPASLSLSQEEKVTITCRASQSVSSYLAWYQQKPGQAP |
| | scFv | | KLLIYGTSNRATGVPSRFSGSGSGTDFSFTISSLEPEDVAVYYC |
| | | | QQGLQYPITFGKGTHLEIKGGSSRSSSSGGGGSGGGGEVQLVQS |
| | | | GGDVVKPGGSLRLSCMASGFTFSKYGMSWVRQSPGKGLQWVADI |
| | | | AANGGTYYTDAVKGRFTISRDNAKNTLYLQMNSLRAEDTAVYYC |
| | | | ARWGGAPFDYWGQGTLVTVSS |
| 16 | P3C6 mut3.1 | DNA | GAAATCGTGATGACCCAGTCTCCAGCCTCCCTCTCCTTGTCTCA |
| | scFv | | GGAGGAAAAAGTCACCATCACCTGCCGGGCCAGTCAGAGTGTTA |
| | | | GCAGCTACTTAGCCTGGTACCAGCAAAAACCTGGGCAGGCTCCC |
| | | | AAGCTCCTCATCTATGGTACATCCAACAGGGCCACTGGTGTCCC |
| | | | ATCCCGGTTCAGTGGCAGTGGGTCTGGGACAGACTTCAGCTTCA |
| | | | CCATCAGCAGCCTGGAGCCTGAAGATGTTGCAGTTTATTACTGC |
| | | | CAGCAAGGTCTACAGTATCCGATCACCTTTGGCAAAGGGACACA |
| | | | TCTGGAGATTAAAGGTGGTTCCTCTAGATCTTCCTCCTCTGGTG |
| | | | GCGGTGGCTCGGGCGGTGGTGGGGAGGTGCAGCTGGTGCAGAGC |
| | | | GGAGGAGACGTGGTGAAGCCTGGAGGAAGCCTGAGGCTGAGCTG |
| | | | CATGGCAAGCGGATTCACCTTCAGCAAGTACGGAATGAGCTGGG |
| | | | TGAGGCAGAGCCCCGGCAAGGGCCTGCAGTGGGTGGCCGACATC |
| | | | GCCGCCAACGGCGGCACCTACTACACCGACGCCGTGAAGGGCAG |
| | | | GTTCACCATCAGCAGGGACAACGCCAAGAACACCCTGTACCTGC |
| | | | AGATGAACAGCCTGAGGGCCGAGGACACCGCCGTGTACTACTGC |
| | | | GCCAGGTGGGGCGGCGCCCCCTTCGACTACTGGGGCCAGGGCAC |
| | | | CCTGGTGACCGTGAGCAGC |
| 17 | P3C6mut3.2 | Protein | EIVMTQSPASLSLSQEEKVTITCRASQSVSSYLAWYQQKPGQAP |
| | scFv | | KLLIYGTSNRATGVPSRFSGSGSGTDFSFTISSLEPEDVAVYYC |
| | | | QQGLQYPITFGKGTHLEIKGGSSRSSSSGGGGSGGGGEVQLVQS |
| | | | GGDVVKPGGSLRLSCVASGFTFSKYGMSWVRQSPGKGLQWVADI |
| | | | AANGGTYYTDAVKGRETISRDNAKNTLYLQMNSLRAEDTAVYYC |
| | | | ARWGGAPFDYWGQGTLVTVSS |
| 18 | P3C6mut3.2 | DNA | GAAATCGTGATGACCCAGTCTCCAGCCTCCCTCTCCTTGTCTCA |
| | scFv | | GGAGGAAAAAGTCACCATCACCTGCCGGGCCAGTCAGAGTGTTA |
| | | | GCAGCTACTTAGCCTGGTACCAGCAAAAACCTGGGCAGGCTCCC |
| | | | AAGCTCCTCATCTATGGTACATCCAACAGGGCCACTGGTGTCCC |
| | | | ATCCCGGTTCAGTGGCAGTGGGTCTGGGACAGACTTCAGCTTCA |
| | | | CCATCAGCAGCCTGGAGCCTGAAGATGTTGCAGTTTATTACTGC |
| | | | CAGCAAGGTCTACAGTATCCGATCACCTTTGGCAAAGGGACACA |
| | | | TCTGGAGATTAAAGGTGGTTCCTCTAGATCTTCCTCCTCTGGTG |
| | | | GCGGTGGCTCGGGCGGTGGTGGGGAGGTGCAGCTGGTGCAGAGC |
| | | | GGCGGCGACGTGGTGAAGCCCGGCGGCAGCCTGAGGCTGAGCTG |
| | | | CGTGGCCAGCGGCTTCACCTTCAGCAAGTACGGCATGAGCTGGG |
| | | | TGAGGCAGAGCCCCGGCAAGGGCCTGCAGTGGGTGGCCGACATC |
| | | | GCCGCCAACGGCGGCACCTACTACACCGACGCCGTGAAGGGCAG |
| | | | GTTCACCATCAGCAGGGACAACGCCAAGAACACCCTGTACCTGC |
| | | | AGATGAACAGCCTGAGGGCCGAGGACACCGCCGTGTACTACTGC |
| | | | GCCAGGTGGGGCGGCGCCCCCTTCGACTACTGGGGCCAGGGCAC |
| | | | CCTGGTGACTGTGAGCAGC |
| 19 | P3C6mut3.1 | Protein | EVQLVQSGGDVVKPGGSLRLSCMASGFTFSKYGMSWVRQSPGKG |
| | heavy chain | | LQWVADIAANGGTYYTDAVKGRFTISRDNAKNTLYLQMNSLRAE |
| | full-length | | DTAVYYCARWGGAPFDYWGQGTLVTVSSASTTAPSVEPLAPSCG |
| | IgG4 | | STSGSTVALACLVSGYFPEPVTVSWNSGSLTSGVHTFPSVLQSS |
| | | | GLYSLSSMVTVPSSRWPSETFTCNVVHPASNTKVDKPVPKESTC |
| | | | KCISPCPVPESLGGPSVFIFPPKPKDILRITRTPEITCVVLDLG |
| | | | REDPEVQISWFVDGKEVHTAKTQPREQQFNSTYRVVSVLPIEHQ |
| | | | DWLTGKEFKCRVNHIGLPSPIERTISKARGQAHQPSVYVLPPSP |
| | | | KELSSSDTVTLTCLIKDFFPPEIDVEWQSNGQPEPESKYHTTAP |
| | | | QLDEDGSYFLYSKLSVDKSRWQQGDPFTCAVMHEALQNHYTDLS |
| | | | LSHSPGK |
| 20 | P3C6mut3.1 | DNA | GAGGTGCAGCTGGTGCAGAGCGGAGGAGACGTGGTGAAGCCTGG |
| | heavy chain | | AGGAAGCCTGAGGCTGAGCTGCATGGCAAGCGGATTCACCTTCA |
| | full-length | | GCAAGTACGGAATGAGCTGGGTGAGGCAGAGCCCCGGCAAGGGC |
| | IgG4 | | CTGCAGTGGGTGGCCGACATCGCCGCCAACGGCGGCACCTACTA |
| | | | CACCGACGCCGTGAAGGGCAGGTTCACCATCAGCAGGGACAACG |
| | | | CCAAGAACACCCTGTACCTGCAGATGAACAGCCTGAGGGCCGAG |
| | | | GACACCGCCGTGTACTACTGCGCCAGGTGGGGCGGCGCCCCCTT |
| | | | CGACTACTGGGGCCAGGGCACCCTGGTGACCGTGAGCAGCGCTA |
| | | | GCACCACGGCCCCCTCGGTTTTCCCACTGGCCCCCAGCTGCGGG |
| | | | TCCACTTCCGGCTCCACGGTGGCCCTAGCCTGCCTGGTGTCAGG |
| | | | CTACTTCCCCGAGCCTGTAACTGTGTCCTGGAACTCCGGCTCCT |
| | | | TGACCAGCGGTGTGCACACCTTCCCGTCCGTCCTGCAGTCCTCA |
| | | | GGGCTCTACTCCCTCAGCAGCATGGTGACAGTGCCCTCCAGCAG |
| | | | GTGGCCCAGCGAGACCTTCACCTGCAACGTGGTCCACCCGGCCA |
| | | | GCAACACTAAAGTAGACAAGCCAGTGCCCAAAGAGTCCACCTGC |
| | | | AAGTGTATATCCCCATGCCCAGTCCCTGAATCACTGGGAGGGCC |
| | | | TTCGGTCTTCATCTTTCCCCCGAAACCCAAGGACATCCTCAGGA |
| | | | TTACCCGAACACCCGAGATCACCTGTGTGGTGTTAGATCTGGGC |
| | | | CGTGAGGACCCTGAGGTGCAGATCAGCTGGTTCGTGGATGGTAA |
| | | | GGAGGTGCACACAGCCAAGACGCAGCCTCGTGAGCAGCAGTTCA |
| | | | ACAGCACCTACCGTGTGGTCAGCGTCCTCCCCATTGAGCACCAG |
| | | | GACTGGCTCACCGGAAAGGAGTTCAAGTGCAGAGTCAACCACAT |
| | | | AGGCCTCCCGTCCCCCATCGAGAGGACCATCTCCAAAGCCAGAG |
| | | | GGCAAGCCCATCAGCCCAGTGTGTATGTCCTGCCACCATCCCCA |
| | | | AAGGAGTTGTCATCCAGTGACACGGTCACCCTGACCTGCCTGAT |
| | | | CAAAGACTTCTTCCCACCTGAGATTGATGTGGAGTGGCAGAGCA |
| | | | ATGGACAGCCAGAGCCTGAGAGCAAGTACCACACGACTGCACCC |
| | | | CAGCTGGACGAGGACGGGTCCTACTTCCTGTACAGCAAGCTCTC |
| | | | TGTGGACAAGAGCCGCTGGCAGCAGGGAGACCCCTTCACATGTG |
| | | | CGGTGATGCATGAAGCTCTACAGAACCACTACACAGATCTATCC |
| | | | CTCTCCCATTCTCCGGGTAAA |
| 21 | P3C6mut3.2 | Protein | EVQLVQSGGDVVKPGGSLRLSCVASGFTFSKYGMSWVRQSPGKG |
| | heavy chain | | LQWVADIAANGGTYYTDAVKGRFTISRDNAKNTLYLQMNSLRAE |
| | full-length | | DTAVYYCARWGGAPFDYWGQGTLVTVSSASTTAPSVFPLAPSCG |
| | IgG4 | | STSGSTVALACLVSGYFPEPVTVSWNSGSLTSGVHTFPSVLQSS |
| | | | GLYSLSSMVTVPSSRWPSETFTCNVVHPASNTKVDKPVPKESTC |
| | | | KCISPCPVPESLGGPSVFIFPPKPKDILRITRTPEITCVVLDLG |
| | | | REDPEVQISWFVDGKEVHTAKTQPREQQFNSTYRVVSVLPIEHQ |
| | | | DWLTGKEFKCRVNHIGLPSPIERTISKARGQAHQPSVYVLPPSP |
| | | | KELSSSDTVTLTCLIKDFFPPEIDVEWQSNGQPEPESKYHTTAP |
| | | | QLDEDGSYFLYSKLSVDKSRWQQGDPFTCAVMHEALQNHYTDLS |
| | | | LSHSPGK |
| 22 | P3C6mut3.2 | DNA | GAGGTGCAGCTGGTGCAGAGCGGCGGCGACGTGGTGAAGCCCGG |
| | heavy chain | | CGGCAGCCTGAGGCTGAGCTGCGTGGCCAGCGGCTTCACCTTCA |
| | full-length | | GCAAGTACGGCATGAGCTGGGTGAGGCAGAGCCCCGGCAAGGGC |
| | IgG4 | | CTGCAGTGGGTGGCCGACATCGCCGCCAACGGCGGCACCTACTA |
| | | | CACCGACGCCGTGAAGGGCAGGTTCACCATCAGCAGGGACAACG |
| | | | CCAAGAACACCCTGTACCTGCAGATGAACAGCCTGAGGGCCGAG |
| | | | GACACCGCCGTGTACTACTGCGCCAGGTGGGGCGGCGCCCCCTT |
| | | | CGACTACTGGGGCCAGGGCACCCTGGTGACTGTGAGCAGCGCTA |
| | | | GCACCACGGCCCCCTCGGTTTTCCCACTGGCCCCCAGCTGCGGG |
| | | | TCCACTTCCGGCTCCACGGTGGCCCTAGCCTGCCTGGTGTCAGG |
| | | | CTACTTCCCCGAGCCTGTAACTGTGTCCTGGAACTCCGGCTCCT |
| | | | TGACCAGCGGTGTGCACACCTTCCCGTCCGTCCTGCAGTCCTCA |
| | | | GGGCTCTACTCCCTCAGCAGCATGGTGACAGTGCCCTCCAGCAG |
| | | | GTGGCCCAGCGAGACCTTCACCTGCAACGTGGTCCACCCGGCCA |
| | | | GCAACACTAAAGTAGACAAGCCAGTGCCCAAAGAGTCCACCTGC |
| | | | AAGTGTATATCCCCATGCCCAGTCCCTGAATCACTGGGAGGGCC |
| | | | TTCGGTCTTCATCTTTCCCCCGAAACCCAAGGACATCCTCAGGA |
| | | | TTACCCGAACACCCGAGATCACCTGTGTGGTGTTAGATCTGGGC |
| | | | CGTGAGGACCCTGAGGTGCAGATCAGCTGGTTCGTGGATGGTAA |
| | | | GGAGGTGCACACAGCCAAGACGCAGCCTCGTGAGCAGCAGTTCA |
| | | | ACAGCACCTACCGTGTGGTCAGCGTCCTCCCCATTGAGCACCAG |
| | | | GACTGGCTCACCGGAAAGGAGTTCAAGTGCAGAGTCAACCACAT |
| | | | AGGCCTCCCGTCCCCCATCGAGAGGACCATCTCCAAAGCCAGAG |
| | | | GGCAAGCCCATCAGCCCAGTGTGTATGTCCTGCCACCATCCCCA |
| | | | AAGGAGTTGTCATCCAGTGACACGGTCACCCTGACCTGCCTGAT |
| | | | CAAAGACTTCTTCCCACCTGAGATTGATGTGGAGTGGCAGAGCA |
| | | | ATGGACAGCCAGAGCCTGAGAGCAAGTACCACACGACTGCACCC |
| | | | CAGCTGGACGAGGACGGGTCCTACTTCCTGTACAGCAAGCTCTC |
| | | | TGTGGACAAGAGCCGCTGGCAGCAGGGAGACCCCTTCACATGTG |
| | | | CGGTGATGCATGAAGCTCTACAGAACCACTACACAGATCTATCC |
| | | | CTCTCCCATTCTCCGGGTAAA |
| 23 | P3C6 heavy | Protein | EVQLVQSGGDLVKPGGSLRLSCMASGFTFSKYGMSWVRQSPGKG |
| | chain full- | | LQWVADIAANGGTYYTDAVKGRFTISRDNAKNALYLQMDDLRAE |
| | length IgG4 | | DTAVYYCARWGGAPFDYWGQGTLVTVSSASTTAPSVEPLAPSCG |
| | | | STSGSTVALACLVSGYFPEPVTVSWNSGSLTSGVHTFPSVLQSS |
| | | | GLYSLSSMVTVPSSRWPSETFTCNVVHPASNTKVDKPVPKESTC |
| | | | KCISPCPVPESLGGPSVFIFPPKPKDILRITRTPEITCVVLDLG |
| | | | REDPEVQISWFVDGKEVHTAKTQPREQQFNSTYRVVSVLPIEHQ |
| | | | DWLTGKEFKCRVNHIGLPSPIERTISKARGQAHQPSVYVLPPSP |
| | | | KELSSSDTVTLTCLIKDFFPPEIDVEWQSNGQPEPESKYHTTAP |
| | | | QLDEDGSYFLYSKLSVDKSRWQQGDPFTCAVMHEALQNHYTDLS |
| | | | LSHSPGK |
| 24 | P3C6 heavy | DNA | GAGGTCCAGCTGGTGCAGTCTGGGGGAGACCTGGTGAAGCCTGG |
| | chain full- | | GGGGTCCTTGAGACTGTCCTGTATGGCCTCTGGATTCACCTTCA |
| | length IgG4 | | GTAAATATGGCATGAGCTGGGTCCGTCAGTCTCCAGGGAAGGGG |
| | | | CTGCAGTGGGTCGCAGATATTGCCGCTAATGGAGGCACATACTA |
| | | | CACGGACGCTGTGAAGGGCCGATTCACCATCTCCAGAGACAACG |
| | | | CCAAGAACGCGCTATATCTGCAGATGGACGATCTGAGAGCCGAG |
| | | | GACACGGCTGTCTATTATTGTGCGAGGTGGGGGGGGGCCCCCTT |
| | | | TGACTACTGGGGCCAGGGAACCCTGGTCACCGTCTCCTCAGCTA |
| | | | GCACCACGGCCCCCTCGGTTTTCCCACTGGCCCCCAGCTGCGGG |
| | | | TCCACTTCCGGCTCCACGGTGGCCCTAGCCTGCCTGGTGTCAGG |
| | | | CTACTTCCCCGAGCCTGTAACTGTGTCCTGGAACTCCGGCTCCT |
| | | | TGACCAGCGGTGTGCACACCTTCCCGTCCGTCCTGCAGTCCTCA |
| | | | GGGCTCTACTCCCTCAGCAGCATGGTGACAGTGCCCTCCAGCAG |
| | | | GTGGCCCAGCGAGACCTTCACCTGCAACGTGGTCCACCCGGCCA |
| | | | GCAACACTAAAGTAGACAAGCCAGTGCCCAAAGAGTCCACCTGC |
| | | | AAGTGTATATCCCCATGCCCAGTCCCTGAATCACTGGGAGGGCC |
| | | | TTCGGTCTTCATCTTTCCCCCGAAACCCAAGGACATCCTCAGGA |
| | | | TTACCCGAACACCCGAGATCACCTGTGTGGTGTTAGATCTGGGC |
| | | | CGTGAGGACCCTGAGGTGCAGATCAGCTGGTTCGTGGATGGTAA |
| | | | GGAGGTGCACACAGCCAAGACGCAGCCTCGTGAGCAGCAGTTCA |
| | | | ACAGCACCTACCGTGTGGTCAGCGTCCTCCCCATTGAGCACCAG |
| | | | GACTGGCTCACCGGAAAGGAGTTCAAGTGCAGAGTCAACCACAT |
| | | | AGGCCTCCCGTCCCCCATCGAGAGGACCATCTCCAAAGCCAGAG |
| | | | GGCAAGCCCATCAGCCCAGTGTGTATGTCCTGCCACCATCCCCA |
| | | | AAGGAGTTGTCATCCAGTGACACGGTCACCCTGACCTGCCTGAT |
| | | | CAAAGACTTCTTCCCACCTGAGATTGATGTGGAGTGGCAGAGCA |
| | | | ATGGACAGCCAGAGCCTGAGAGCAAGTACCACACGACTGCACCC |
| | | | CAGCTGGACGAGGACGGGTCCTACTTCCTGTACAGCAAGCTCTC |
| | | | TGTGGACAAGAGCCGCTGGCAGCAGGGAGACCCCTTCACATGTG |
| | | | CGGTGATGCATGAAGCTCTACAGAACCACTACACAGATCTATCC |
| | | | CTCTCCCATTCTCCGGGTAAA |
| 25 | P3C6 light | Protein | EIVMTQSPASLSLSQEEKVTITCRASQSVSSYLAWYQQKPGQAP |
| | chain full- | | KLLIYGTSNRATGVPSRFSGSGSGTDFSFTISSLEPEDVAVYYC |
| | length | | QQGLQYPITFGKGTHLEIKRTDAQPAVYLFQPSPDQLHTGSASV |
| | | | VCLLNSFYPKDINVKWKVDGVIQDTGIQESVTEQDKDSTYSLSS |
| | | | TLTMSSTEYLSHELYSCEITHKSLPSTLIKSFQRSECQRVD |
| 26 | P3C6 light | DNA | GAAATCGTGATGACCCAGTCTCCAGCCTCCCTCTCCTTGTCTCA |
| | chain full- | | GGAGGAAAAAGTCACCATCACCTGCCGGGCCAGTCAGAGTGTTA |
| | length | | GCAGCTACTTAGCCTGGTACCAGCAAAAACCTGGGCAGGCTCCC |
| | | | AAGCTCCTCATCTATGGTACATCCAACAGGGCCACTGGTGTCCC |
| | | | ATCCCGGTTCAGTGGCAGTGGGTCTGGGACAGACTTCAGCTTCA |
| | | | CCATCAGCAGCCTGGAGCCTGAAGATGTTGCAGTTTATTACTGC |
| | | | CAGCAAGGTCTACAGTATCCGATCACCTTTGGCAAAGGGACACA |
| | | | TCTGGAGATTAAACGTACGGATGCCCAGCCAGCCGTCTATTTGT |
| | | | TCCAACCATCTCCAGACCAGTTACACACAGGAAGTGCCTCTGTT |
| | | | GTGTGTTTGCTGAATAGCTTCTACCCCAAAGACATCAATGTCAA |
| | | | GTGGAAAGTGGATGGTGTCATCCAAGACACAGGCATCCAGGAAA |
| | | | GTGTCACAGAGCAGGACAAGGACAGTACCTACAGCCTCAGCAGC |
| | | | ACCCTGACGATGTCCAGTACTGAGTACCTAAGTCATGAGTTGTA |
| | | | CTCCTGTGAGATCACTCACAAGAGCCTGCCCTCCACCCTCATCA |
| | | | AGAGCTTCCAAAGGAGCGAGTGTCAGAGAGTGGAC |
| 27 | P4B1 HCDR1 | Protein | GFTFNTYG |
| 28 | P4B1 HCDR2 | Protein | IANSGSTY |
| 29 | P4B1 HCDR3 | Protein | SGIWGDWFYY |
| 30 | P4B1 LCDR1 | Protein | SLTKYY |
| 31 | P4B1 LCDR2 | Protein | KDT |
| 32 | P4B1 LCDR3 | Protein | ESAVSADAVV |
| 33 | P4B1 VH | Protein | EVQLVQSGGDVVKPGGSLRLSCVASGFTFNTYGMNWVRQSPGKG |
| | | | LQWVAAIANSGSTYYADAVRGRFTISRDNAKNTLYLQMNSLRAE |
| | | | DAAIYYCASGIWGDWFYYWGQGTLVTVSS |
| 34 | P4B1 VH | DNA | GAGGTGCAGCTGGTGCAGTCTGGGGGAGACGTGGTGAAGCCTGG |
| | | | GGGGTCCTTGAGACTGTCCTGTGTGGCCTCTGGATTCACCTTTA |
| | | | ATACCTATGGCATGAACTGGGTCCGTCAGTCTCCTGGGAAGGGG |
| | | | CTGCAGTGGGTCGCGGCTATTGCCAATAGTGGAAGCACATATTA |
| | | | CGCAGACGCTGTGAGGGGCCGGTTCACCATCTCCAGAGACAACG |
| | | | CCAAGAACACACTTTATCTTCAGATGAACAGCCTGAGAGCCGAG |
| | | | GATGCGGCCATATATTACTGTGCGAGTGGTATTTGGGGTGACTG |
| | | | GTTCTACTACTGGGGCCAAGGGACCCTGGTCACTGTGTCCTCA |
| 35 | P4B1 VL | Protein | SSVLTQPPSVSVSLGQTATISCSGESLTKYYAQWFQQKPGQAPM |
| | | | LVIYKDTERPSGIPDRFSGSSSGNTHTLTISGARAEDEADYYCE |
| | | | SAVSADAVVEGGGTHLTVL |
| 36 | P4B1 VL | DNA | TCCAGTGTGCTGACTCAGCCTCCCTCGGTATCAGTGTCTCTGGG |
| | | | ACAGACAGCAACCATCTCCTGCTCTGGAGAGAGTCTGACTAAAT |
| | | | ATTATGCACAATGGTTCCAGCAGAAGCCAGGCCAAGCCCCTATG |
| | | | TTGGTCATATATAAGGACACTGAGCGGCCCTCTGGGATCCCTGA |
| | | | CCGATTCTCTGGCTCCAGTTCAGGAAACACACACACCCTGACCA |
| | | | TCAGCGGGGCTCGGGCCGAGGACGAGGCCGACTATTACTGCGAG |
| | | | TCAGCGGTCAGTGCTGATGCTGTTGTGTTCGGCGGGGGCACCCA |
| | | | TCTGACCGTCCTC |
| 37 | P4B1 scFv | Protein | SSVLTQPPSVSVSLGQTATISCSGESLTKYYAQWFQQKPGQAPM |
| | | | LVIYKDTERPSGIPDRFSGSSSGNTHTLTISGARAEDEADYYCE |
| | | | SAVSADAVVFGGGTHLTVLGGGSSRSSSSGGGGSGGGGEVQLVQ |
| | | | SGGDVVKPGGSLRLSCVASGFTFNTYGMNWVRQSPGKGLQWVAA |
| | | | IANSGSTYYADAVRGRFTISRDNAKNTLYLQMNSLRAEDAAIYY |
| | | | CASGIWGDWFYYWGQGTLVTVSS |
| 38 | P4B1 scFv | DNA | TCCAGTGTGCTGACTCAGCCTCCCTCGGTATCAGTGTCTCTGGG |
| | | | ACAGACAGCAACCATCTCCTGCTCTGGAGAGAGTCTGACTAAAT |
| | | | ATTATGCACAATGGTTCCAGCAGAAGCCAGGCCAAGCCCCTATG |
| | | | TTGGTCATATATAAGGACACTGAGCGGCCCTCTGGGATCCCTGA |
| | | | CCGATTCTCTGGCTCCAGTTCAGGAAACACACACACCCTGACCA |
| | | | TCAGCGGGGCTCGGGCCGAGGACGAGGCCGACTATTACTGCGAG |
| | | | TCAGCGGTCAGTGCTGATGCTGTTGTGTTCGGCGGGGGCACCCA |
| | | | TCTGACCGTCCTCGGTGGTGGTTCCTCTAGATCTTCCTCCTCTG |
| | | | GTGGCGGTGGCTCGGGCGGTGGTGGGGAGGTGCAGCTGGTGCAG |
| | | | TCTGGGGGAGACGTGGTGAAGCCTGGGGGGTCCTTGAGACTGTC |
| | | | CTGTGTGGCCTCTGGATTCACCTTTAATACCTATGGCATGAACT |
| | | | GGGTCCGTCAGTCTCCTGGGAAGGGGCTGCAGTGGGTCGCGGCT |
| | | | ATTGCCAATAGTGGAAGCACATATTACGCAGACGCTGTGAGGGG |
| | | | CCGGTTCACCATCTCCAGAGACAACGCCAAGAACACACTTTATC |
| | | | TTCAGATGAACAGCCTGAGAGCCGAGGATGCGGCCATATATTAC |
| | | | TGTGCGAGTGGTATTTGGGGTGACTGGTTCTACTACTGGGGCCA |
| | | | AGGGACCCTGGTCACTGTGTCCTCA |
| 39 | P4B1 heavy | Protein | EVQLVQSGGDVVKPGGSLRLSCVASGFTFNTYGMNWVRQSPGKG |
| | chain full- | | LQWVAAIANSGSTYYADAVRGRFTISRDNAKNTLYLQMNSLRAE |
| | length IgG4 | | DAAIYYCASGIWGDWFYYWGQGTLVTVSSASTTAPSVEPLAPSC |
| | | | GSTSGSTVALACLVSGYFPEPVTVSWNSGSLTSGVHTFPSVLQS |
| | | | SGLYSLSSMVTVPSSRWPSETFTCNVVHPASNTKVDKPVPKEST |
| | | | CKCISPCPVPESLGGPSVFIFPPKPKDILRITRTPEITCVVLDL |
| | | | GREDPEVQISWFVDGKEVHTAKTQPREQQENSTYRVVSVLPIEH |
| | | | QDWLTGKEFKCRVNHIGLPSPIERTISKARGQAHQPSVYVLPPS |
| | | | PKELSSSDTVTLTCLIKDFFPPEIDVEWQSNGQPEPESKYHTTA |
| | | | PQLDEDGSYFLYSKLSVDKSRWQQGDPFTCAVMHEALQNHYTDL |
| | | | SLSHSPGK |
| 40 | P4B1 heavy | DNA | GAGGTGCAGCTGGTGCAGTCTGGGGGAGACGTGGTGAAGCCTGG |
| | chain full- | | GGGGTCCTTGAGACTGTCCTGTGTGGCCTCTGGATTCACCTTTA |
| | length IgG4 | | ATACCTATGGCATGAACTGGGTCCGTCAGTCTCCTGGGAAGGGG |
| | | | CTGCAGTGGGTCGCGGCTATTGCCAATAGTGGAAGCACATATTA |
| | | | CGCAGACGCTGTGAGGGGCCGGTTCACCATCTCCAGAGACAACG |
| | | | CCAAGAACACACTTTATCTTCAGATGAACAGCCTGAGAGCCGAG |
| | | | GATGCGGCCATATATTACTGTGCGAGTGGTATTTGGGGTGACTG |
| | | | GTTCTACTACTGGGGCCAAGGGACCCTGGTCACTGTGTCCTCAG |
| | | | CTAGCACCACGGCCCCCTCGGTTTTCCCACTGGCCCCCAGCTGC |
| | | | GGGTCCACTTCCGGCTCCACGGTGGCCCTAGCCTGCCTGGTGTC |
| | | | AGGCTACTTCCCCGAGCCTGTAACTGTGTCCTGGAACTCCGGCT |
| | | | CCTTGACCAGCGGTGTGCACACCTTCCCGTCCGTCCTGCAGTCC |
| | | | TCAGGGCTCTACTCCCTCAGCAGCATGGTGACAGTGCCCTCCAG |
| | | | CAGGTGGCCCAGCGAGACCTTCACCTGCAACGTGGTCCACCCGG |
| | | | CCAGCAACACTAAAGTAGACAAGCCAGTGCCCAAAGAGTCCACC |
| | | | TGCAAGTGTATATCCCCATGCCCAGTCCCTGAATCACTGGGAGG |
| | | | GCCTTCGGTCTTCATCTTTCCCCCGAAACCCAAGGACATCCTCA |
| | | | GGATTACCCGAACACCCGAGATCACCTGTGTGGTGTTAGATCTG |
| | | | GGCCGTGAGGACCCTGAGGTGCAGATCAGCTGGTTCGTGGATGG |
| | | | TAAGGAGGTGCACACAGCCAAGACGCAGCCTCGTGAGCAGCAGT |
| | | | TCAACAGCACCTACCGTGTGGTCAGCGTCCTCCCCATTGAGCAC |
| | | | CAGGACTGGCTCACCGGAAAGGAGTTCAAGTGCAGAGTCAACCA |
| | | | CATAGGCCTCCCGTCCCCCATCGAGAGGACCATCTCCAAAGCCA |
| | | | GAGGGCAAGCCCATCAGCCCAGTGTGTATGTCCTGCCACCATCC |
| | | | CCAAAGGAGTTGTCATCCAGTGACACGGTCACCCTGACCTGCCT |
| | | | GATCAAAGACTTCTTCCCACCTGAGATTGATGTGGAGTGGCAGA |
| | | | GCAATGGACAGCCAGAGCCTGAGAGCAAGTACCACACGACTGCA |
| | | | CCCCAGCTGGACGAGGACGGGTCCTACTTCCTGTACAGCAAGCT |
| | | | CTCTGTGGACAAGAGCCGCTGGCAGCAGGGAGACCCCTTCACAT |
| | | | GTGCGGTGATGCATGAAGCTCTACAGAACCACTACACAGATCTA |
| | | | TCCCTCTCCCATTCTCCGGGTAAA |
| 41 | P4B1 light | Protein | SSVLTQPPSVSVSLGQTATISCSGESLTKYYAQWFQQKPGQAPM |
| | chain full- | | LVIYKDTERPSGIPDRFSGSSSGNTHTLTISGARAEDEADYYCE |
| | length | | SAVSADAVVFGGGTHLTVLGQPKASPSVTLFPPSSEELGANKAT |
| | | | LVCLISDFYPSGVTVAWKADGSPITQGVETTKPSKQSNNKYAAS |
| | | | SYLSLTPDKWKSHSSFSCLVTHEGSTVEKKVAPAECS |
| 42 | P4B1 light | DNA | TCCAGTGTGCTGACTCAGCCTCCCTCGGTATCAGTGTCTCTGGG |
| | chain full- | | ACAGACAGCAACCATCTCCTGCTCTGGAGAGAGTCTGACTAAAT |
| | length | | ATTATGCACAATGGTTCCAGCAGAAGCCAGGCCAAGCCCCTATG |
| | | | TTGGTCATATATAAGGACACTGAGCGGCCCTCTGGGATCCCTGA |
| | | | CCGATTCTCTGGCTCCAGTTCAGGAAACACACACACCCTGACCA |
| | | | TCAGCGGGGCTCGGGCCGAGGACGAGGCCGACTATTACTGCGAG |
| | | | TCAGCGGTCAGTGCTGATGCTGTTGTGTTCGGCGGGGGCACCCA |
| | | | TCTGACCGTCCTCGGTCAGCCCAAGGCCTCCCCCTCGGTCACAC |
| | | | TCTTCCCGCCCTCCTCTGAGGAGCTCGGCGCCAACAAGGCCACC |
| | | | CTGGTGTGCCTCATCAGCGACTTCTACCCCAGCGGCGTGACGGT |
| | | | GGCCTGGAAGGCAGACGGCAGCCCCATCACCCAGGGCGTGGAGA |
| | | | CCACCAAGCCCTCCAAGCAGAGCAACAACAAGTACGCGGCCAGC |
| | | | AGCTACCTGAGCCTGACGCCTGACAAGTGGAAATCTCACAGCAG |
| | | | CTTCAGCTGCCTGGTCACGCACGAGGGGAGCACCGTGGAGAAGA |
| | | | AGGTGGCCCCCGCAGAGTGCTCT |
| 43 | A6 HCDR1 | Protein | GFTFSSYD |
| 44 | A6 HCDR2 | Protein | ITTSGGST |
| 45 | A6 HCDR3 | Protein | AVGALGD |
| 46 | A6 LCDR1 | Protein | ASNIGLIG |
| 47 | A6 LCDR2 | Protein | SDG |
| 48 | A6 LCDR3 | Protein | QSIDSSLGDHFV |
| 49 | A6 VH | Protein | EVQLVESGGDLVKPGGSLRLSCVASGFTFSSYDMNWVRQSPGKG |
| | | | LQWVAAITTSGGSTYYTDAVKGRFTISRDNAKNTLYLQMNNLRA |
| | | | EDTAVYYCAVGALGDWGQGTLVTVSS |
| 50 | A6 VH | DNA | GAGGTACAGCTGGTGGAATCTGGGGGAGACTTGGTGAAGCCTGG |
| | | | GGGGTCCTTGAGACTGTCCTGTGTGGCCTCTGGATTCACCTTCA |
| | | | GTAGCTATGACATGAACTGGGTCCGTCAGTCTCCAGGGAAGGGG |
| | | | CTGCAGTGGGTCGCAGCTATTACCACTAGTGGAGGTAGTACATA |
| | | | CTACACAGACGCTGTGAAGGGCCGATTCACCATCTCCAGAGACA |
| | | | ATGCCAAGAACACACTATATCTGCAGATGAACAACCTGAGAGCC |
| | | | GAGGACACGGCTGTGTATTATTGTGCAGTAGGGGCTCTTGGTGA |
| | | | CTGGGGCCAGGGCACCCTGGTCACTGTCTCCTCA |
| 51 | A6 VL | Protein | QPVLTQPPSVSGALGQKVTISCSGSASNIGLIGAGWYQQVPGKA |
| | | | PKLLVYSDGNRPPQIPARFSGSNSGNSATLTIAGLQAEDEADYY |
| | | | CQSIDSSLGDHFVFGGGTHLTVL |
| 52 | A6 VL | DNA | CAGCCTGTGCTCACTCAGCCGCCCTCAGTGTCGGGGGCCCTTGG |
| | | | CCAGAAGGTCACCATCTCCTGCTCTGGAAGCGCGAGCAACATCG |
| | | | GTCTTATTGGTGCGGGCTGGTACCAACAAGTCCCAGGAAAGGCC |
| | | | CCTAAACTCCTCGTGTACAGTGATGGGAATCGACCGCCACAAAT |
| | | | CCCTGCCCGGTTCTCCGGCTCCAACTCTGGCAACTCAGCCACCC |
| | | | TGACTATCGCTGGGCTTCAGGCTGAGGACGAGGCTGATTATTAC |
| | | | TGTCAGTCTATTGATTCCAGCCTTGGTGATCATTTTGTGTTCGG |
| | | | CGGAGGCACCCATCTGACCGTCCTC |
| 53 | A6 scFv | Protein | QPVLTQPPSVSGALGQKVTISCSGSASNIGLIGAGWYQQVPGKA |
| | | | PKLLVYSDGNRPPQIPARFSGSNSGNSATLTIAGLQAEDEADYY |
| | | | CQSIDSSLGDHFVFGGGTHLTVLGGGSSRSSSSGGGGSGGGGEV |
| | | | QLVESGGDLVKPGGSLRLSCVASGFTFSSYDMNWVRQSPGKGLQ |
| | | | WVAAITTSGGSTYYTDAVKGRFTISRDNAKNTLYLQMNNLRAED |
| | | | TAVYYCAVGALGDWGQGTLVTVSS |
| 54 | A6 scFv | DNA | CAGCCTGTGCTCACTCAGCCGCCCTCAGTGTCGGGGGCCCTTGG |
| | | | CCAGAAGGTCACCATCTCCTGCTCTGGAAGCGCGAGCAACATCG |
| | | | GTCTTATTGGTGCGGGCTGGTACCAACAAGTCCCAGGAAAGGCC |
| | | | CCTAAACTCCTCGTGTACAGTGATGGGAATCGACCGCCACAAAT |
| | | | CCCTGCCCGGTTCTCCGGCTCCAACTCTGGCAACTCAGCCACCC |
| | | | TGACTATCGCTGGGCTTCAGGCTGAGGACGAGGCTGATTATTAC |
| | | | TGTCAGTCTATTGATTCCAGCCTTGGTGATCATTTTGTGTTCGG |
| | | | CGGAGGCACCCATCTGACCGTCCTCGGTGGTGGTTCCTCTAGAT |
| | | | CTTCCTCCTCTGGCGGCGGTGGCTCGGGCGGTGGTGGGGAGGTA |
| | | | CAGCTGGTGGAATCTGGGGGAGACTTGGTGAAGCCTGGGGGGTC |
| | | | CTTGAGACTGTCCTGTGTGGCCTCTGGATTCACCTTCAGTAGCT |
| | | | ATGACATGAACTGGGTCCGTCAGTCTCCAGGGAAGGGGCTGCAG |
| | | | TGGGTCGCAGCTATTACCACTAGTGGAGGTAGTACATACTACAC |
| | | | AGACGCTGTGAAGGGCCGATTCACCATCTCCAGAGACAATGCCA |
| | | | AGAACACACTATATCTGCAGATGAACAACCTGAGAGCCGAGGAC |
| | | | ACGGCTGTGTATTATTGTGCAGTAGGGGCTCTTGGTGACTGGGG |
| | | | CCAGGGCACCCTGGTCACTGTCTCCTCA |
| 55 | A6 heavy | Protein | EVQLVESGGDLVKPGGSLRLSCVASGFTFSSYDMNWVRQSPGKG |
| | chain full- | | LQWVAAITTSGGSTYYTDAVKGRFTISRDNAKNTLYLQMNNLRA |
| | length IgG4 | | EDTAVYYCAVGALGDWGQGTLVTVSSASTTAPSVFPLAPSCGST |
| | | | SGSTVALACLVSGYFPEPVTVSWNSGSLTSGVHTFPSVLQSSGL |
| | | | YSLSSMVTVPSSRWPSETFTCNVVHPASNTKVDKPVPKESTCKC |
| | | | ISPCPVPESLGGPSVFIFPPKPKDILRITRTPEITCVVLDLGRE |
| | | | DPEVQISWFVDGKEVHTAKTQPREQQENSTYRVVSVLPIEHQDW |
| | | | LTGKEFKCRVNHIGLPSPIERTISKARGQAHQPSVYVLPPSPKE |
| | | | LSSSDTVTLTCLIKDFFPPEIDVEWQSNGQPEPESKYHTTAPQL |
| | | | DEDGSYFLYSKLSVDKSRWQQGDPFTCAVMHEALQNHYTDLSLS |
| | | | HSPGK |
| 56 | A6 heavy | DNA | GAGGTACAGCTGGTGGAATCTGGGGGAGACTTGGTGAAGCCTGG |
| | chain full- | | GGGGTCCTTGAGACTGTCCTGTGTGGCCTCTGGATTCACCTTCA |
| | length IgG4 | | GTAGCTATGACATGAACTGGGTCCGTCAGTCTCCAGGGAAGGGG |
| | | | CTGCAGTGGGTCGCAGCTATTACCACTAGTGGAGGTAGTACATA |
| | | | CTACACAGACGCTGTGAAGGGCCGATTCACCATCTCCAGAGACA |
| | | | ATGCCAAGAACACACTATATCTGCAGATGAACAACCTGAGAGCC |
| | | | GAGGACACGGCTGTGTATTATTGTGCAGTAGGGGCTCTTGGTGA |
| | | | CTGGGGCCAGGGCACCCTGGTCACTGTCTCCTCAGCTAGCACCA |
| | | | CGGCCCCCTCGGTTTTCCCACTGGCCCCCAGCTGCGGGTCCACT |
| | | | TCCGGCTCCACGGTGGCCCTAGCCTGCCTGGTGTCAGGCTACTT |
| | | | CCCCGAGCCTGTAACTGTGTCCTGGAACTCCGGCTCCTTGACCA |
| | | | GCGGTGTGCACACCTTCCCGTCCGTCCTGCAGTCCTCAGGGCTC |
| | | | TACTCCCTCAGCAGCATGGTGACAGTGCCCTCCAGCAGGTGGCC |
| | | | CAGCGAGACCTTCACCTGCAACGTGGTCCACCCGGCCAGCAACA |
| | | | CTAAAGTAGACAAGCCAGTGCCCAAAGAGTCCACCTGCAAGTGT |
| | | | ATATCCCCATGCCCAGTCCCTGAATCACTGGGAGGGCCTTCGGT |
| | | | CTTCATCTTTCCCCCGAAACCCAAGGACATCCTCAGGATTACCC |
| | | | GAACACCCGAGATCACCTGTGTGGTGTTAGATCTGGGCCGTGAG |
| | | | GACCCTGAGGTGCAGATCAGCTGGTTCGTGGATGGTAAGGAGGT |
| | | | GCACACAGCCAAGACGCAGCCTCGTGAGCAGCAGTTCAACAGCA |
| | | | CCTACCGTGTGGTCAGCGTCCTCCCCATTGAGCACCAGGACTGG |
| | | | CTCACCGGAAAGGAGTTCAAGTGCAGAGTCAACCACATAGGCCT |
| | | | CCCGTCCCCCATCGAGAGGACCATCTCCAAAGCCAGAGGGCAAG |
| | | | CCCATCAGCCCAGTGTGTATGTCCTGCCACCATCCCCAAAGGAG |
| | | | TTGTCATCCAGTGACACGGTCACCCTGACCTGCCTGATCAAAGA |
| | | | CTTCTTCCCACCTGAGATTGATGTGGAGTGGCAGAGCAATGGAC |
| | | | AGCCAGAGCCTGAGAGCAAGTACCACACGACTGCACCCCAGCTG |
| | | | GACGAGGACGGGTCCTACTTCCTGTACAGCAAGCTCTCTGTGGA |
| | | | CAAGAGCCGCTGGCAGCAGGGAGACCCCTTCACATGTGCGGTGA |
| | | | TGCATGAAGCTCTACAGAACCACTACACAGATCTATCCCTCTCC |
| | | | CATTCTCCGGGTAAA |
| 57 | A6 light chain | Protein | QPVLTQPPSVSGALGQKVTISCSGSASNIGLIGAGWYQQVPGKA |
| | full-length | | PKLLVYSDGNRPPQIPARFSGSNSGNSATLTIAGLQAEDEADYY |
| | IgG4 | | CQSIDSSLGDHFVFGGGTHLTVLGQPKASPSVTLFPPSSEELGA |
| | | | NKATLVCLISDFYPSGVTVAWKADGSPITQGVETTKPSKQSNNK |
| | | | YAASSYLSLTPDKWKSHSSFSCLVTHEGSTVEKKVAPAECS |
| 58 | A6 light chain | DNA | CAGCCTGTGCTCACTCAGCCGCCCTCAGTGTCGGGGGCCCTTGG |
| | full-length | | CCAGAAGGTCACCATCTCCTGCTCTGGAAGCGCGAGCAACATCG |
| | IgG4 | | GTCTTATTGGTGCGGGCTGGTACCAACAAGTCCCAGGAAAGGCC |
| | | | CCTAAACTCCTCGTGTACAGTGATGGGAATCGACCGCCACAAAT |
| | | | CCCTGCCCGGTTCTCCGGCTCCAACTCTGGCAACTCAGCCACCC |
| | | | TGACTATCGCTGGGCTTCAGGCTGAGGACGAGGCTGATTATTAC |
| | | | TGTCAGTCTATTGATTCCAGCCTTGGTGATCATTTTGTGTTCGG |
| | | | CGGAGGCACCCATCTGACCGTCCTAGGTCAGCCCAAGGCCTCCC |
| | | | CCTCGGTCACACTCTTCCCGCCCTCCTCTGAGGAGCTCGGCGCC |
| | | | AACAAGGCCACCCTGGTGTGCCTCATCAGCGACTTCTACCCCAG |
| | | | CGGCGTGACGGTGGCCTGGAAGGCAGACGGCAGCCCCATCACCC |
| | | | AGGGCGTGGAGACCACCAAGCCCTCCAAGCAGAGCAACAACAAG |
| | | | TACGCGGCCAGCAGCTACCTGAGCCTGACGCCTGACAAGTGGAA |
| | | | ATCTCACAGCAGCTTCAGCTGCCTGGTCACGCACGAGGGGAGCA |
| | | | CCGTGGAGAAGAAGGTGGCCCCCGCAGAGTGCTCT |
| 59 | Leader | DNA | ATGTACAGGATGCAACTCCTGTCTTGCATTGCACTAAGTCTTGC |
| | sequence for | | ACTTGTCACGAATTCG |
| | full-length | | |
| | HC/LC | | |
| 60 | Leader | Protein | MYRMQLLSCIALSLALVINS |
| | sequence for | | |
| | full-length | | |
| | HC/LC | | |
| 61 | Linker and | DNA | GGCGGAGGCTCCGGAGGCGGATCTTACCCATACGATGTTCCAGA |
| | HA tag | | TTACGCT |
| 62 | Linker and | Protein | GGGSGGGSYPYDVPDYA |
| | HA tag | | |
| 63 | IgG fragment | DNA | GCCTCCACCACGGCCCCCTCGGTCACTAGTGGCCAGGCCGGCCA |
| | and 6x HIS | | GCACCATCACCATCACCATGGCGCATACCCGTACGACGTTCCGG |
| | tag for scFvs | | ACTACGCTTCT |
| 64 | IgG fragment | Protein | ASTTAPSVTSGQAGQHHHHHHGAYPYDVPDYAS |
| | and 6x HIS | | |
| | tag for scFvs | | |

### PD-1 Blockade

In some aspects, the binding polypeptides, the antibody or antigen-binding fragments thereof described in the present disclosure can bind to canine PD-1 and therefore can block canine PD-1 interaction with its natural ligands and any downstream signaling pathways associated with such interaction. In some cases, the antibody or antigen-binding fragments described herein can bind to a region on canine PD-1 that is the same region to which a natural ligand of canine PD-1 recognizes. In some cases, the antibody or antigen-binding fragments described herein can bind to a region on canine PD-1 that is a different region to which a natural ligand of canine PD-1 recognizes. In some cases, the binding of antibody or antigen-binding fragments described herein to canine PD-1 can hinder the region to which a natural ligand of canine PD-1 recognizes. In some cases, the binding of antibody or antigen-binding fragments described herein to canine PD-1 can prevent the interaction of canine PD-1 and its natural ligands, for example, canine PD-L1. In some cases, the binding of antibody or antigen-binding fragments described herein to canine PD-1 can inhibit canine PD-1 signaling pathway that can be activated by a natural ligand of canine PD-1, such as PD-L1.

Programmed death protein 1 (PD-1 or PD1), also known as “programmed cell death protein 1” and CD279, is a member of the immunoglobulin superfamily of transmembrane receptors that includes both stimulatory and regulatory receptors. The intracellular domain of PD-1 contains two phosphorylation sites located in an immunoreceptor tyrosine-based inhibitory motif (ITIM) that deliver signals which oppose T cell activation via the T cell receptor (TCR). While not expressed on naïve, resting CD4+ and CD8+ T cells, PD-1 is strongly up-regulated on the surface of activated T cells. The ligands for PD-1 are PD-L1 and PD-L2 which are part of the B7 family of receptors, and both deliver a negative signal to the T cell. Expression of both PD-1 ligands can be constitutive or can be induced in a variety of cell types. PD-L1 is generally expressed on antigen-presenting cells such as macrophages and dendritic cells in response to signals such as lipopolysaccharide (LPS), IFNγ, and GM-CSF but can also be found on peripheral, non-lymphoid cells including microvascular endothelial cells as well as organ tissues such as heart and lung. In particular, IFNγ signaling results in PD-L1 expression, where it contributes to limiting inflammation. PD-L2 is largely expressed by antigen-presenting cells, though it can also be found on a wide variety of tissues including the GI tract, skeletal muscle, and pancreas.

PD-L1 and PD-L2 can both be strongly expressed by many cancers as a mechanism for evading immune recognition and killing, effectively blunting T cell responses directed against them. Indeed, high expression of PD-1 ligands often correlates with poor prognosis and more aggressive disease. Likewise, IFNγ signaling in the tumor microenvironment, for instance as generated by the presence of anti-tumor cytotoxic CD8+ T cells, can stimulate the expression of PD-L1 by many types of tumor cells. Tumor-associated expression of PD-1 on tumor-infiltrating T cells and PD-L1 on tumor cells has been identified in a number of primary cancers including, but not limited to cervical cancer, lung cancers, liver cancers, ovarian cancers, cancers of the skin including melanoma and squamous cell carcinoma, colon cancer, bladder cancer, breast cancer, kidney cancer, esophageal cancer, stomach cancer, pancreatic cancers, head and neck cancers, among others.

The blockade and inhibition of the PD-1 signaling pathway was used as a treatment strategy for chronic infections, where suppressing PD-1 can reverse or prevent T cell exhaustion and maintained cytotoxic function. Chronic infections include viral infections. Such infections are often characterized by the exhaustion, or functional impairment, of CD8+ T cells which contributes to the failure of the subject to resolve the infection. The blockade of PD-1 in animal models (e.g., canine or pet dogs) of chronic infection can prevent T exhaustion and maintain both the number and function of responding T cells and can improve pathogen clearance and subject survival.

The fully canine anti-PD-1 disclosed herein can be used to inhibit the PD-1 signaling pathway and thereof serve as an anti-tumor therapy in dogs and an important comparative tool for translational research in human immune-oncology.

### Nucleic Acids and Expression Vectors

The present disclosure provides an isolated nucleic acid encoding a polypeptide. The nucleic acid of the present disclosure can comprise a polynucleotide sequence encoding any one of the binding polypeptides, scFv, antibodies or any fragments thereof disclosed herein.

One aspect of the present disclosure includes an isolated nucleic acid encoding a binding polypeptide comprising an antigen-binding domain that specifically binds canine programmed death protein 1 (PD-1).

In certain embodiments, the nucleic acid comprises an antigen binding domain comprising a heavy chain variable region that comprises three heavy chain complementarity determining regions (HCDRs) and a light chain variable region that comprises three light chain complementarity determining regions (LCDRs). HCDR1 comprises the amino acid sequences (SEQ ID NOs: 1, 27, or 43), and/or HCDR2 comprises the amino acid sequences (SEQ ID NOs: 2, 28, or 44), and/or HCDR3 comprises the amino acid sequences (SEQ ID NO: 3, 29, or 45) and/or LCDR1 comprises the amino acid sequences (SEQ ID NOs: 4, 30, or 46), and/or LCDR2 comprises the amino acid sequences (SEQ ID NO: 5, 31, or 47), and/or LCDR3 comprises the amino acid sequences (SEQ ID NO: 6, 32, or 48).

In certain embodiments, the binding polypeptide comprises an antibody or an antigen-binding fragment thereof. In certain embodiments, the antigen-binding fragment is selected from the group consisting of a Fab, a single-chain variable fragment (scFv), a single-domain antibody, sc(Fv)2, dsFv, Fab, Fab′, (Fab′)2 and a diabody. In certain embodiments, the antibody is a full-length antibody. In certain embodiments, the antibody or antigen-binding fragment is canine or caninized antibody or a fragment thereof.

In certain embodiments, the heavy chain variable region is encoded by a nucleic acid comprising a polynucleotide sequence having at least 80%, 85%, 90%, 95%, 96%, 96%, 97%, 98%, 99% identity to SEQ ID NOs: 8, 10, 34, or 50.

In certain embodiments, the heavy chain variable region is encoded by a nucleic acid comprising the polynucleotide sequence set forth in SEQ ID NOs: 8, 10, 34, or 50.

In certain embodiments, the heavy chain variable region is encoded by a nucleic acid consisting of the polynucleotide sequence set forth in SEQ ID NO: 8, 10, 34, or 50.

In certain embodiments, the light chain variable region is encoded by a nucleic acid comprising a polynucleotide sequence having at least 80%, 85%, 90%, 95%, 96%, 96%, 97%, 98%, 99% identity to the amino acid sequence of the light chain variable region set forth in SEQ ID NOs: 12, 36, or 52. In certain embodiments, the light chain variable region is encoded by a nucleic acid comprising the polynucleotide sequence set forth in SEQ ID NOs: 12, 36, or 52. In certain embodiments, the light chain variable region is encoded by a nucleic acid consisting of a polynucleotide sequence set forth in SEQ ID NOs: 12, 36, or 52.

Also provided is an isolated nucleic acid encoding a binding polypeptide comprising a heavy chain variable region encoded by a nucleic acid sequence comprising the polynucleotide sequence set forth in SEQ ID NO: 8 or 10, and a light chain variable region encoded by a nucleic acid sequence comprising the polynucleotide sequence set forth in SEQ ID NO: 12.

Also provided is an isolated nucleic acid encoding a binding polypeptide comprising a heavy chain variable region encoded by a nucleic acid sequence comprising the polynucleotide sequence set forth in SEQ ID NO: 34, and a light chain variable region encoded by a nucleic acid sequence comprising the polynucleotide sequence set forth in SEQ ID NO: 36.

Also provided is an isolated nucleic acid encoding a binding polypeptide comprising a heavy chain variable region encoded by a nucleic acid sequence comprising the polynucleotide sequence set forth in SEQ ID NO: 50, and a light chain variable region encoded by a nucleic acid sequence comprising the polynucleotide sequence set forth in SEQ ID NO: 52.

Also provided is an isolated nucleic acid encoding a single-chain variable fragment (scFv) comprising a heavy chain variable region that comprises three heavy chain complementarity determining regions (HCDRs) and a light chain variable region that comprises three light chain complementarity determining regions (LCDRs). HCDR1 comprises the amino acid sequence (SEQ ID NOs: 1, 27, and 43), and/or HCDR2 comprises the amino acid sequence (SEQ ID NO: 2, 28, or 44), and/or HCDR3 comprises the amino acid sequence (SEQ ID NO: 3, 29, and 45), and/or LCDR1 comprises the amino acid sequence (SEQ ID NO: 4, 30, or 46, and/or LCDR2 comprises the amino acid sequence (SEQ ID NO: 5, 31, or 47), and/or LCDR3 comprises the amino acid sequence (SEQ ID NO: 6, 32, or 48).

Also provided is an isolated nucleic acid encoding a single-chain variable fragment (scFv) comprising a heavy chain variable region comprising a nucleotide sequence set forth in SEQ ID NO: 8 or 10; and/or a light chain variable region comprising a nucleotide sequence set forth in SEQ ID NO: 12. The heavy chain variable region and the light chain variable region are connected by a linker.

Also provided is an isolated nucleic acid encoding a single-chain variable fragment (scFv) comprising a polynucleotide sequence set forth in SEQ ID NOs: 14, 16, 18, 38, or 54. Also provided is an isolated nucleic acid encoding a single-chain variable fragment (scFv) consisting of a polynucleotide sequence set forth in SEQ ID NOs: 14, 16, 18, 38, or 54.

Also provided is an isolated nucleic acid encoding a full-length antibody comprising a heavy chain polynucleotide sequence set forth in SEQ ID NOs: 20, 22, 40, or 56 and a light chain polynucleotide sequence set forth in SEQ ID NOs: 26, 42, or 58. Also provided is an isolated nucleic acid encoding a full-length antibody consisting of a heavy chain polynucleotide sequence set forth in SEQ ID NOs: 20, 22, 40, or 56 and a light chain polynucleotide sequence set forth in SEQ ID NOs: 26, 42, or 58.

Tolerable variations of the nucleic acid sequences will be known to those of skill in the art. For example, in some embodiments the nucleic acid encoding a full-length heavy chain comprises a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the nucleotide sequences set forth in SEQ ID NOs: 20, 22, 40, or 56, and the nucleic acid encoding a full-length light chain comprises a nucleotide sequence that has at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to any of the nucleotide sequences set forth in SEQ ID NOs: 26, 42, or 58.

In certain embodiments, a nucleic acid of the present disclosure comprises a first polynucleotide sequence and a second polynucleotide sequence. The first and second polynucleotide sequence can be connected by a linker. For example, in certain embodiments the heavy chain variable region and the light chain variable region of an scFv are connected by a linker. In certain embodiments, the nucleic acid comprises from 5′ to 3′ the first polynucleotide sequence, the linker, and the second polynucleotide sequence. In certain embodiments, the nucleic acid comprises from 5′ to 3′ the second polynucleotide sequence, the linker, and the first polynucleotide sequence.

Another aspect of the present disclosure provides a vector comprising any one of the isolated nucleic acids disclosed herein. In certain embodiments, the vector is selected from the group consisting of a DNA vector, an RNA vector, a plasmid, a lentiviral vector, an adenoviral vector, an adeno-associated viral vector, and a retroviral vector. In certain embodiments, the vector is an expression vector.

Also provided is a host cell comprising any of the vectors or nucleic acids disclosed herein. The host cell can be of eukaryotic, prokaryotic, mammalian, or bacterial origin. Non-limiting examples of cells that can be used to express the anti-cPD-1 antibodies or antigen-binding fragments of scFv disclosed herein include Human embryonic kidney (HEK) cell lines (e.g., HEK293), Chinese hamster ovary (CHO) cell lines, Baby hamster kidney (BHK) cell lines, COS cell lines, Madin Darby canine kidney (MDCK) cell line, and HeLa cell lines. In some cases, the host cell is a Chinese Hamster Ovary cell.

A method of producing an antibody, a binding polypeptide or scFv that binds to canine PD-1 is also provided herein, wherein the method comprises culturing the host cell. In some embodiments, the method further comprises incubating the host cell in a cell culture medium under conditions sufficient to allow expression and secretion of the anti-cPD-1 antibody or antigen-binding fragment thereof or the scFv described herein.

In some embodiments, a nucleic acid of the present disclosure can be operably linked to a transcriptional control element, e.g., a promoter, and enhancer, etc. Suitable promoter and enhancer elements are known to those of skill in the art.

In certain embodiments, the nucleic acid is in operable linkage with a promoter. In certain embodiments, the promoter is a phosphoglycerate kinase-1 (PGK) promoter.

For expression in a bacterial cell, suitable promoters include, but are not limited to, lacI, lacZ, T3, T7, gpt, lambda P and trc. For expression in a eukaryotic cell, suitable promoters include, but are not limited to, light and/or heavy chain immunoglobulin gene promoter and enhancer elements; cytomegalovirus immediate early promoter; herpes simplex virus thymidine kinase promoter; early and late SV40 promoters; promoter present in long terminal repeats from a retrovirus; mouse metallothionein-I promoter; and various art-known tissue specific promoters. Suitable reversible promoters, including reversible inducible promoters are known in the art. Such reversible promoters can be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I (alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

For expression in a yeast cell, a suitable promoter is a constitutive promoter such as an ADH1 promoter, a PGK1 promoter, an ENO promoter, a PYK1 promoter and the like; or a regulatable promoter such as a GAL1 promoter, a GAL10 promoter, an ADH2 promoter, a PHOS promoter, a CUP1 promoter, a GALT promoter, a MET25 promoter, a MET3 promoter, a CYC1 promoter, a HIS3 promoter, an ADH1 promoter, a PGK promoter, a GAPDH promoter, an ADC1 promoter, a TRP1 promoter, a URA3 promoter, a LEU2 promoter, an ENO promoter, a TP1 promoter, and AOX1 (e.g., for use in Pichia). Selection of the appropriate vector and promoter is well within the level of ordinary skill in the art. Suitable promoters for use in prokaryotic host cells include, but are not limited to, a bacteriophage T7 RNA polymerase promoter; a trp promoter; a lac operon promoter; a hybrid promoter, e.g., a lac/tac hybrid promoter, a tac/trc hybrid promoter, a trp/lac promoter, a T7/lac promoter; a tre promoter; a tac promoter, and the like; an araBAD promoter; in vivo regulated promoters, such as an ssaG promoter or a related promoter (see, e.g., U.S. Patent Publication No. 20040131637), a pagC promoter (Pulkkinen and Miller, J. Bacteriol. (1991) 173 (1): 86-93; Alpuche-Aranda et al., Proc. Natl. Acad. Sci. USA (1992) 89 (21): 10079-83), a nirB promoter (Harborne et al. Mol. Micro. (1992) 6:2805-2813), and the like (see, e.g., Dunstan et al., Infect. Immun. (1999) 67:5133-5141; Mckelvie et al., Vaccine (2004) 22:3243-3255; and Chatfield et al., Biotechnol. (1992) 10:888-892); a sigma70 promoter, e.g., a consensus sigma70 promoter (see, e.g., GenBank Accession Nos. AX798980, AX798961, and AX798183); a stationary phase promoter, e.g., a dps promoter, an spv promoter, and the like; a promoter derived from the pathogenicity island SPI-2 (see, e.g., WO96/17951); an actA promoter (see, e.g., Shetron-Rama et al., Infect. Immun. (2002) 70:1087-1096); an rpsM promoter (see, e.g., Valdivia and Falkow Mol. Microbiol. (1996). 22:367); a tet promoter (see, e.g., Hillen, W. and Wissmann, A. (1989) In Saenger, W. and Heinemann, U. (eds), Topics in Molecular and Structural Biology, Protein-Nucleic Acid Interaction. Macmillan, London, UK, Vol. 10, pp. 143-162); an SP6 promoter (see, e.g., Melton et al., Nucl. Acids Res. (1984) 12:7035); and the like. Suitable strong promoters for use in prokaryotes such as Escherichia coli include, but are not limited to Trc, Tac, T5, T7, and PLambda. Non-limiting examples of operators for use in bacterial host cells include a lactose promoter operator (LacI repressor protein changes conformation when contacted with lactose, thereby preventing the Lad repressor protein from binding to the operator), a tryptophan promoter operator (when complexed with tryptophan, TrpR repressor protein has a conformation that binds the operator; in the absence of tryptophan, the TrpR repressor protein has a conformation that does not bind to the operator), and a tac promoter operator (see, e.g., deBoer et al., Proc. Natl. Acad. Sci. U.S.A. (1983) 80:21-25).

Other examples of suitable promoters include the immediate early cytomegalovirus (CMV) promoter sequence. This promoter sequence is a strong constitutive promoter sequence capable of driving high levels of expression of any polynucleotide sequence operatively linked thereto. Other constitutive promoter sequences can also be used, including, but not limited to a simian virus 40 (SV40) early promoter, a mouse mammary tumor virus (MMTV) or human immunodeficiency virus (HIV) long terminal repeat (LTR) promoter, a MoMuL V promoter, an avian leukemia virus promoter, an Epstein-Barr virus immediate early promoter, a Rous sarcoma virus promoter, the EF-1 alpha promoter, as well as human gene promoters such as, but not limited to, an actin promoter, a myosin promoter, a hemoglobin promoter, and a creatine kinase promoter. Further, the present disclosure should not be limited to the use of constitutive promoters. Inducible promoters are also contemplated as part of the present disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

In some embodiments, the locus or construct or transgene containing the suitable promoter is irreversibly switched through the induction of an inducible system. Suitable systems for induction of an irreversible switch are well known in the art, e.g., induction of an irreversible switch can make use of a Cre-lox-mediated recombination (see, e.g., Fuhrmann-Benzakein, et al., Proc. Natl. Acad. Sci. USA (2000) 28: e99, the present disclosure of which is incorporated herein by reference). Any suitable combination of recombinase, endonuclease, ligase, recombination sites, etc. known to the art can be used in generating an irreversibly switchable promoter. Methods, mechanisms, and requirements for performing site-specific recombination, described elsewhere herein, find use in generating irreversibly switched promoters and are well known in the art, see, e.g., Grindley et al. Annual Review of Biochemistry (2006) 567-605; and Tropp, Molecular Biology (2012) (Jones & Bartlett Publishers, Sudbury, Mass.), the present disclosures of which are incorporated herein by reference.

A nucleic acid of the present disclosure can be present within an expression vector and/or a cloning vector. An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host can be present. Suitable expression vectors include, but are not limited to, viral vectors (e.g., viral vectors based on vaccinia virus; poliovirus; adenovirus (see, e.g., Li et al., Invest. Opthalmol. Vis. Sci. (1994) 35:2543-2549; Borras et al., Gene Ther. (1999) 6:515-524; Li and Davidson, Proc. Natl. Acad. Sci. USA (1995) 92:7700-7704; Sakamoto et al., H. Gene Ther. (1999) 5:1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9:81-86, Flannery et al., Proc. Natl. Acad. Sci. USA (1997) 94:6916-6921; Bennett et al., Invest. Opthalmol. Vis. Sci. (1997) 38:2857-2863; Jomary et al., Gene Ther. (1997) 4:683 690, Rolling et al., Hum. Gene Ther. (1999) 10:641-648; Ali et al., Hum. Mol. Genet. (1996) 5:591-594; Srivastava in WO 93/09239, Samulski et al., J. Vir. (1989) 63:3822-3828; Mendelson et al., Virol. (1988) 166: 154-165; and Flotte et al., Proc. Natl. Acad. Sci. USA (1993) 90:10613-10617); SV40; herpes simplex virus; human immunodeficiency virus (see, e.g., Miyoshi et al., Proc. Natl. Acad. Sci. USA (1997) 94:10319-23; Takahashi et al., J. Virol. (1999) 73:7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

In some embodiments, an expression vector (e.g., a lentiviral vector) can be used to introduce the nucleic acid into a host cell. Accordingly, an expression vector (e.g., a lentiviral vector) of the present disclosure can comprise a nucleic acid encoding a polypeptide. In some embodiments, the expression vector (e.g., lentiviral vector) will comprise additional elements that will aid in the functional expression of the polypeptide encoded therein. In some embodiments, an expression vector comprising a nucleic acid encoding for a polypeptide further comprises a mammalian promoter. In one embodiment, the vector further comprises an elongation-factor-1-alpha promoter (EF-1α promoter). Use of an EF-1α promoter can increase the efficiency in expression of downstream transgenes. Physiologic promoters (e.g., an EF-1α promoter) can be less likely to induce integration mediated genotoxicity and can abrogate the ability of the retroviral vector to transform stem cells. Other physiological promoters suitable for use in a vector (e.g., lentiviral vector) are known to those of skill in the art and can be incorporated into a vector of the present disclosure. In some embodiments, the vector (e.g., lentiviral vector) further comprises a non-requisite cis acting sequence that can improve titers and gene expression. One non-limiting example of a non-requisite cis acting sequence is the central polypurine tract and central termination sequence (cPPT/CTS) which is important for efficient reverse transcription and nuclear import. Other non-requisite cis acting sequences are known to those of skill in the art and can be incorporated into a vector (e.g., lentiviral vector) of the present disclosure. In some embodiments, the vector further comprises a posttranscriptional regulatory element. Posttranscriptional regulatory elements can improve RNA translation, improve transgene expression and stabilize RNA transcripts. One example of a posttranscriptional regulatory element is the woodchuck hepatitis virus posttranscriptional regulatory element (WPRE). Accordingly, in some embodiments a vector for the present disclosure further comprises a WPRE sequence. Various posttranscriptional regulator elements are known to those of skill in the art and can be incorporated into a vector (e.g., lentiviral vector) of the present disclosure. A vector of the present disclosure can further comprise additional elements such as a rev response element (RRE) for RNA transport, packaging sequences, and 5′ and 3′ long terminal repeats (LTRs). The term “long terminal repeat” or “LTR” refers to domains of base pairs located at the ends of retroviral DNAs which comprise U3, R and U5 regions. LTRs generally provide functions required for the expression of retroviral genes (e.g., promotion, initiation and polyadenylation of gene transcripts) and to viral replication. In one embodiment, a vector (e.g., lentiviral vector) of the present disclosure includes a 3′ U3 deleted LTR. Accordingly, a vector (e.g., lentiviral vector) of the present disclosure can comprise any combination of the elements described herein to enhance the efficiency of functional expression of transgenes. For example, a vector (e.g., lentiviral vector) of the present disclosure can comprise a WPRE sequence, cPPT sequence, RRE sequence, 5′LTR, 3′ U3 deleted LTR′ in addition to a nucleic acid encoding for a CAR.

Vectors of the present disclosure can be self-inactivating vectors. As used herein, the term “self-inactivating vector” refers to vectors in which the 3′ LTR enhancer promoter region (U3 region) has been modified (e.g., by deletion or substitution). A self-inactivating vector can prevent viral transcription beyond the first round of viral replication. Consequently, a self-inactivating vector can be capable of infecting and then integrating into a host genome (e.g., a mammalian genome) only once, and cannot be passed further. Accordingly, self-inactivating vectors can greatly reduce the risk of creating a replication-competent virus.

In some embodiments, a nucleic acid of the present disclosure can be RNA, e.g., in vitro synthesized RNA. Methods for in vitro synthesis of RNA are known to those of skill in the art; any known method can be used to synthesize RNA comprising a sequence encoding a polypeptide of the present disclosure. Methods for introducing RNA into a host cell are known in the art. See, e.g., Zhao et al. Cancer Res. (2010) 15:9053. Introducing RNA comprising a nucleotide sequence encoding a polypeptide of the present disclosure into a host cell can be carried out in vitro, ex vivo or in vivo. For example, a host cell (e.g., an NK cell, a cytotoxic T lymphocyte, etc.) can be electroporated in vitro or ex vivo with RNA comprising a nucleotide sequence encoding a polypeptide of the present disclosure.

In order to assess the expression of a polypeptide or portions thereof, the expression vector to be introduced into a cell can also contain either a selectable marker gene or a reporter gene, or both, to facilitate identification and selection of expressing cells from the population of cells sought to be transfected or infected through viral vectors. In some embodiments, the selectable marker can be carried on a separate piece of DNA and used in a co-transfection procedure. Both selectable markers and reporter genes can be flanked with appropriate regulatory sequences to enable expression in the host cells. Useful selectable markers include, without limitation, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assessed at a suitable time after the DNA has been introduced into the recipient cells. Suitable reporter genes can include, without limitation, genes encoding luciferase, beta-galactosidase, chloramphenicol acetyl transferase, secreted alkaline phosphatase, or the green fluorescent protein gene (e.g., Ui-Tei et al., 2000 FEBS Letters 479:79-82).

In some embodiments, a nucleic acid of the present disclosure is provided for the production of a polypeptide as described herein, e.g., in a host cell. In some embodiments, a nucleic acid of the present disclosure provides for amplification of the polypeptide-encoding nucleic acid.

### Immunoconjugates

The present disclosure also provides immunoconjugates comprising any of the anti-cPD-1 antibodies or antigen-binding fragments, or the scFv disclosed herein. In some embodiments, the immunoconjugate comprises an antibody or antigen-binding fragments, or the scFv disclosed linked to an agent. In some embodiments, the immunoconjugate comprises a bispecific molecule disclosed herein linked to an agent. The agent can be a therapeutic agent. The agent can be a diagnostic agent.

For diagnostic agents, appropriate agents can include detectable labels. Non-limiting examples of detectable labels include radioisotopes, for whole body imaging, and radioisotopes, enzymes, fluorescent labels and other suitable antibody tags for sample testing. The detectable labels that can be linked to any anti-cPD-1 antibody described herein can be any of the various types used currently in the field of in vitro diagnostics, including particulate labels including metal sols such as colloidal gold, isotopes such as ¹²⁵I or ^{99m}Tc presented for instance with a peptidic chelating agent of the N2S2, N3S or N4 type, chromophores including fluorescent markers, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro {1,2-dioxetane-3,2′-(5′-chloro) tricyclo {3.3.1.1 3,7}decan}-4-yl) phenyl phosphate (CSPD), as well as CDP and CDP-STAR® or other luminescent substrates well-known to those in the art, for example the chelates of suitable lanthanides such as Terbium (III) and Europium (III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

In some embodiments, conjugation methods result in linkages which are substantially (or nearly) non-immunogenic, e.g., peptide—(i.e., amide-), sulfide-, (sterically hindered), disulfide-, hydrazone-, and ether linkages. These linkages are nearly non-immunogenic and show reasonable stability within serum; see, e.g., Senter, P. D., Curr. Opin. Chem. Biol. 13 (2009) 235-244; WO 2009/059278; WO 95/17886).

Depending on the biochemical nature of the agent and the anti-cPD-1 antibody or the scFv, different conjugation strategies can be employed. In case the agent is naturally-occurring or recombinant of between 50 to 500 amino acids, there are standard procedures in text books describing the chemistry for synthesis of protein conjugates, which can be easily followed by the skilled artisan (see, e.g., Hackenberger, C. P. R., and Schwarzer, D., Angew. Chem. Int. Ed. Engl. 47 (2008) 10030-10074). In some embodiments the reaction of a maleinimido agent with a cysteine residue within the antibody or the agent is used. This is an especially suited coupling chemistry in case e.g., a Fab or Fab′-fragment of an antibody is used. Alternatively, in some embodiments, coupling to the C-terminal end of the antibody or agent is performed. C-terminal modification of a protein, e.g., of a Fab-fragment, can be performed as described (Sunbul, M. and Yin, J., Org. Biomol. Chem. 7 (2009) 3361-3371).

In general, site specific reaction and covalent coupling is based on transforming a natural amino acid into an amino acid with a reactivity which is orthogonal to the reactivity of the other functional groups present. For example, a specific cysteine within a rare sequence context can be enzymatically converted in an aldehyde (see Frese, M. A., and Dierks, T., ChemBioChem. (2009) 425-427). It is also possible to obtain a desired amino acid modification by utilizing the specific enzymatic reactivity of certain enzymes with a natural amino acid in a given sequence context (see, e.g., Taki, M. et al., Prot. Eng. Des. Sel. 17 (2004) 119-126; Gautier, A. et al. Chem. Biol. 15 (2008) 128-136; and Protease-catalyzed formation of C—N bonds is used by Bordusa, F., Highlights in Bioorganic Chemistry (2004) 389-403). Site specific reaction and covalent coupling can also be achieved by the selective reaction of terminal amino acids with appropriate modifying reagents.

The reactivity of an N-terminal cysteine with benzonitriles (see Ren, H. et al., Angew. Chem. Int. Ed. Engl. 48 (2009) 9658-9662) can be used to achieve a site-specific covalent coupling.

Native chemical ligation can also rely on C-terminal cysteine residues (Taylor, E. Vogel; Imperiali, B, Nucleic Acids and Molecular Biology (2009), 22 (Protein Engineering), 65-96). U.S. Pat. No. 6,437,095 B1 describes a conjugation method which is based on the faster reaction of a cysteine within a stretch of negatively charged amino acids with a cysteine located in a stretch of positively charged amino acids.

The agent can also be a synthetic peptide or peptide mimic. In case a polypeptide is chemically synthesized, amino acids with orthogonal chemical reactivity can be incorporated during such synthesis (see e.g, de Graaf, A. J. et al., Bioconjug. Chem. 20 (2009) 1281-1295). Since a great variety of orthogonal functional groups is at stake and can be introduced into a synthetic peptide, conjugation of such peptide to a linker is standard chemistry.

In order to obtain a mono-labeled polypeptide, the conjugate with 1:1 stoichiometry can be separated by chromatography from other conjugation side-products. This procedure can be facilitated by using a dye labeled binding pair member and a charged linker. By using this kind of labeled and highly negatively charged binding pair member, mono conjugated polypeptides are easily separated from non-labeled polypeptides and polypeptides which carry more than one linker, since the difference in charge and molecular weight can be used for separation. The fluorescent dye can be useful for purifying the complex from un-bound components, like a labeled monovalent binder.

In some embodiments, the agent attached to an anti-cPD-1 antibody is selected from the group consisting of a binding moiety, a labeling moiety, and a biologically active moiety.

Anti-cPD-1 antibodies described herein can also be conjugated to a therapeutic agent to form an immunoconjugate such as an antibody-drug conjugate (ADC). Suitable therapeutic agents include antimetabolites, alkylating agents, DNA minor groove binders, DNA intercalators, DNA crosslinkers, histone deacetylase inhibitors, nuclear export inhibitors, proteasome inhibitors, topoisomerase I or II inhibitors, heat shock protein inhibitors, tyrosine kinase inhibitors, antibiotics, and anti-mitotic agents. In the ADC, the antibody and therapeutic agent preferably are conjugated via a linker cleavable such as a peptidyl, disulfide, or hydrazone linker. In some embodiments, the linker is a peptidyl linker such as Val-Cit, Ala-Val, Val-Ala-Val, Lys-Lys, Pro-Val-Gly-Val-Val (SEQ ID NO: 78), Ala-Asn-Val, Val-Leu-Lys, Ala-Ala-Asn, Cit-Cit, Val-Lys, Lys, Cit, Ser, or Glu. The ADCs can be prepared as described in U.S. Pat. Nos. 7,087,600; 6,989,452; and 7,129,261; PCT Publications WO 02/096910; WO 07/038658; WO 07/051081; WO 07/059404; WO 08/083312; and WO 08/103693; U.S. Patent Publications 20060024317; 20060004081; and 20060247295.

Anti-cPD-1 antibodies or scFv described herein, can also be used for detecting canine PD-1, e.g., cPD-1 in tissues or tissue samples. The antibodies can be used, e.g., in an ELISA assay or in flow cytometry. In some embodiments, an anti-cPD-1 antibody is contacted with cells, e.g., cells in a tissue, for a time appropriate for specific binding to occur, and then a reagent, e.g., an antibody that detects the anti-cPD-1 antibody, is added. Exemplary assays are provided in the Examples. The anti-cPD-1 antibody can be a fully canine antibody. The anti-cPD-1 antibody can be a caninized or chimeric antibody. Exemplary methods for detecting cPD-1 in a sample (cell or tissue sample) comprise (i) contacting a sample with an anti-cPD-1 antibody, for a time sufficient for allowing specific binding of the anti-cPD-1 antibody to cPD-1 in the sample, and (2) contacting the sample with a detection reagent, e.g., an antibody, that specifically binds to the anti-cPD-1 antibody, such as to the Fc region of the anti-cPD-1 antibody, to thereby detect cPD-1 bound by the anti-cPD-1 antibody. Wash steps can be included after the incubation with the antibody and/or detection reagent. Anti-cPD-1 antibodies for use in these methods do not have to be linked to a label or detection agents, as a separate detection agent can be used.

### Bispecific Molecules

Anti-cPD-1 antibodies, antigen-binding fragments thereof, or the scFv described herein can be used for forming bispecific molecules. An anti-cPD-1 antibody, or antigen-binding portions thereof, can be derivatized or linked to another functional molecule, e.g., another peptide or protein (e.g., another antibody or ligand for a receptor) to generate a bispecific molecule that binds to at least two different binding sites or target molecules.

For example, an anti-cPD-1 antibody can be linked to an antibody or scFv that binds specifically to any protein that can be used as potential targets for combination treatments. The antibody described herein can in fact be derived or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule described herein, an antibody described herein can be functionally linked (e.g., by chemical coupling, genetic fusion, noncovalent association or otherwise) to one or more other binding molecules, such as another antibody, antibody fragment, peptide or binding mimetic, such that a bispecific molecule results.

Provided herein are bispecific molecules comprising at least one first binding specificity for canine PD-1 and a second binding specificity for a second target epitope. In some embodiments described herein in which the bispecific molecule is multi-specific, the molecule can further include a third binding specificity.

In some embodiments, the bispecific molecules described herein comprise as a binding specificity at least one antibody, or an antibody fragment thereof, including, e.g., an Fab, Fab′, F(ab′)2, Fv, or a single chain Fv (scFv). The antibody can also be a light chain or heavy chain dimer, or any minimal fragment thereof such as a Fv or a single chain construct.

In some embodiments, the canine monoclonal antibodies are used in the bispecific molecule. In other embodiments, other antibodies can be employed in the bispecific molecules described herein are murine, chimeric and caninized monoclonal antibodies.

The bispecific molecules described herein can be prepared by conjugating the constituent binding specificities using methods known in the art. For example, each binding specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis (2-nitrobenzoic acid) (DTNB), o-phenylenedimaleimide (oPDM), N-succinimidyl-3-(2-pyridyldithio) propionate (SPDP), and sulfosuccinimidyl 4-(N-maleimidomethyl) cyclohaxane-1-carboxylate (sulfo-SMCC) (see, e.g., Karpovsky et al. (1984) J. Exp. Med. 160:1686; Fiu, M A et al. (1985) Proc. Natl. Acad. Sci. USA 82:8648). Other methods include those described in Paulus (1985) Behring Ins. Mitt. No. 78, 118-132; Brennan et al. (1985) Science 229:81-83), and Glennie et al. (1987) J. Immunol. 139:2367-2375). Some conjugating agents are SATA and sulfo-SMCC, both available from Pierce Chemical Co. (Rockford, IL).

When the binding specificities are antibodies, they can be conjugated via sulfhydryl bonding of the C-terminus hinge regions of the two heavy chains. In some embodiments, the hinge region is modified to contain an odd number of sulfhydryl residues, preferably one, prior to conjugation.

Alternatively, both binding specificities can be encoded in the same vector and expressed and assembled in the same host cell. This method is particularly useful where the bispecific molecule is a mAh x mAh, mAh x Fab, mAh x (scFv)2, Fab x F(ab′)2 or ligand x Fab fusion protein. A bispecific antibody can comprise an antibody comprising an scFv at the C-terminus of each heavy chain. A bispecific molecule described herein can be a single chain molecule comprising one single chain antibody and a binding determinant, or a single chain bispecific molecule comprising two binding determinants. Bispecific molecules can comprise at least two single chain molecules. Methods for preparing bispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the bispecific molecules to their specific targets can be confirmed using art-recognized methods, such as enzyme-linked immunosorbent assay (ELISA), radioimmunoassay (RIA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

### Methods of Treatment

In some aspects, the present disclosure provides a method for inhibiting canine programmed death protein 1 (cPD-1) signaling pathway in a subject in need thereof comprising administering to the subject the antibodies, binding polypeptides, and scFvs described herein, or a bispecific molecule, an immunoconjugate, or a composition comprising any one of these. In some cases, the subject has cancer, sepsis or septic shock, or a chronic infection (e.g., viral infections). The composition can include a pharmaceutical composition and further include a pharmaceutically acceptable carrier. A therapeutically effective amount of the pharmaceutical composition can be administered to the subject (e.g., a dog or canine).

In some aspects, the present disclosure provides a method for increasing T cell proliferation and reducing T cell exhaustion in a subject in need thereof comprising administering to the subject the antibodies, binding polypeptides, and scFvs described herein, or a bispecific molecule, an immunoconjugate, or a composition comprising any one of these. In some cases, the subject has cancer, sepsis or septic shock, or a chronic infection (e.g., viral infections). The composition can include a pharmaceutical composition and further include a pharmaceutically acceptable carrier. A therapeutically effective amount of the pharmaceutical composition can be administered to the subject (e.g., a dog or canine). In some cases, the T cell proliferation in the canine subject is increased by at least 10%, 20%, 30%, 40%, 50%, 60%, 70%, 80%, 90%, 100%, 150%, 200%, 300%, 400%, 500% relative to a comparable canine subject that was not given the antibodies, binding polypeptides, and scFvs described herein, or a bispecific molecule, an immunoconjugate, or a composition comprising any one of these described herein. In some cases, the T cell exhaustion in the canine subject is reduced by at least 10%, 20%, 30%, 40%, 50%, 60%, 70%, 80%, 90%, 95%, relative to a comparable canine subject that was not given the antibodies, binding polypeptides, and scFvs described herein, or a bispecific molecule, an immunoconjugate, or a composition comprising any one of these described herein. In some embodiments, the T cell is an effector T cell. In some embodiments, the T cell is a CD5+ T cell.

In some aspects, the present disclosure provides a method for increasing IFNγ production in a subject in need thereof comprising administering to the subject the antibodies, binding polypeptides, and scFvs described herein, or a bispecific molecule, an immunoconjugate, or a composition comprising any one of these. In some cases, the subject has cancer, sepsis or septic shock, or a chronic infection (e.g., viral infections). The composition can include a pharmaceutical composition and further include a pharmaceutically acceptable carrier. A therapeutically effective amount of the pharmaceutical composition can be administered to the subject (e.g., a dog or canine). In some cases, the IFNγ production in the canine subject is increased by at least 10%, 20%, 30%, 40%, 50%, 60%, 70%, 80%, 90%, 100%, 150%, 200%, 300%, 400%, 500% relative to a comparable canine subject that was not given the antibodies, binding polypeptides, and scFvs described herein, or a bispecific molecule, an immunoconjugate, or a composition comprising any one of these described herein. In some embodiments, the T cell is a CD5+ T cell.

In one aspect, the present disclosure provides a method for treating a disease or condition in a subject in need thereof comprising administering to the subject the antibodies, binding polypeptides, and scFvs described herein, or a bispecific molecule, an immunoconjugate, or a composition comprising any one of these. The disease or condition comprises cancer, sepsis or septic shock, or a chronic infection (e.g., viral infections). The composition can include a pharmaceutical composition and further include a pharmaceutically acceptable carrier. A therapeutically effective amount of the pharmaceutical composition can be administered to the subject (e.g., a dog or canine).

In certain embodiments, described herein is the antibodies, binding polypeptides, and scFvs described herein, or a bispecific molecule, an immunoconjugate, or a composition comprising any one of these, for use as a medicament.

In certain embodiments, described herein is the antibodies, binding polypeptides, and scFvs described herein, or a bispecific molecule, an immunoconjugate, or a composition comprising any one of these, for use as a medicament for the treatment of cancer, sepsis or septic shock, or chronic infection.

In certain embodiments, described herein is use of the antibodies, binding polypeptides, and scFvs described herein, or a bispecific molecule, an immunoconjugate, or a composition comprising any one of these, for the manufacture of a medicament.

In certain embodiments, described herein is use of the antibodies, binding polypeptides, and scFvs described herein, or a bispecific molecule, an immunoconjugate, or a composition comprising any one of these, for the manufacture of a medicament for the treatment of cancer, sepsis or septic shock, or chronic infection (e.g., viral infections).

Treatment refers to a method that seeks to improve or ameliorate the condition being treated. With respect to cancer, treatment includes, but is not limited to, reduction of tumor volume, reduction in growth of tumor volume, increase in progression-free survival, or overall life expectancy. In certain embodiments, treatment will affect remission of a cancer being treated. In certain embodiments, treatment encompasses use as a prophylactic or maintenance dose intended to prevent reoccurrence or progression of a previously treated cancer or tumor. It is understood by those of skill in the art that not all individuals will respond equally or at all to a treatment that is administered, nevertheless these individuals are considered to be treated.

The method comprises administering to the subject an isolated binding polypeptide comprising a heavy chain variable region comprising an amino acid sequence at least 80%, 85%, 90%, 95%, 96%, 97%, 98%, 99%, or 100% identical to SEQ ID NOs: 7, 9, 33, or 49 and a light chain variable region comprising an amino acid sequence at least 80%, 85%, 90%, 95%, 96%, 97%, 98%, 99%, or 100% identical to SEQ ID NOs: 11, 35, or 51.

In certain embodiments, the cancer is associated with programmed death protein 1 (PD-1) signaling pathway. In certain embodiments, the PD-1 is expressed on an immune cell of the subject. In certain embodiments, the immune cell is a T lymphocyte. In certain embodiments, the immune cell is a tumor-infiltrating lymphocyte. In certain embodiments, a ligand of PD-1, such as PD-L1 or PD-L2 is expressed on a cancer cell of the subject. In certain embodiments, canine programmed death-ligand 1 is over-expressed on a cancer cell of the subject.

In certain embodiments, the binding polypeptide specifically binds to programmed death protein 1 (PD-1). In certain embodiments, the binding polypeptide comprises an antibody or an antigen-binding fragment thereof. In certain embodiments, the antigen-binding fragment is selected from the group consisting of a Fab, a single-chain variable fragment (scFv), a single-domain antibody, sc(Fv)2, dsFv, Fab, Fab′, (Fab′)2 and a diabody. In certain embodiments, the antibody is a full-length antibody. In certain embodiments, the antibody or antigen-binding fragment is a canine antibody or an antigen-binding fragment thereof.

In certain embodiments, the method further comprises administering one or more additional therapeutics or interventions. There is no limitation on such additional therapeutics or interventions, which can include any therapeutic agents or small molecule drugs that is helpful for treating the subject in need thereof. In some embodiments, the additional therapeutics or interventions are administered with the antibody or antigen-binding fragments thereof or the scFv described herein, or the bispecific molecule or the immunoconjugates comprising these, as a combination therapy. Non-limiting examples of additional therapeutics or interventions include chemotherapy (e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine), radiation therapy, immunotherapy, and other targeted therapy.

In certain embodiments, the cancer or tumor is a solid cancer or tumor. In certain embodiments, the cancer or tumor is a blood cancer or tumor. In certain embodiments, the cancer or tumor comprises breast, heart, lung, small intestine, colon, spleen, kidney, bladder, head, neck, ovarian, prostate, brain, pancreatic, skin, bone, bone marrow, blood, thymus, uterine, testicular, and liver tumors. In certain embodiments, tumors which can be treated with the antibodies of the present disclosure comprise adenoma, adenocarcinoma, angiosarcoma, astrocytoma, epithelial carcinoma, germinoma, glioblastoma, glioma, hemangioendothelioma, hemangiosarcoma, hematoma, hepatoblastoma, leukemia, lymphoma, medulloblastoma, melanoma, neuroblastoma, osteosarcoma, retinoblastoma, rhabdomyosarcoma, sarcoma and/or teratoma. In certain embodiments, the tumor/cancer is selected from the group of acral lentiginous melanoma, actinic keratosis, adenocarcinoma, adenoid cystic carcinoma, adenomas, adenosarcoma, adenosquamous carcinoma, astrocytic tumors, Bartholin gland carcinoma, basal cell carcinoma, bronchial gland carcinoma, capillary carcinoid, carcinoma, carcinosarcoma, cholangiocarcinoma, chondrosarcoma, cystadenoma, endodermal sinus tumor, endometrial hyperplasia, endometrial stromal sarcoma, endometrioid adenocarcinoma, ependymal sarcoma, Swing's sarcoma, focal nodular hyperplasia, gastronoma, germ line tumors, glioblastoma, glucagonoma, hemangioblastoma, hemangioendothelioma, hemangioma, hepatic adenoma, hepatic adenomatosis, hepatocellular carcinoma, insulinite, intraepithelial neoplasia, intraepithelial squamous cell neoplasia, invasive squamous cell carcinoma, large cell carcinoma, liposarcoma, lung carcinoma, lymphoblastic leukemia, lymphocytic leukemia, leiomyosarcoma, melanoma, malignant melanoma, malignant mesothelial tumor, nerve sheath tumor, medulloblastoma, medulloepithelioma, mesothelioma, mucoepidermoid carcinoma, myeloid leukemia, neuroblastoma, neuroepithelial adenocarcinoma, nodular melanoma, osteosarcoma, ovarian carcinoma, papillary serous adenocarcinoma, pituitary tumors, plasmacytoma, pseudosarcoma, prostate carcinoma, pulmonary blastoma, renal cell carcinoma, retinoblastoma, rhabdomyosarcoma, sarcoma, serous carcinoma, squamous cell carcinoma, small cell carcinoma, soft tissue carcinoma, somatostatin secreting tumor, squamous carcinoma, squamous cell carcinoma, undifferentiated carcinoma, uveal melanoma, verrucous carcinoma, vagina/vulva carcinoma, VIPpoma, and Wilm's tumor. In certain embodiments, the tumor/cancer to be treated with one or more antibodies of the present disclosure comprise brain cancer, head and neck cancer, colorectal carcinoma, acute myeloid leukemia, pre-B-cell acute lymphoblastic leukemia, bladder cancer, astrocytoma, preferably grade II, III or IV astrocytoma, glioblastoma, glioblastoma multiforme, small cell cancer, and non-small cell cancer, preferably non-small cell lung cancer, lung adenocarcinoma, metastatic melanoma, androgen-independent metastatic prostate cancer, androgen-dependent metastatic prostate cancer, prostate adenocarcinoma, and breast cancer, preferably breast ductal cancer, and/or breast carcinoma. In certain embodiments, the cancer treated with the antibodies of this disclosure comprises glioblastoma. In certain embodiments, the cancer treated with one or more antibodies of this disclosure comprises pancreatic cancer. In certain embodiments, the cancer treated with one or more antibodies of this disclosure comprises ovarian cancer. In certain embodiments, the cancer treated with one or more antibodies of this disclosure comprises lung cancer. In certain embodiments, the cancer treated with one or more antibodies of this disclosure comprises prostate cancer. In certain embodiments, the cancer treated with one or more antibodies of this disclosure comprises colon cancer. In certain embodiments, the cancer treated comprises glioblastoma, pancreatic cancer, ovarian cancer, colon cancer, prostate cancer, or lung cancer. In a certain embodiment, the cancer is refractory to other treatment. In a certain embodiment, the cancer treated is relapsed.

In some embodiments, the cancer comprises a melanoma (e.g., metastatic malignant melanoma), a prostate cancer (for example hormone refractory prostate adenocarcinoma), a head and neck cancer (for example, squamous cell carcinoma of the head and neck), a cervical cancer, a thyroid cancer, a glioblastoma, a glioma, leukemia, a lymphoma (for example, a B cell lymphoma), an adrenal gland cancer, an AIDS-associated cancer, an alveolar soft part sarcoma, an astrocytic tumor, bone cancer, a brain and spinal cord cancer, a metastatic brain tumor, a carotid body tumor, a chondrosarcoma, a chordoma, a chromophobe renal cell carcinoma, a clear cell carcinoma, cutaneous benign fibrous histiocytoma, a desmoplastic small round cell tumor, an ependymoma, a Ewing's tumor, an extra skeletal myxoid chondrosarcoma, a fibrogenesis imperfecta ossium, a fibrous dysplasia of the bone, a gallbladder or bile duct cancer, a gestational trophoblastic disease, a germ cell tumor, a hematological malignancy, hepatocellular carcinoma, an islet cell tumor, a Kaposi's sarcoma, a kidney cancer, a lipoma/benign lipomatous tumor, a liposarcoma/malignant lipomatous tumor, a medulloblastoma, a meningioma, a Merkel cell carcinoma, a multiple endocrine neoplasia, a multiple myeloma, a myelodysplasia syndrome, a neuroblastoma, a neuroendocrine tumor, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomyosarcoma, a sarcoma, a soft-tissue sarcoma, a squamous cell cancer, a stomach cancer, a synovial sarcoma, a testicular cancer, a thymic carcinoma, a thymoma, a thyroid metastatic cancer, a uterine cancer, or any combination thereof.

In some cases, the cancer comprises cervical cancer, lung cancers, liver cancers, ovarian cancers, cancers of the skin including melanoma and squamous cell carcinoma, colon cancer, bladder cancer, breast cancer, kidney cancer, esophageal cancer, stomach cancer, pancreatic cancers, head cancer, and neck cancer.

Compositions of the present disclosure can be administered in dosages and routes and at times to be determined in appropriate pre-clinical and clinical experimentation and trials. Compositions can be administered multiple times at dosages within these ranges. Administration of the compositions can be combined with other methods useful to treat the desired disease or condition as determined by those of skill in the art. In certain embodiments, the antibodies can be administered to a subject in need thereof by any route suitable for the administration of antibody-containing pharmaceutical compositions, such as, for example, subcutaneous, intraperitoneal, intravenous, intramuscular, intratumoral, or intracerebral, etc. In certain embodiments, the antibodies are administered intravenously. In certain embodiments, the antibodies are administered subcutaneously. In certain embodiments, the antibodies are administered intratumoral. In certain embodiments, the antibodies are administered on a suitable dosage schedule, for example, weekly, twice weekly, monthly, twice monthly, once every two weeks, once every three weeks, or once a month etc. In certain embodiments, the antibodies are administered once every three weeks. The antibodies can be administered in any therapeutically effective amount. In certain embodiments, therapeutically acceptable amount is between about 0.1 mg/kg and about 50 mg/kg. In certain embodiments, therapeutically acceptable amount is between about 1 mg/kg and about 40 mg/kg. In certain embodiments, therapeutically acceptable amount is between about 1 mg/kg and about 20 mg/kg. In certain embodiments, therapeutically acceptable amount is between about 1 mg/kg and about 10 mg/kg. In certain embodiments, therapeutically acceptable amount is between about 5 mg/kg and about 30 mg/kg. In certain embodiments, therapeutically acceptable amount is between about 5 mg/kg and about 20 mg/kg. Therapeutically effective amounts include amounts sufficient to ameliorate one or more symptoms associated with the disease or affliction to be treated.

In terms of the present disclosure, prophylactic, palliative, symptomatic and/or curative treatments may represent separate aspects of the disclosure. An anti-cPD-1 antibody or antigen-binding fragments thereof or scFv disclosed herein can be administered parenterally, such as intravenously, such as intramuscularly, such as subcutaneously. Alternatively, an antibody of the present disclosure can be administered via a non-parenteral route, such as orally or topically. An antibody of the invention can be administered prophylactically. An antibody of the present disclosure can be administered therapeutically (on demand).

Pharmaceutical Compositions, Kits, and Methods of making the compositions

Also provided are pharmaceutical composition comprising any one of the binding polypeptides, scFvs, antibodies, or the antigen-binding fragments disclosed herein. Among the compositions are pharmaceutical compositions and formulations for administration, such as for treatment of a disease or disorder. Also provided are therapeutic methods for administering the pharmaceutical compositions to subjects, e.g., canines.

The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular composition and/or by the method of administration. Accordingly, there are a variety of suitable formulations. For example, the pharmaceutical composition can contain preservatives. Suitable preservatives can include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for the particular indication, disease, or condition being treated with the composition, preferably those with activities complementary to the composition, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the composition in an amount effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the composition, by multiple bolus administrations of the composition, or by continuous infusion administration of the composition.

Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, and intraperitoneal administration. In some embodiments, the composition is administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which can in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the composition in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility can be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

In certain embodiments the anti-canine PD-1 antibodies of the current disclosure are included in a pharmaceutical composition comprising one or more pharmaceutically acceptable excipients, carriers, and diluents. Pharmaceutically acceptable excipients, carriers and diluents can be included to increase shelf-life, stability, or the administrability of the antibody. Such compounds include salts, pH buffers, detergents, anti-coagulants, and preservatives. In certain embodiments, the antibodies of the current disclosure are administered suspended in a sterile solution. In certain embodiments, the solution comprises about 0.9% NaCl. In certain embodiments, the solution comprises about 5.0% dextrose. In certain embodiments, the solution further comprises one or more of: buffers, for example, acetate, citrate, histidine, succinate, phosphate, bicarbonate and hydroxymethylaminomethane (Tris); surfactants, for example, polysorbate 80 (Tween 80), polysorbate 20 (Tween 20), and poloxamer 188; polyol/disaccharide/polysaccharides, for example, glucose, dextrose, mannose, mannitol, sorbitol, sucrose, trehalose, and dextran 40; amino acids, for example, glycine or arginine; antioxidants, for example, ascorbic acid, methionine; or chelating agents, for example, EDTA or EGTA.

In certain embodiments, the antibodies of the current disclosure can be shipped/stored lyophilized and reconstituted before administration. In certain embodiments, lyophilized antibody formulations comprise a bulking agent such as, mannitol, sorbitol, sucrose, trehalose, dextran 40, or combinations thereof. The lyophilized formulation can be contained in a vial comprised of glass or other suitable non-reactive material. The antibodies when formulated, whether reconstituted or not, can be buffered at a certain pH, generally less than 7.0. In certain embodiments, the pH can be between 4.5 and 7.0, 4.5 and 6.5, 4.5 and 6.0, 4.5 and 5.5, 4.5 and 5.0, or 5.0 and 6.0.

Also described herein are kits comprising one or more of the antibodies described herein in a suitable container and one or more additional components selected from: instructions for use; a diluent, an excipient, a carrier, and a device for administration.

In certain embodiments, described herein is a method of making a composition for treating cancer, sepsis or septic shock, or chronic infection (e.g., viral infections), comprising admixing one or more pharmaceutically acceptable excipients, carriers, or diluents and an antibody of the current disclosure. In certain embodiments, described herein is a method of preparing a cancer treatment for storage or shipping comprising lyophilizing one or more antibodies of the current disclosure.

While the present disclosure has been described with reference to the specific embodiments thereof, it should be understood by those skilled in the art that various changes can be made, and equivalents can be substituted without departing from the true spirit and scope of the present disclosure. It will be readily apparent to those skilled in the art that other suitable modifications and adaptations of the methods described herein can be made using suitable equivalents without departing from the scope of the embodiments disclosed herein. In addition, many modifications can be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### EXAMPLES

The present disclosure is further described in detail by reference to the following experimental examples. These examples are provided for purposes of illustration only and are not intended to be limiting unless otherwise specified. Thus, the present disclosure should in no way be construed as being limited to the following examples, but rather, should be construed to encompass any and all variations which become evident as a result of the teaching provided herein. While preferred embodiments of the present invention have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments of the present disclosure described herein can be employed in practicing the present disclosure.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Without further description, it is believed that one of ordinary skill in the art can, using the preceding description and the following illustrative examples, make and utilize the compounds of the present disclosure and practice the claimed methods. The following working examples, therefore, specifically point out some embodiments of the present disclosure.

### Example 1: Generation of Canine Anti-Canine PD-1 scFv

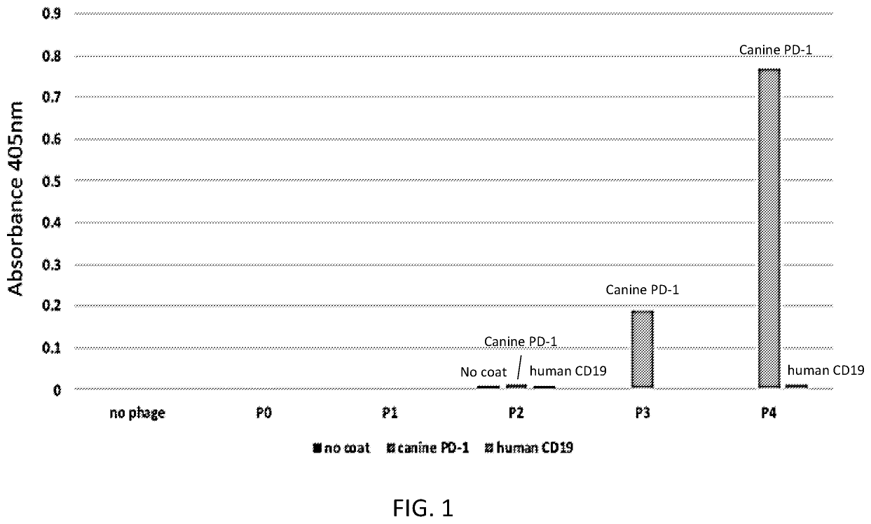

A canine IgM/IgG/N/K scFv phage display library containing an estimated 40 billion individual E. coli transformants was constructed from canine B cell mRNA and the pComb3X phagemid vector as described previously using oligonucleotide primers based on published canine immunoglobulin heavy and light chain germline genes. (Andris-Widhopf, J., Steinberger, P., Fuller, R., Rader, C., Barbas, C. F Generation of antibody libraries: PCR amplification and assembly of light- and heavy-chain coding sequences in Barbas, C. F., Burton, D. R., Scott, J. K, Silverman G. J. Phage Display: A Laboratory Manual, Cold Spring Harbor Laboratory Press (2001) and Lefranc, M. P., et al. IMGT, the international ImMunoGeneTics information system. Nucleic Acids Res 37, D1006-1012 (2009)). An aliquot of the library underwent 4 rounds of solid phase selection (“panning”) against canine PD-1. To verify that selected phage contained cPD-1-specific scFv phage particles, polyclonal scFv phage from each round of panning was evaluated by scFv phage ELISA using cPD-1 as the target antigen (FIG. 1). Substantial enrichment of phage for cPD-1 specific binders was identified in the third round of panning (P3) and increased in the fourth round of panning (P4).

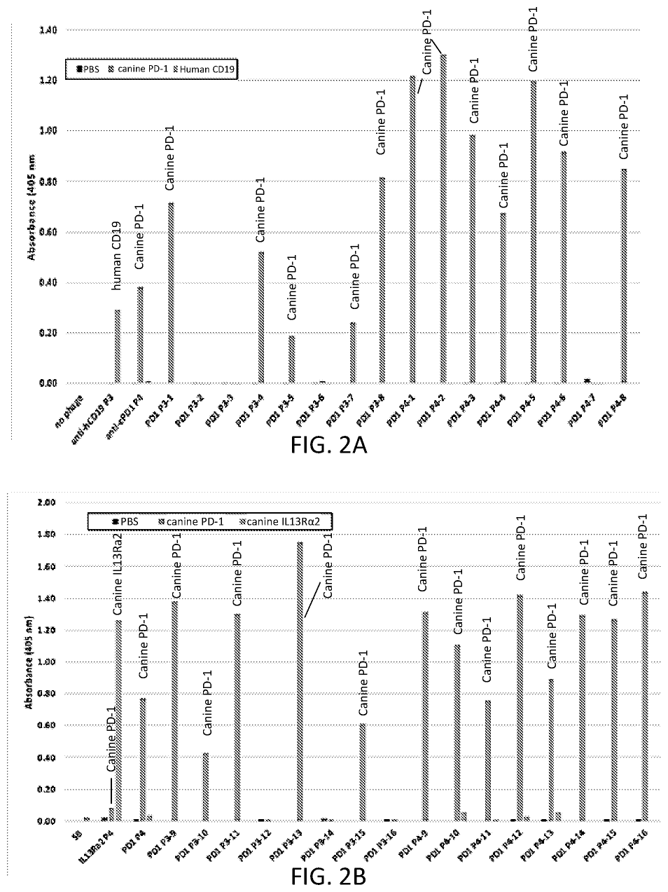

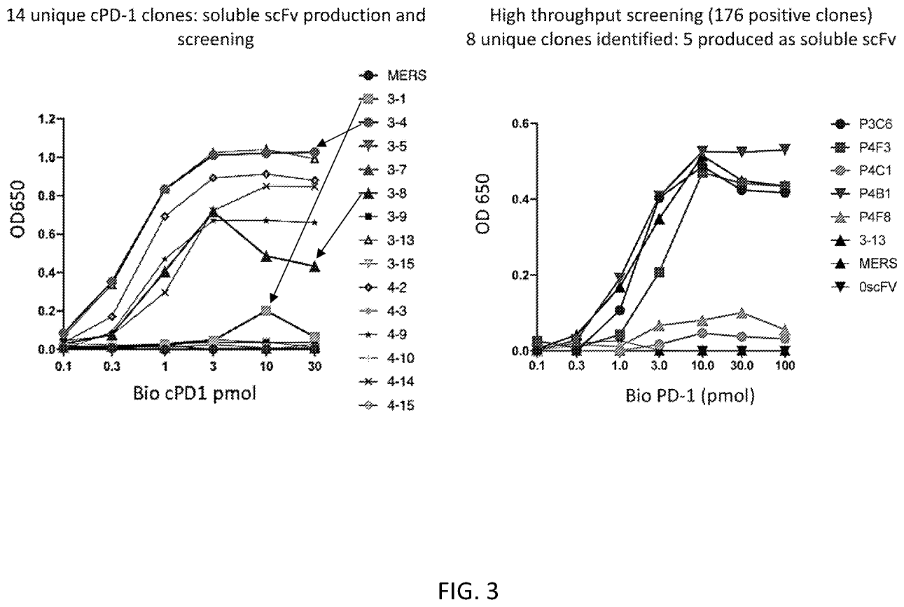

An initial survey of positive hit rate was assessed by examining 16 clones from panning round 3 and 16 clones from panning round 4 by testing their ability to bind to cPD-1 by phage ELISA. 10/16 clones from P3 and 15/16 clones from P4 bound to cPD-1 with a range of apparent affinities (FIGS. 2A-2B). 14 unique clones were identified by nucleotide sequencing from these 25 binders and were made as soluble scFv for further characterization (FIG. 3 left graph).

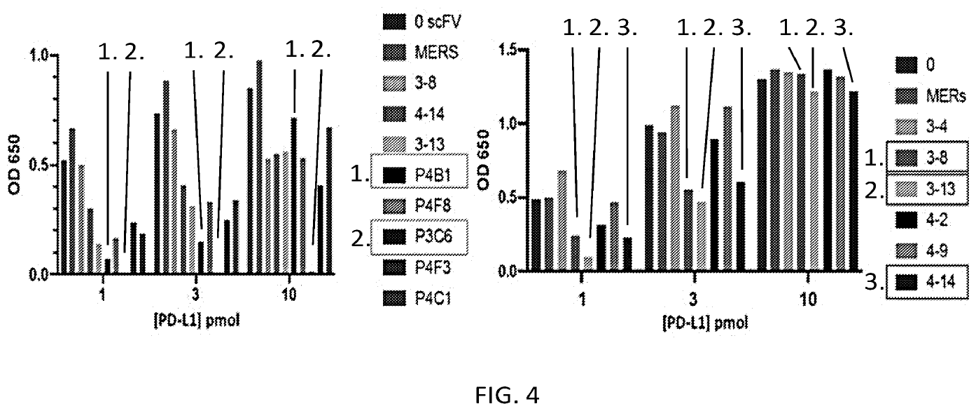

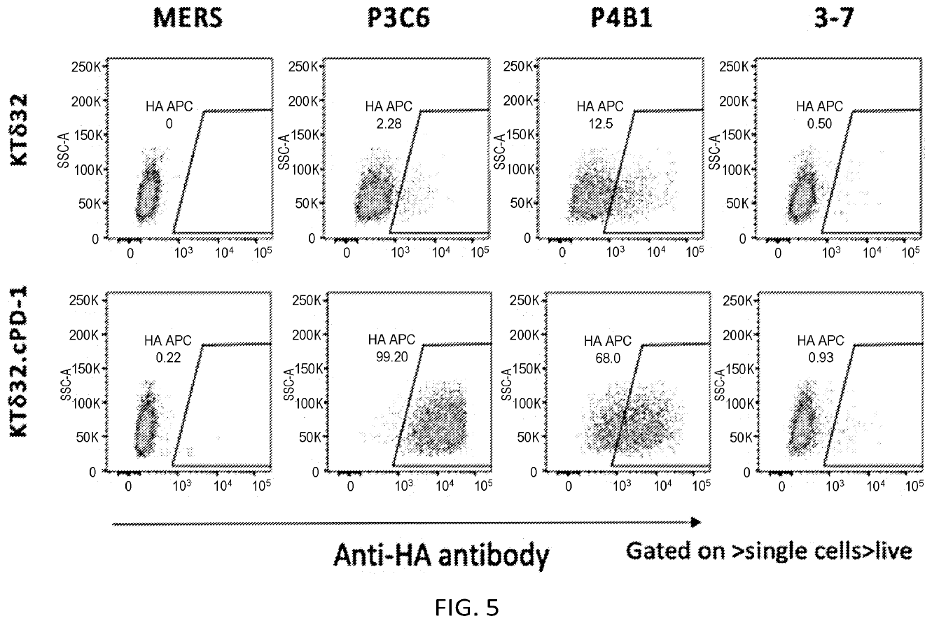

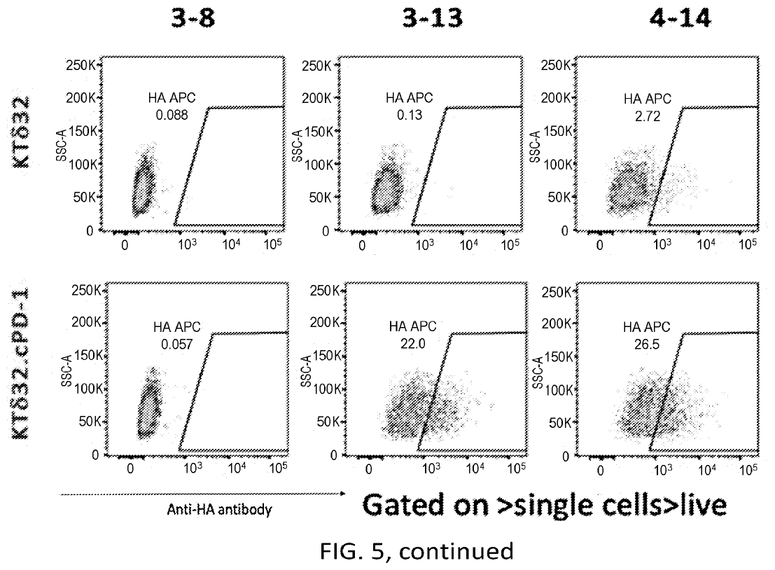

Following this initial survey to see positivity rate, a high-throughput screen of over several hundred additional clones was performed. 176 positive clones were isolated, of which a further 8 unique clones were identified. Five of these 8 additional clones were produced as soluble scFv for further characterization. Based on the results of soluble scFv binding to cPD-1 (FIG. 3), clones 3-4, 3-8, 3-13, 4-2, 4-9, 4-14, P4B1, P3C6 and P4F3 were selected for further analysis of their ability to inhibit the interaction of PD-1 with PD-L1 (FIG. 4). Five clones were detected that showed evidence of their ability to inhibit the interaction between PD-1 and PD-L1 (shown in the numbered boxes in FIG. 4). These clones were then tested for their ability to bind to membrane expressed cPD-1. The human erythroleukemic cell line K562 edited to remove CD32 (KTδ32) (to reduce non-specific binding), was engineered to express cPD-1 using a retroviral vector carrying a puromycin selection cassette (KTδ32.cPD-1). HA-tagged soluble scFvs selected via the inhibition assay (FIG. 4) were incubated with either KTδ32 or KTδ32.cPD-1 cells and binding was determined using an anti-HA antibody and flow cytometry (FIG. 5).

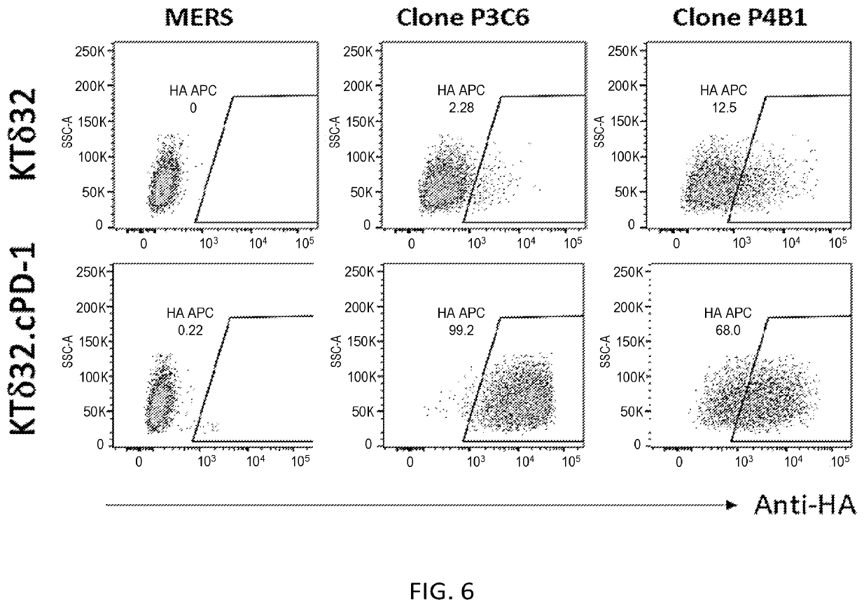

Clones P3C6 and P4B1 that inhibited PD-1: PD-L1 protein interactions and bound to cell surface expressed PD-1 were re-formatted as full length, fully canine IgG4 molecules and tested again for their ability to bind cell surface PD-1 by flow cytometry (FIG. 6).

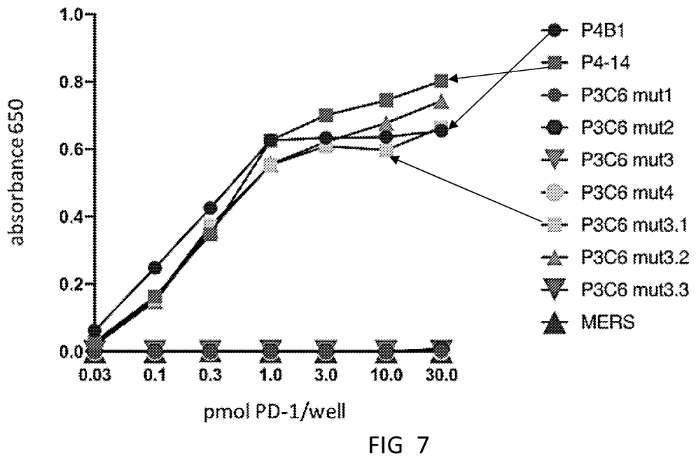

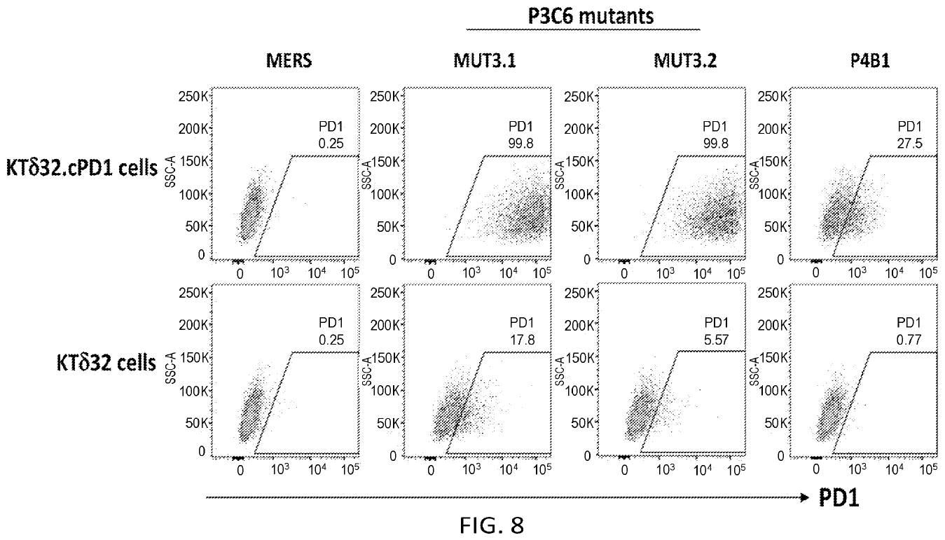

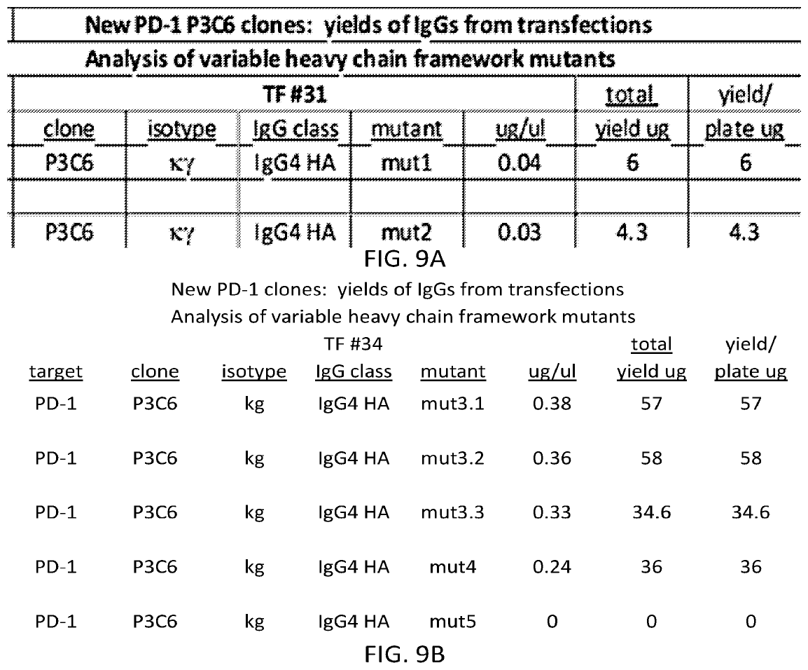

The yield of P3C6 achieved from transient transfection of 293T cells was low and chain swapping experiments confirmed that this was associated with the V_{H} chain of P3C6 (data not shown). Strategic amino acid replacements were made and the resultant mutant V_{H} chains were paired with the original P3C6 light chain and evaluated for production and cPD-1 binding capability (FIG. 7 and FIG. 8). Initial attempts to mutate P3C6 also led to low yields (FIG. 9A). After several attempts, the yields of two specific mutants, P3C6 mut 3.1 and mut 3.2, were significantly improved (FIG. 9B).

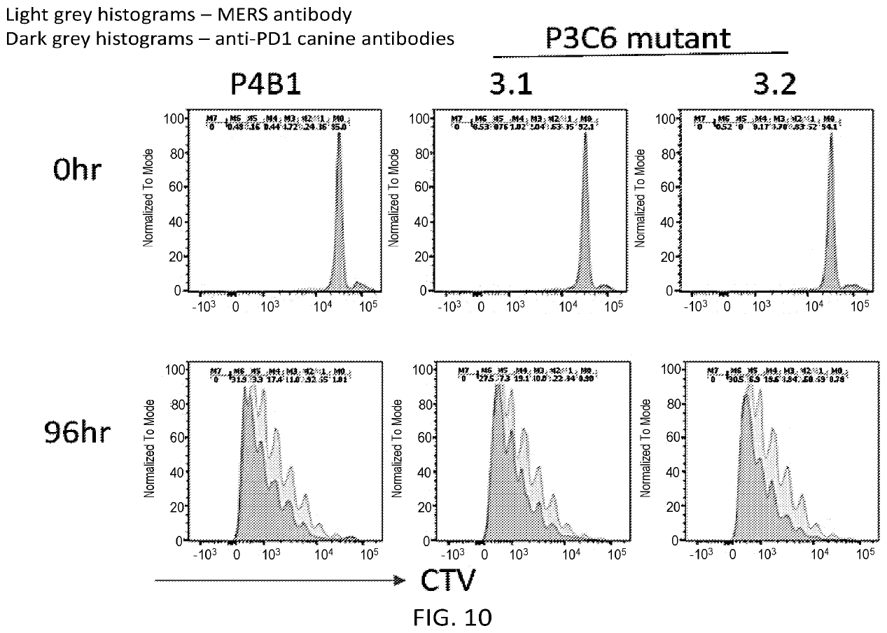

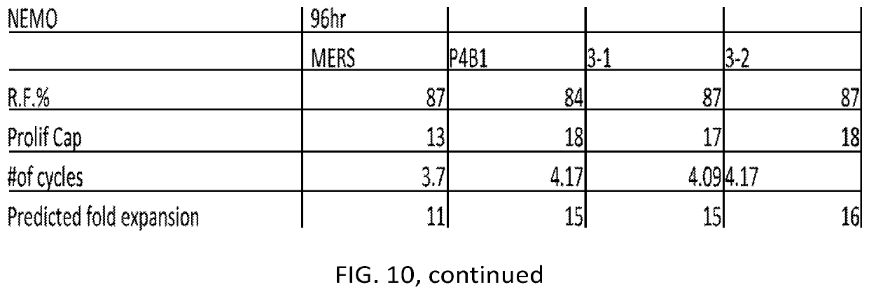

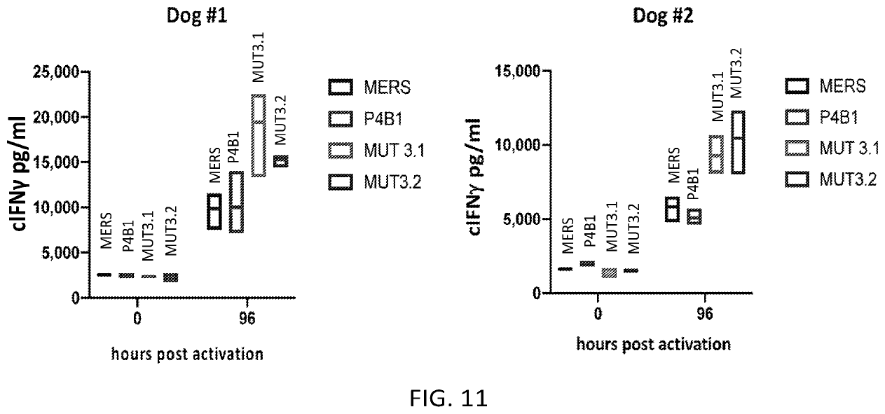

To determine whether P3C6 mutants were able to increase canine T cell proliferation and IFNγ production, canine PBMCs were labeled with Cell Trace Violet (CTV) and stimulated with the mitogen concanavalin A in the presence of the P3C6 mutant IgG4 molecules. The proliferation of CD5+ T cells was determined by flow cytometry (FIG. 10) and the production of IFNγ was measured by ELISA (FIG. 11) at 96 hours post stimulation.

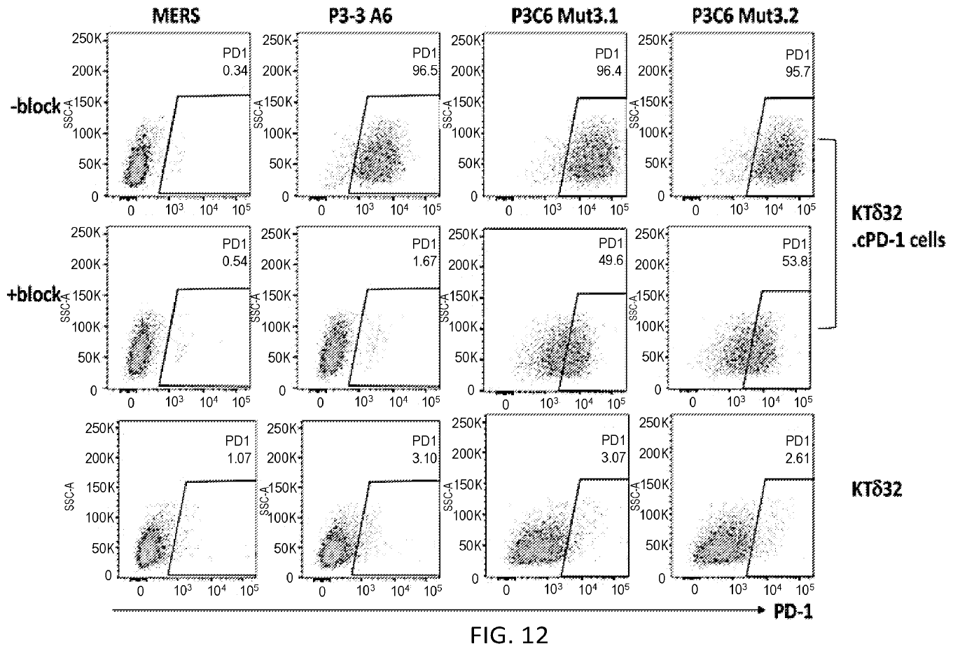

Given the relatively low number of unique scFv identified from the initial screen and the subsequent high throughput screen, a second high throughput screen was performed with 872 clones screened, and over 200 soluble scFvs clones present in expression extracts were identified as binding to cPD-1 by ELISA. These clones were further evaluated by flow cytometry for binding and 3 additional unique scFv clones were identified that bound to cPD-1 on the surface of KTδ32 cells. Only one of these clones from panning round 3, clone A6 was successfully reformatted as a full length canine IgG4. The ability of this full length clone to bind specifically to membrane bound cPD-1 was evaluated in flow cytometry (FIG. 12). Clone A6 specifically bound to KTδ32.cPD-1 cells, and this binding was blocked by pre-incubation of the IgG4 molecule with soluble cPD-1.

### ENUMERATED EMBODIMENTS

The following enumerated embodiments are provided, the numbering of which is not be construed as designating levels of importance.

Embodiment 1 provides an antibody or antigen-binding fragment thereof that specifically binds to canine programmed death protein 1 (cPD-1), wherein the antibody or antigen-binding fragment thereof comprises:
a heavy chain complementarity determining region 1 (HCDR1), comprising the amino acid sequence set forth in SEQ ID NOs: 1, 27, or 43,
a heavy chain complementarity determining region 2 (HCDR2), comprising the amino acid sequence set forth in SEQ ID NOs: 2, 28, or 44,
a heavy chain complementarity determining region 3 (HCDR3), comprising the amino acid sequence set forth in SEQ ID NOs: 3, 29, or 45,
a light chain complementarity determining region 1 (LCDR1), comprising the amino acid sequence set forth in SEQ ID NOs: 4, 30, or 46,
a light chain complementarity determining region 2 (LCDR2), comprising the amino acid sequence set forth in SEQ ID NOs: 5, 31, or 47, and
a light chain complementarity determining region 3 (LCDR3), comprising the amino acid sequence set forth in SEQ ID NOs: 6, 32, or 48.

Embodiment 2 provides the antibody or antigen-binding fragment thereof of embodiment 1, wherein the antibody or antigen-binding fragment thereof comprises:
a heavy chain variable region comprising an amino acid sequence having at least 80%, 85%, 90%, 95%, 96%, 96%, 97%, 98%, 99% identity to the amino acid sequence set forth in SEQ ID NOs: 7, 9, 33, or 49; and
a light chain variable region comprising an amino acid sequence having at least 80%, 85%, 90%, 95%, 96%, 97%, 98%, 99% identity to the amino acid sequence set forth in SEQ ID NOs: 11, 35, or 51.

Embodiment 3 provides the antibody or antigen-binding fragment thereof of embodiment 1 or 2, wherein the antibody or antigen-binding fragment thereof comprises:
an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, 23, 39, or 55, and
an immunoglobulin light chain, comprising an amino acid sequence having at least 80%, 85%, 90%, 95%, 96%, 97%, 98%, 99% identity to the amino acid sequence set forth in SEQ ID NOs: 25, 41, or 57.

Embodiment 4 provides an antibody or antigen-binding fragment thereof that specifically binds to canine programmed death protein 1 (cPD-1), wherein the antibody or antigen-binding fragment thereof comprises:
a heavy chain variable region comprising an amino acid sequence having at least 80%, 85%, 90%, 95%, 96%, 96%, 97%, 98%, 99% identity to the amino acid sequence set forth in SEQ ID NOs: 7, 9, 33, or 49; and
a light chain variable region comprising an amino acid sequence having at least 80%, 85%, 90%, 95%, 96%, 97%, 98%, 99% identity to the amino acid sequence set forth in SEQ ID NOs: 11, 35, or 51.

Embodiment 5 provides an antibody or antigen-binding fragment thereof that specifically binds to canine programmed death protein 1 (cPD-1), wherein the antibody or antigen-binding fragment thereof comprises:
a heavy chain variable region comprising the amino acid sequence set forth in SEQ ID NOs: 7, 9, 33, or 49; and
a light chain variable region comprising the amino acid sequence set forth in SEQ ID NOs: 11, 35, or 51.

Embodiment 6 provides an antibody or antigen-binding fragment thereof that specifically binds to canine programmed death protein 1 (cPD-1), wherein the antibody or antigen-binding fragment thereof comprises:
an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, 23, 39, or 55, and
an immunoglobulin light chain, comprising an amino acid sequence having at least 80%, 85%, 90%, 95%, 96%, 97%, 98%, 99% identity to the amino acid sequence set forth in SEQ ID NOs: 25, 41, or 57.

Embodiment 7 provides an antibody or antigen-binding fragment thereof that specifically binds to canine programmed death protein 1 (cPD-1), wherein the antibody or antigen-binding fragment thereof comprises:
an immunoglobulin heavy chain, comprising the amino acid sequence set forth in SEQ ID NOs: 19, 21, 23, 39, or 55, and
an immunoglobulin light chain, comprising the amino acid sequence set forth in SEQ ID NOs: 25, 41, or 57.

Embodiment 8 provides an antibody or antigen-binding fragment thereof that specifically binds to canine programmed death protein 1 (cPD-1), wherein the antibody or antigen-binding fragment thereof comprises:
a heavy chain complementarity determining region 1 (HCDR1), comprising the amino acid sequence set forth in SEQ ID NO: 1,
a heavy chain complementarity determining region 2 (HCDR2), comprising the amino acid sequence set forth in SEQ ID NO: 2,
a heavy chain complementarity determining region 3 (HCDR3), comprising the amino acid sequence set forth in SEQ ID NO: 3,
a light chain complementarity determining region 1 (LCDR1), comprising the amino acid sequence set forth in SEQ ID NO: 4,
a light chain complementarity determining region 2 (LCDR2), comprising the amino acid sequence set forth in SEQ ID NO: 5, and
a light chain complementarity determining region 3 (LCDR3), comprising the amino acid sequence set forth in SEQ ID NO: 6.

Embodiment 9 provides the antibody or antigen-binding fragment thereof of embodiment 8, wherein the antibody or antigen-binding fragment thereof comprises:
a heavy chain variable region comprising an amino acid sequence having at least 80%, 85%, 90%, 95%, 96%, 96%, 97%, 98%, 99% identity to the amino acid sequence set forth in SEQ ID NOs: 7 or 9; and
a light chain variable region comprising an amino acid sequence having at least 80%, 85%, 90%, 95%, 96%, 97%, 98%, 99% identity to the amino acid sequence set forth in SEQ ID NO: 11.

Embodiment 10 provides the antibody or antigen-binding fragment thereof of embodiment 8 or 9, wherein the antibody or antigen-binding fragment thereof comprises:
an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, or 23, and
an immunoglobulin light chain, comprising an amino acid sequence having at least 80%, 85%, 90%, 95%, 96%, 97%, 98%, 99% identity to the amino acid sequence set forth in SEQ ID NO: 25.

Embodiment 11 provides an antibody or antigen-binding fragment thereof that specifically binds to canine programmed death protein 1 (cPD-1), wherein the antibody or antigen-binding fragment thereof comprises:
a heavy chain variable region comprising an amino acid sequence having at least 80%, 85%, 90%, 95%, 96%, 96%, 97%, 98%, 99% identity to the amino acid sequence set forth in SEQ ID NOs: 7 or 9; and
a light chain variable region comprising an amino acid sequence having at least 80%, 85%, 90%, 95%, 96%, 97%, 98%, 99% identity to the amino acid sequence set forth in SEQ ID NO: 11.

Embodiment 12 provides an antibody or antigen-binding fragment thereof that specifically binds to canine programmed death protein 1 (cPD-1), wherein the antibody or antigen-binding fragment thereof comprises:
a heavy chain variable region comprising the amino acid sequence set forth in SEQ ID NO: 7; and
a light chain variable region comprising the amino acid sequence set forth in SEQ ID NO: 11.

Embodiment 13 provides an antibody or antigen-binding fragment thereof that specifically binds to canine programmed death protein 1 (cPD-1), wherein the antibody or antigen-binding fragment thereof comprises:
a heavy chain variable region comprising the amino acid sequence set forth in SEQ ID NO: 9; and
a light chain variable region comprising the amino acid sequence set forth in SEQ ID NO: 11.

Embodiment 14 provides an antibody or antigen-binding fragment thereof that specifically binds to canine programmed death protein 1 (cPD-1), wherein the antibody or antigen-binding fragment thereof comprises:
an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, or 23, and
an immunoglobulin light chain, comprising an amino acid sequence having at least 80%, 85%, 90%, 95%, 96%, 97%, 98%, 99% identity to the amino acid sequence set forth in SEQ ID NO: 25.

Embodiment 15 provides an antibody or antigen-binding fragment thereof that specifically binds to canine programmed death protein 1 (cPD-1), wherein the antibody or antigen-binding fragment thereof comprises:
an immunoglobulin heavy chain, comprising the amino acid sequence set forth in SEQ ID NOs: 19, and
an immunoglobulin light chain, comprising the amino acid sequence set forth in SEQ ID NO: 25.

Embodiment 16 provides an antibody or antigen-binding fragment thereof that specifically binds to canine programmed death protein 1 (cPD-1), wherein the antibody or antigen-binding fragment thereof comprises:
an immunoglobulin heavy chain, comprising the amino acid sequence set forth in SEQ ID NOs: 21, and
an immunoglobulin light chain, comprising the amino acid sequence set forth in SEQ ID NO: 25.

Embodiment 17 provides an antibody or antigen-binding fragment thereof that specifically binds to canine programmed death protein 1 (cPD-1), wherein the antibody or antigen-binding fragment thereof comprises:
an immunoglobulin heavy chain, comprising the amino acid sequence set forth in SEQ ID NOs: 23, and
an immunoglobulin light chain, comprising the amino acid sequence set forth in SEQ ID NO: 25.

Embodiment 18 provides an antibody or antigen-binding fragment thereof that specifically binds to canine programmed death protein 1 (cPD-1), wherein the antibody or antigen-binding fragment thereof comprises:
a heavy chain complementarity determining region 1 (HCDR1), comprising the amino acid sequence set forth in SEQ ID NO: 27,
a heavy chain complementarity determining region 2 (HCDR2), comprising the amino acid sequence set forth in SEQ ID NO: 28,
a heavy chain complementarity determining region 3 (HCDR3), comprising the amino acid sequence set forth in SEQ ID NO: 29,
a light chain complementarity determining region 1 (LCDR1), comprising the amino acid sequence set forth in SEQ ID NO: 30,
a light chain complementarity determining region 2 (LCDR2), comprising the amino acid sequence set forth in SEQ ID NO: 31, and
a light chain complementarity determining region 3 (LCDR3), comprising the amino acid sequence set forth in SEQ ID NO: 32.

Embodiment 19 provides the antibody or antigen-binding fragment thereof of embodiment 18, wherein the antibody or antigen-binding fragment thereof comprises:
a heavy chain variable region comprising an amino acid sequence having at least 80%, 85%, 90%, 95%, 96%, 96%, 97%, 98%, 99% identity to the amino acid sequence set forth in SEQ ID NO: 33; and
a light chain variable region comprising an amino acid sequence having at least 80%, 85%, 90%, 95%, 96%, 97%, 98%, 99% identity to the amino acid sequence set forth in SEQ ID NO: 35.

Embodiment 20 provides the antibody or antigen-binding fragment thereof of embodiment 18 or 19, wherein the antibody or antigen-binding fragment thereof comprises:
an immunoglobulin heavy chain, comprising an amino acid sequence having at least 80%, 85%, 90%, 95%, 96%, 97%, 98%, 99% identity to the amino acid sequence set forth in SEQ ID NO: 39, and
an immunoglobulin light chain, comprising an amino acid sequence having at least 80%, 85%, 90%, 95%, 96%, 97%, 98%, 99% identity to the amino acid sequence set forth in SEQ ID NO: 41.

Embodiment 21 provides an antibody or antigen-binding fragment thereof that specifically binds to canine programmed death protein 1 (cPD-1), wherein the antibody or antigen-binding fragment thereof comprises:
a heavy chain variable region comprising an amino acid sequence having at least 80%, 85%, 90%, 95%, 96%, 96%, 97%, 98%, 99% identity to the amino acid sequence set forth in SEQ ID NO: 33; and
a light chain variable region comprising an amino acid sequence having at least 80%, 85%, 90%, 95%, 96%, 97%, 98%, 99% identity to the amino acid sequence set forth in SEQ ID NO: 35.

Embodiment 22 provides antibody or antigen-binding fragment thereof that specifically binds to canine programmed death protein 1 (cPD-1), wherein the antibody or antigen-binding fragment thereof comprises:
a heavy chain variable region comprising the amino acid sequence set forth in SEQ ID NO: 33; and
a light chain variable region comprising the amino acid sequence set forth in SEQ ID NO: 35.

Embodiment 23 provides an antibody or antigen-binding fragment thereof that specifically binds to canine programmed death protein 1 (cPD-1), wherein the antibody or antigen-binding fragment thereof comprises:
an immunoglobulin heavy chain, comprising an amino acid sequence having at least 80%, 85%, 90%, 95%, 96%, 97%, 98%, 99% identity to the amino acid sequence set forth in SEQ ID NO: 39, and
an immunoglobulin light chain, comprising an amino acid sequence having at least 80%, 85%, 90%, 95%, 96%, 97%, 98%, 99% identity to the amino acid sequence set forth in SEQ ID NO: 41.

Embodiment 24 provides an antibody or antigen-binding fragment thereof that specifically binds to canine programmed death protein 1 (cPD-1), wherein the antibody or antigen-binding fragment thereof comprises:
an immunoglobulin heavy chain, comprising the amino acid sequence set forth in SEQ ID NO: 39, and
an immunoglobulin light chain, comprising the amino acid sequence set forth in SEQ ID NO: 41.

Embodiment 25 provides an antibody or antigen-binding fragment thereof that specifically binds to canine programmed death protein 1 (cPD-1), wherein the antibody or antigen-binding fragment thereof comprises:
a heavy chain complementarity determining region 1 (HCDR1), comprising the amino acid sequence set forth in SEQ ID NO: 43,
a heavy chain complementarity determining region 2 (HCDR2), comprising the amino acid sequence set forth in SEQ ID NO: 44,
a heavy chain complementarity determining region 3 (HCDR3), comprising the amino acid sequence set forth in SEQ ID NO: 45,
a light chain complementarity determining region 1 (LCDR1), comprising the amino acid sequence set forth in SEQ ID NO: 46,
a light chain complementarity determining region 2 (LCDR2), comprising the amino acid sequence set forth in SEQ ID NO: 47, and
a light chain complementarity determining region 3 (LCDR3), comprising the amino acid sequence set forth in SEQ ID NO: 48.

Embodiment 26 provides the antibody or antigen-binding fragment thereof of embodiment 25, wherein the antibody or antigen-binding fragment thereof comprises:
a heavy chain variable region comprising an amino acid sequence having at least 80%, 85%, 90%, 95%, 96%, 96%, 97%, 98%, 99% identity to the amino acid sequence set forth in SEQ ID NO: 49; and
a light chain variable region comprising an amino acid sequence having at least 80%, 85%, 90%, 95%, 96%, 97%, 98%, 99% identity to the amino acid sequence set forth in SEQ ID NO: 51.

Embodiment 27 provides the antibody or antigen-binding fragment thereof of embodiment 25 or 26, wherein the antibody or antigen-binding fragment thereof comprises:
an immunoglobulin heavy chain, comprising an amino acid sequence having at least 80%, 85%, 90%, 95%, 96%, 97%, 98%, 99% identity to the amino acid sequence set forth in SEQ ID NO: 55, and
an immunoglobulin light chain, comprising an amino acid sequence having at least 80%, 85%, 90%, 95%, 96%, 97%, 98%, 99% identity to the amino acid sequence set forth in SEQ ID NO: 57.

Embodiment 28 provides an antibody or antigen-binding fragment thereof that specifically binds to canine programmed death protein 1 (cPD-1), wherein the antibody or antigen-binding fragment thereof comprises:
a heavy chain variable region comprising an amino acid sequence having at least 80%, 85%, 90%, 95%, 96%, 96%, 97%, 98%, 99% identity to the amino acid sequence set forth in SEQ ID NO: 49; and
a light chain variable region comprising an amino acid sequence having at least 80%, 85%, 90%, 95%, 96%, 97%, 98%, 99% identity to the amino acid sequence set forth in SEQ ID NO: 51.

Embodiment 29 provides an antibody or antigen-binding fragment thereof that specifically binds to canine programmed death protein 1 (cPD-1), wherein the antibody or antigen-binding fragment thereof comprises:
a heavy chain variable region comprising the amino acid sequence set forth in SEQ ID NO: 49; and
a light chain variable region comprising the amino acid sequence set forth in SEQ ID NO: 51.

Embodiment 30 provides an antibody or antigen-binding fragment thereof that specifically binds to canine programmed death protein 1 (cPD-1), wherein the antibody or antigen-binding fragment thereof comprises:
an immunoglobulin heavy chain, comprising an amino acid sequence having at least 80%, 85%, 90%, 95%, 96%, 97%, 98%, 99% identity to the amino acid sequence set forth in SEQ ID NO: 55, and
an immunoglobulin light chain, comprising an amino acid sequence having at least 80%, 85%, 90%, 95%, 96%, 97%, 98%, 99% identity to the amino acid sequence set forth in SEQ ID NO: 57.

Embodiment 31 provides an antibody or antigen-binding fragment thereof that specifically binds to canine programmed death protein 1 (cPD-1), wherein the antibody or antigen-binding fragment thereof comprises:
an immunoglobulin heavy chain, comprising the amino acid sequence set forth in SEQ ID NO: 55, and
an immunoglobulin light chain, comprising the amino acid sequence set forth in SEQ ID NO: 57.

Embodiment 32 provides the antibody or antigen-binding fragment thereof of any one of embodiments 1-31, wherein the antibody or antigen-binding fragment thereof is an IgG, an IgM, an IgE, an IgA, or an IgD molecule, or is derived from one of these.

Embodiment 33 provides the antibody or antigen-binding fragment thereof of any one of embodiments 1-32, wherein the antibody or antigen-binding fragment thereof is selected from the group consisting of a full-length antibody, a Fab, a single-chain variable fragment (scFv), sc(Fv)2, dsFv, Fab, Fab′, (Fab′)2 and a diabody.

Embodiment 34 provides the antibody or antigen-binding fragment thereof of any one of embodiments 1-33, wherein the antibody is a full-length antibody.

Embodiment 35 provides the antibody or antigen-binding fragment thereof of any one of embodiments 1-34, wherein the antibody is a canine or caninized antibody.

Embodiment 36 provides the antibody or antigen-binding fragment thereof of any one of embodiments 1-35, wherein the antibody is a canine antibody.

Embodiment 37 provides the antibody or antigen-binding fragment thereof of any one of embodiments 1-33, wherein the antigen-binding fragment is an scFv.

Embodiment 38 provides the antibody or antigen-binding fragment thereof of any one of embodiments 1-37, wherein the specific binding of the antibody or antigen-binding fragment thereof to cPD-1 disrupts the interaction of cPD-1 and a ligand of cPD-1 (e.g., canine programmed death-ligand 1).

Embodiment 39 provides a canine antibody or antigen-binding fragment thereof that specifically binds to canine programmed death protein 1 (cPD-1).

Embodiment 40 provides a canine antibody or antigen-binding fragment thereof that specifically binds to canine programmed death protein 1 (cPD-1), comprising the antibody or antigen-binding fragment thereof of any one of embodiments 1-36.

Embodiment 41 provides the canine antibody or antigen-binding fragment thereof of embodiment 39 or 40, wherein the specific binding of the antibody or antigen-binding fragment thereof to cPD-1 disrupts the interaction of cPD-1 and a ligand of cPD-1 (e.g., canine programmed death-ligand 1).

Embodiment 42 provides a single-chain variable fragment (scFv) specifically binds canine programmed death protein 1 (cPD-1), comprising:
a heavy chain variable region that comprises heavy chain complementarity determining region 1 (HCDR1), HCDR2, and HCDR3, wherein HCDR1 comprises an amino acid sequence selected from the group consisting of SEQ ID NOs: 1, 27, and 43, HCDR2 comprises an amino acid sequence selected from the group consisting of SEQ ID NOs: 2, 28, and 44, and HCDR3 comprises an amino acid sequence selected from the group consisting of SEQ ID NOs: 3, 29, and 45; and
a light chain variable region that comprises light chain complementarity determining region 1 (LCDR1), LCDR2, and LCDR3, wherein LCDR1 comprises an amino acid sequence selected from the group consisting of SEQ ID NOs: 4, 30, and 46, LCDR2 comprises an amino acid sequence selected from the group consisting of SEQ ID NOs: 5, 31 and 47, and LCDR3 comprises an amino acid sequence selected from the group consisting of SEQ ID NOs: 6, 32, and 48,
wherein the heavy chain variable region and the light chain variable region are connected by a linker.

Embodiment 43 provides a single-chain variable fragment (scFv) specifically binds to canine programmed death protein 1 (cPD-1), comprising:
a heavy chain variable region comprising the amino acid sequence set forth in SEQ ID NOs: 7, 9, 33, or 49; and
a light chain variable region comprising the amino acid sequence set forth in SEQ ID NOs: 11, 35, or 51,
wherein the heavy chain variable region and the light chain variable region are connected by a linker.

Embodiment 44 provides a single chain variable fragment (scFv) specifically binds to canine programmed death protein 1 (cPD-1), comprising the amino acid sequence set forth in SEQ ID NOs: 13, 15, 17, 37, or 53.

Embodiment 45 provides the scFv of any one of embodiments 42-44, wherein the specific binding of the antibody or antigen-binding fragment thereof to cPD-1 disrupts the interaction of cPD-1 and a ligand of cPD-1 (e.g., canine programmed death-ligand 1).

Embodiment 46 provides a bispecific molecule comprising the antibody or antigen-binding fragment thereof of any one of embodiments 1-38, the canine antibody of any one of embodiments 39-41, or the scFv of any one of embodiments 42-45, linked to a molecule having a second binding specificity.

Embodiment 47 provides an immunoconjugate comprising the antibody or antigen-binding fragment thereof of any one of embodiments 1-38, the canine antibody of any one of embodiments 39-41, or the scFv of any one of embodiments 42-45, or the bispecific molecule of embodiment 46, linked to a therapeutic agent.

Embodiment 48 provides an isolated nucleic acid encoding the antibody or antigen-binding fragment thereof of any one of embodiments 1-38, the canine antibody of any one of embodiments 39-41, or the scFv of any one of embodiments 42-45.

Embodiment 49 provides an isolated nucleic acid encoding an antibody or antigen-binding fragment thereof, wherein the antibody or antigen-binding fragment thereof comprises:
a heavy chain variable region encoded by a nucleic acid comprising a polynucleotide sequence having at least 80%, 85%, 90%, 95%, 96%, 96%, 97%, 98%, 99% identity to the polynucleotide sequence set forth in SEQ ID NOs: 8, 10, 34, or 50; and
a light chain variable region encoded by a nucleic acid comprising a polynucleotide sequence having at least 80%, 85%, 90%, 95%, 96%, 96%, 97%, 98%, 99% identity to the polynucleotide sequence set forth in SEQ ID NOs: 12, 36, or 52;

Embodiment 50 provides the isolated nucleic acid of embodiment 49, wherein the heavy chain variable region is encoded by a nucleic acid comprising the polynucleotide sequence set forth in SEQ ID NOs: 8, 10, 34, or 50.

Embodiment 51 provides the isolated nucleic acid of embodiment 49 or 50, wherein the light chain variable region is encoded by a nucleic acid comprising the polynucleotide sequence set forth in SEQ ID NOs: 12, 36, or 52.

Embodiment 52 provides an isolated nucleic acid encoding an antibody or antigen binding fragment thereof comprising:
a heavy chain variable region encoded by a nucleic acid sequence comprising the polynucleotide sequence set forth in SEQ ID NO: 8; and
a light chain variable region encoded by a nucleic acid sequence comprising the polynucleotide sequence set forth in SEQ ID NO: 12.

Embodiment 53 provides an isolated nucleic acid encoding an antibody or antigen binding fragment thereof comprising:
a heavy chain variable region encoded by a nucleic acid sequence comprising the polynucleotide sequence set forth in SEQ ID NO: 10; and
a light chain variable region encoded by a nucleic acid sequence comprising the polynucleotide sequence set forth in SEQ ID NO: 12.

Embodiment 54 provides an isolated nucleic acid encoding an antibody or antigen binding fragment thereof comprising:
a heavy chain variable region encoded by a nucleic acid sequence comprising the polynucleotide sequence set forth in SEQ ID NO: 34; and
a light chain variable region encoded by a nucleic acid sequence comprising the polynucleotide sequence set forth in SEQ ID NO: 36.

Embodiment 55 provides an isolated nucleic acid encoding an antibody or antigen binding fragment thereof comprising:
a heavy chain variable region encoded by a nucleic acid sequence comprising the polynucleotide sequence set forth in SEQ ID NO: 50; and
a light chain variable region encoded by a nucleic acid sequence comprising the polynucleotide sequence set forth in SEQ ID NO: 52.

Embodiment 56 provides the isolated nucleic acid of any one of embodiments 49-55, wherein the antibody or antigen-binding fragment thereof is an IgG, an IgM, an IgE, an IgA, or an IgD molecule, or is derived from one of these.

Embodiment 57 provides the isolated nucleic acid of any one of embodiments 49-56, wherein the antibody or antigen-binding fragment thereof is selected from the group consisting of a full-length antibody, a Fab, a single-chain variable fragment (scFv), sc(Fv)2, dsFv, Fab, Fab′, (Fab′)2 and a diabody.

Embodiment 58 provides the isolated nucleic acid of any one of embodiments 49-57, wherein the antibody is a full-length antibody.

Embodiment 59 provides the isolated nucleic acid of any one of embodiments 49-58, wherein the antibody is a canine or caninized antibody.

Embodiment 60 provides the isolated nucleic acid of any one of embodiments 49-59, wherein the antibody is a canine antibody.

Embodiment 61 provides an isolated nucleic acid encoding a single-chain variable fragment (scFv) comprising:
a heavy chain variable region comprising the nucleotide sequence set forth in SEQ ID NOs: 8, 10, 34, or 50; and
a light chain variable region comprising the nucleotide sequence set forth in SEQ ID NOs: 12, 36, or 52,
wherein the heavy chain variable region and the light chain variable region are connected by a linker.

Embodiment 62 provides an isolated nucleic acid encoding a single-chain variable fragment (scFv) comprising the polynucleotide sequence set forth in SEQ ID NOs: 14, 16, 18, 38, or 54.

Embodiment 63 provides the isolated nucleic acid of any one of embodiments 49-62, wherein the antibody or antigen-binding fragment thereof or the scFv specifically binds to canine programmed death protein 1 (cPD-1).

Embodiment 64 provides the isolated nucleic acid of embodiment 56, wherein the specific binding of the antibody or antigen-binding fragment thereof or the scFv to cPD-1 disrupts the interaction of cPD-1 and a ligand of cPD-1 (e.g., canine programmed death-ligand 1).

Embodiment 65 provides a vector comprising the isolated nucleic acid of any one of embodiments 48-64.

Embodiment 66 provides the vector of embodiment 65, wherein the vector is an expression vector.

Embodiment 67 provides the vector of embodiment 65 or 66, wherein the vector is selected from the group consisting of a DNA vector, an RNA vector, a plasmid, a lentiviral vector, an adenoviral vector, an adeno-associated viral vector, and a retroviral vector.

Embodiment 68 provides a host cell comprising the isolated nucleic acid of any one of embodiments 48-64 or the vector of any one of embodiments 65-67.

Embodiment 69 provides the host cell of embodiment 68, wherein the host cell is of eukaryotic or prokaryotic origin.

Embodiment 70 provides the host cell of embodiment 68 or 69, wherein the host cell is of mammalian origin.

Embodiment 71 provides the host cell of embodiment 68 or 69, wherein the host cell is of bacterial origin.

Embodiment 72 provides the host cell of any one of embodiments 68-70, wherein the host cell is a Chinese Hamster Ovary cell.

Embodiment 73 provides a pharmaceutical composition comprising the antibody or antigen-binding fragment thereof of any one of embodiments 1-38, the canine antibody of any one of embodiments 39-41, or the scFv of any one of embodiments 42-45, the bispecific molecule of embodiment 46, or the immunoconjugate of embodiment 47, and a pharmaceutically acceptable excipient, carrier, or diluent.

Embodiment 74 provides the pharmaceutical composition of embodiment 73, formulated for intravenous administration.

Embodiment 75 provides pharmaceutical composition of embodiment 73, formulated for subcutaneous administration.

Embodiment 76 provides the pharmaceutical composition of embodiment 73, formulated for intratumoral administration.

Embodiment 77 provides a kit comprising the antibody or antigen-binding fragment thereof of any one of embodiments 1-38, the canine antibody of any one of embodiments 39-41, or the scFv of any one of embodiments 42-45, the bispecific molecule of embodiment 46, the immunoconjugate of embodiment 47, or the pharmaceutical composition of embodiment 73, and an instruction for use.

Embodiment 78 provides a method for increasing T cell proliferation and reducing T cell exhaustion in a subject in need thereof, comprising administering to the subject the antibody or antigen-binding fragment thereof of any one of embodiments 1-38, the canine antibody of any one of embodiments 39-41, or the scFv of any one of embodiments 42-45, the bispecific molecule of embodiment 46, or the immunoconjugate of embodiment 47, or the pharmaceutical composition of embodiment 73.

Embodiment 79 provides a method for increasing IFNγ production in a subject in need thereof, comprising administering to the subject the antibody or antigen-binding fragment thereof of any one of embodiments 1-38, the canine antibody of any one of embodiments 39-41, or the scFv of any one of embodiments 42-45, the bispecific molecule of embodiment 46, or the immunoconjugate of embodiment 47, or the pharmaceutical composition of embodiment 73.

Embodiment 80 provides a method for inhibiting canine programmed death protein 1 (cPD-1) signaling pathway in a subject in need thereof, comprising administering to the subject the antibody or antigen-binding fragment thereof of any one of embodiments 1-38, the canine antibody of any one of embodiments 39-41, or the scFv of any one of embodiments 42-45, the bispecific molecule of embodiment 46, or the immunoconjugate of embodiment 47, or the pharmaceutical composition of embodiment 73.

Embodiment 81 provides the method of any one of embodiments 78-80, wherein the subject has cancer, sepsis or septic shock, or a chronic infection (e.g., viral infections).

Embodiment 82 provides a method for treating a disease or condition in a subject in need thereof, comprising administering to the subject the antibody or antigen-binding fragment thereof of any one of embodiments 1-38, the canine antibody of any one of embodiments 39-41, or the scFv of any one of embodiments 42-45, the bispecific molecule of embodiment 46, or the immunoconjugate of embodiment 47, or the pharmaceutical composition of embodiment 73.

Embodiment 83 provides the method of embodiment 82, wherein the disease or condition comprises cancer, sepsis or septic shock, or a chronic infection (e.g., viral infections).

Embodiment 84 provides the method of any one of embodiments 78-83, further comprising administering one or more additional therapeutics (e.g., chemotherapy) or interventions.

Embodiment 85 provides the method of any one of embodiments 81, 83, or 84, wherein the cancer is associated with canine programmed death protein 1 (cPD-1) signaling pathway.

Embodiment 86 provides the method of embodiment 85, wherein a ligand for cPD-1 (e.g., canine programmed death-ligand 1) is expressed on a cancer cell of the subject.

Embodiment 87 provides the method of any one of embodiments 80-86, wherein cPD-1 is expressed on an immune cell of the subject.

Embodiment 88 provides the method of embodiment 87, wherein the immune cell comprises a T cell (e.g., an activated T cell), a B cell, a Natural Killer (NK) cell, a regulatory T cell, a macrophage, a Dendritic cell (DC), or a tumor-infiltrating lymphocyte.

Embodiment 89 provides the method of any one of embodiments 81-88, wherein the cancer comprises a melanoma (e.g., metastatic malignant melanoma), a prostate cancer (for example hormone refractory prostate adenocarcinoma), a head and neck cancer (for example, squamous cell carcinoma of the head and neck), a cervical cancer, a thyroid cancer, a glioblastoma, a glioma, leukemia, a lymphoma (for example, a B cell lymphoma), an adrenal gland cancer, an AIDS-associated cancer, an alveolar soft part sarcoma, an astrocytic tumor, bone cancer, a brain and spinal cord cancer, a metastatic brain tumor, a carotid body tumor, a chondrosarcoma, a chordoma, a chromophobe renal cell carcinoma, a clear cell carcinoma, cutaneous benign fibrous histiocytoma, a desmoplastic small round cell tumor, an ependymoma, a Ewing's tumor, an extra skeletal myxoid chondrosarcoma, a fibrogenesis imperfecta ossium, a fibrous dysplasia of the bone, a gallbladder or bile duct cancer, a gestational trophoblastic disease, a germ cell tumor, a hematological malignancy, hepatocellular carcinoma, an islet cell tumor, a Kaposi's sarcoma, a kidney cancer, a lipoma/benign lipomatous tumor, a liposarcoma/malignant lipomatous tumor, a medulloblastoma, a meningioma, a Merkel cell carcinoma, a multiple endocrine neoplasia, a multiple myeloma, a myelodysplasia syndrome, a neuroblastoma, a neuroendocrine tumor, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomyosarcoma, a sarcoma, a soft-tissue sarcoma, a squamous cell cancer, a stomach cancer, a synovial sarcoma, a testicular cancer, a thymic carcinoma, a thymoma, a thyroid metastatic cancer, a uterine cancer, or any combination thereof.

Embodiment 90 provides the method of any one of embodiments 81-88, wherein the cancer comprises cervical cancer, lung cancers, liver cancers, ovarian cancers, cancers of the skin including melanoma and squamous cell carcinoma, colon cancer, bladder cancer, breast cancer, kidney cancer, esophageal cancer, stomach cancer, pancreatic cancers, head cancer, and neck cancer.

Embodiment 91 provides the method of any one of embodiments 78-90, wherein the antibody or antigen-binding fragment thereof, the canine antibody, the scFv, the bispecific molecule, or the immunoconjugate specifically binds to cPD-1.

Embodiment 92 provides the method of embodiment 91, wherein the binding of antibody or antigen-binding fragment thereof, the canine antibody, the scFv, the bispecific molecule, or the immunoconjugate to cPD-1 disrupts the interaction of cPD-1 and a ligand of cPD-1 (e.g., canine programmed death-ligand 1).

Embodiment 93 provides the method of any one of embodiments 78-92, wherein the subject is canine.

Embodiment 94 provides a method of making the pharmaceutical composition of embodiment of embodiment 73, comprising admixing the antibody or antigen-binding fragment thereof of any one of embodiments 1-38, the canine antibody of any one of embodiments 39-41, or the scFv of any one of embodiments 42-45, the bispecific molecule of embodiment 46, or the immunoconjugate of embodiment 47, and a pharmaceutically acceptable excipient, carrier, or diluent.

Embodiment 95 provides a method of making a composition for treating cancer, sepsis or septic shock, or chronic infection (e.g., viral infections), comprising admixing the antibody or antigen-binding fragment thereof of any one of embodiments 1-38, the canine antibody of any one of embodiments 39-41, or the scFv of any one of embodiments 42-45, the bispecific molecule of embodiment 46, or the immunoconjugate of embodiment 47 and a pharmaceutically acceptable excipient, carrier, or diluent.

Embodiment 96 provides the method of embodiment 95, wherein the cancer comprises a melanoma (e.g., metastatic malignant melanoma), a prostate cancer (for example hormone refractory prostate adenocarcinoma), a head and neck cancer (for example, squamous cell carcinoma of the head and neck), a cervical cancer, a thyroid cancer, a glioblastoma, a glioma, leukemia, a lymphoma (for example, a B cell lymphoma), an adrenal gland cancer, an AIDS-associated cancer, an alveolar soft part sarcoma, an astrocytic tumor, bone cancer, a brain and spinal cord cancer, a metastatic brain tumor, a carotid body tumor, a chondrosarcoma, a chordoma, a chromophobe renal cell carcinoma, a clear cell carcinoma, cutaneous benign fibrous histiocytoma, a desmoplastic small round cell tumor, an ependymoma, a Ewing's tumor, an extra skeletal myxoid chondrosarcoma, a fibrogenesis imperfecta ossium, a fibrous dysplasia of the bone, a gallbladder or bile duct cancer, a gestational trophoblastic disease, a germ cell tumor, a hematological malignancy, hepatocellular carcinoma, an islet cell tumor, a Kaposi's sarcoma, a kidney cancer, a lipoma/benign lipomatous tumor, a liposarcoma/malignant lipomatous tumor, a medulloblastoma, a meningioma, a Merkel cell carcinoma, a multiple endocrine neoplasia, a multiple myeloma, a myelodysplasia syndrome, a neuroblastoma, a neuroendocrine tumor, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomyosarcoma, a sarcoma, a soft-tissue sarcoma, a squamous cell cancer, a stomach cancer, a synovial sarcoma, a testicular cancer, a thymic carcinoma, a thymoma, a thyroid metastatic cancer, a uterine cancer, or any combination thereof.

Embodiment 97 provides the method of embodiment 95, wherein the cancer comprises cervical cancer, lung cancers, liver cancers, ovarian cancers, cancers of the skin including melanoma and squamous cell carcinoma, colon cancer, bladder cancer, breast cancer, kidney cancer, esophageal cancer, stomach cancer, pancreatic cancers, head cancer, and neck cancer.

Embodiment 98 provides the antibody or antigen-binding fragment thereof of any one of embodiments 1-38, the canine antibody of any one of embodiments 39-41, or the scFv of any one of embodiments 42-45, the bispecific molecule of embodiment 46, the immunoconjugate of embodiment 47, or the pharmaceutical composition of embodiment 73, for use as a medicament.

Embodiment 99 provides the antibody or antigen-binding fragment thereof of any one of embodiments 1-38, the canine antibody of any one of embodiments 39-41, or the scFv of any one of embodiments 42-45, the bispecific molecule of embodiment 46, the immunoconjugate of embodiment 47, or the pharmaceutical composition of embodiment 73, for use as a medicament for the treatment of cancer, sepsis or septic shock, or chronic infection (e.g., viral infections).

Embodiment 100 provides use of the antibody or antigen-binding fragment thereof of any one of embodiments 1-38, the canine antibody of any one of embodiments 39-41, or the scFv of any one of embodiments 42-45, the bispecific molecule of embodiment 46, the immunoconjugate of embodiment 47, or the pharmaceutical composition of embodiment 73 for the manufacture of a medicament.

Embodiment 101 provides use of the antibody or antigen-binding fragment thereof of any one of embodiments 1-38, the canine antibody of any one of embodiments 39-41, or the scFv of any one of embodiments 42-45, the bispecific molecule of embodiment 46, the immunoconjugate of embodiment 47, or the pharmaceutical composition of embodiment 73 for the manufacture of a medicament for the treatment of cancer, sepsis or septic shock, or chronic infection (e.g., viral infections).

Embodiment 102 provides a method of producing the antibody or antigen-binding fragment thereof of any one of embodiments 1-38, the canine antibody of any one of embodiments 39-41, or the scFv of any one of embodiments 42-45, the method comprising culturing the host cell of any one of embodiments 68-72.

Embodiment 103 provides the method of embodiment 102, further comprises incubating the host cell of any one of embodiments 68-72 in a cell culture medium under conditions sufficient to allow expression and secretion of the antibody or antigen-binding fragment thereof of any one of embodiments 1-38, the canine antibody of any one of embodiments 39-41, or the scFv of any one of embodiments 42-45.

### OTHER EMBODIMENTS

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this disclosure has been disclosed with reference to specific embodiments, it is apparent that other embodiments and variations of this disclosure can be devised by others skilled in the art without departing from the true spirit and scope of the present disclosure. The appended claims are intended to be construed to include all such embodiments and equivalent variations.

What is claimed:

1. An antibody or antigen-binding fragment thereof that specifically binds to canine programmed death protein 1 (cPD-1), wherein the antibody or antigen-binding fragment thereof comprises:
(a) a heavy chain complementarity determining region 1 (HCDR1), comprising the amino acid sequence set forth in SEQ ID NOs: 1, 27, or 43,
(b) a heavy chain complementarity determining region 2 (HCDR2), comprising the amino acid sequence set forth in SEQ ID NOs: 2, 28, or 44,
(c) a heavy chain complementarity determining region 3 (HCDR3), comprising the amino acid sequence set forth in SEQ ID NOs: 3, 29, or 45,
(d) a light chain complementarity determining region 1 (LCDR1), comprising the amino acid sequence set forth in SEQ ID NOs: 4, 30, or 46,
(e) a light chain complementarity determining region 2 (LCDR2), comprising the amino acid sequence set forth in SEQ ID NOs: 5, 31, or 47, and
(f) a light chain complementarity determining region 3 (LCDR3), comprising the amino acid sequence set forth in SEQ ID NOs: 6, 32, or 48.

2. The antibody or antigen-binding fragment thereof of claim 1, wherein the antibody or antigen-binding fragment thereof comprises:
(a) a heavy chain variable region comprising an amino acid sequence having at least 80%, 85%, 90%, 95%, 96%, 96%, 97%, 98%, 99% identity to the amino acid sequence set forth in SEQ ID NOs: 7, 9, 33, or 49; and
(b) a light chain variable region comprising an amino acid sequence having at least 80%, 85%, 90%, 95%, 96%, 97%, 98%, 99% identity to the amino acid sequence set forth in SEQ ID NOs: 11, 35, or 51.

3. The antibody or antigen-binding fragment thereof of claim 1, wherein the antibody or antigen-binding fragment thereof comprises:
(a) an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, 23, 39, or 55, and
(b) an immunoglobulin light chain, comprising an amino acid sequence having at least 80%, 85%, 90%, 95%, 96%, 97%, 98%, 99% identity to the amino acid sequence set forth in SEQ ID NOs: 25, 41, or 57.

4. An antibody or antigen-binding fragment thereof that specifically binds to canine programmed death protein 1 (cPD-1), wherein the antibody or antigen-binding fragment thereof comprises:
(a) a heavy chain variable region comprising an amino acid sequence having at least 80%, 85%, 90%, 95%, 96%, 96%, 97%, 98%, 99% identity to the amino acid sequence set forth in SEQ ID NOs: 7, 9, 33, or 49; and
(b) a light chain variable region comprising an amino acid sequence having at least 80%, 85%, 90%, 95%, 96%, 97%, 98%, 99% identity to the amino acid sequence set forth in SEQ ID NOs: 11, 35, or 51.

5. An antibody or antigen-binding fragment thereof that specifically binds to canine programmed death protein 1 (cPD-1), wherein the antibody or antigen-binding fragment thereof comprises:
(a) a heavy chain variable region comprising the amino acid sequence set forth in SEQ ID NOs: 7, 9, 33, or 49; and
(b) a light chain variable region comprising the amino acid sequence set forth in SEQ ID NOs: 11, 35, or 51.

6. An antibody or antigen-binding fragment thereof that specifically binds to canine programmed death protein 1 (cPD-1), wherein the antibody or antigen-binding fragment thereof comprises:
(a) an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, 23, 39, or 55, and
(b) an immunoglobulin light chain, comprising an amino acid sequence having at least 80%, 85%, 90%, 95%, 96%, 97%, 98%, 99% identity to the amino acid sequence set forth in SEQ ID NOs: 25, 41, or 57.

7. An antibody or antigen-binding fragment thereof that specifically binds to canine programmed death protein 1 (cPD-1), wherein the antibody or antigen-binding fragment thereof comprises:
(a) an immunoglobulin heavy chain, comprising the amino acid sequence set forth in SEQ ID NOs: 19, 21, 23, 39, or 55, and
(b) an immunoglobulin light chain, comprising the amino acid sequence set forth in SEQ ID NOs: 25, 41, or 57.

8. An antibody or antigen-binding fragment thereof that specifically binds to canine programmed death protein 1 (cPD-1), wherein the antibody or antigen-binding fragment thereof comprises:
(a) a heavy chain complementarity determining region 1 (HCDR1), comprising the amino acid sequence set forth in SEQ ID NO: 1,
(b) a heavy chain complementarity determining region 2 (HCDR2), comprising the amino acid sequence set forth in SEQ ID NO: 2,
(c) a heavy chain complementarity determining region 3 (HCDR3), comprising the amino acid sequence set forth in SEQ ID NO: 3,
(d) a light chain complementarity determining region 1 (LCDR1), comprising the amino acid sequence set forth in SEQ ID NO: 4,
(e) a light chain complementarity determining region 2 (LCDR2), comprising the amino acid sequence set forth in SEQ ID NO: 5, and
(f) a light chain complementarity determining region 3 (LCDR3), comprising the amino acid sequence set forth in SEQ ID NO: 6.

9. The antibody or antigen-binding fragment thereof of claim 8, wherein the antibody or antigen-binding fragment thereof comprises:
(a) a heavy chain variable region comprising an amino acid sequence having at least 80%, 85%, 90%, 95%, 96%, 96%, 97%, 98%, 99% identity to the amino acid sequence set forth in SEQ ID NOs: 7 or 9; and
(b) a light chain variable region comprising an amino acid sequence having at least 80%, 85%, 90%, 95%, 96%, 97%, 98%, 99% identity to the amino acid sequence set forth in SEQ ID NO: 11.

10. The antibody or antigen-binding fragment thereof of claim 8, wherein the antibody or antigen-binding fragment thereof comprises:
(a) an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, or 23, and
(b) an immunoglobulin light chain, comprising an amino acid sequence having at least 80%, 85%, 90%, 95%, 96%, 97%, 98%, 99% identity to the amino acid sequence set forth in SEQ ID NO: 25.

11. An antibody or antigen-binding fragment thereof that specifically binds to canine programmed death protein 1 (cPD-1), wherein the antibody or antigen-binding fragment thereof comprises:
(a) a heavy chain variable region comprising an amino acid sequence having at least 80%, 85%, 90%, 95%, 96%, 96%, 97%, 98%, 99% identity to the amino acid sequence set forth in SEQ ID NOs: 7 or 9; and
(b) a light chain variable region comprising an amino acid sequence having at least 80%, 85%, 90%, 95%, 96%, 97%, 98%, 99% identity to the amino acid sequence set forth in SEQ ID NO: 11.

12. An antibody or antigen-binding fragment thereof that specifically binds to canine programmed death protein 1 (cPD-1), wherein the antibody or antigen-binding fragment thereof comprises:
(a) a heavy chain variable region comprising the amino acid sequence set forth in SEQ ID NO: 7; and a light chain variable region comprising the amino acid sequence set forth in SEQ ID NO: 11; or
(b) a heavy chain variable region comprising the amino acid sequence set forth in SEQ ID NO: 9 and a light chain variable region comprising the amino acid sequence set forth in SEQ ID NO: 11;

13. (canceled)

14. An antibody or antigen-binding fragment thereof that specifically binds to canine programmed death protein 1 (cPD-1), wherein the antibody or antigen-binding fragment thereof comprises:
(a) an immunoglobulin heavy chain, comprising an amino acid sequence having at least 80%, 85%, 90%, 95%, 96%, 97%, 98%, 99% identity to the amino acid sequence set forth in SEQ ID NOs: 19, 21, or 23, and
(b) an immunoglobulin light chain, comprising an amino acid sequence having at least 80%, 85%, 90%, 95%, 96%, 97%, 98%, 99% identity to the amino acid sequence set forth in SEQ ID NO: 25.

15. An antibody or antigen-binding fragment thereof that specifically binds to canine programmed death protein 1 (cPD-1), wherein the antibody or antigen-binding fragment thereof comprises:
(a) an immunoglobulin heavy chain, comprising the amino acid sequence set forth in SEQ ID NOs: 19, and an immunoglobulin light chain, comprising the amino acid sequence set forth in SEQ ID NO: 25;
(b) an immunoglobulin heavy chain, comprising the amino acid sequence set forth in SEQ ID NOs: 21, and an immunoglobulin light chain, comprising the amino acid sequence set forth in SEQ ID NO: 25; or
(c) an immunoglobulin heavy chain, comprising the amino acid sequence set forth in SEQ ID NOs: 23, and an immunoglobulin light chain, comprising the amino acid sequence set forth in SEQ ID NO: 25.

16.-17. (canceled)

18. An antibody or antigen-binding fragment thereof that specifically binds to canine programmed death protein 1 (cPD-1), wherein the antibody or antigen-binding fragment thereof comprises:
(a) a heavy chain complementarity determining region 1 (HCDR1), comprising the amino acid sequence set forth in SEQ ID NO: 27,
(b) a heavy chain complementarity determining region 2 (HCDR2), comprising the amino acid sequence set forth in SEQ ID NO: 28,
(c) a heavy chain complementarity determining region 3 (HCDR3), comprising the amino acid sequence set forth in SEQ ID NO: 29,
(d) a light chain complementarity determining region 1 (LCDR1), comprising the amino acid sequence set forth in SEQ ID NO: 30,
(e) a light chain complementarity determining region 2 (LCDR2), comprising the amino acid sequence set forth in SEQ ID NO: 31, and
(f) a light chain complementarity determining region 3 (LCDR3), comprising the amino acid sequence set forth in SEQ ID NO: 32.

19. The antibody or antigen-binding fragment thereof of claim 18, wherein the antibody or antigen-binding fragment thereof comprises:
(a) a heavy chain variable region comprising an amino acid sequence having at least 80%, 85%, 90%, 95%, 96%, 96%, 97%, 98%, 99% identity to the amino acid sequence set forth in SEQ ID NO: 33; and
(b) a light chain variable region comprising an amino acid sequence having at least 80%, 85%, 90%, 95%, 96%, 97%, 98%, 99% identity to the amino acid sequence set forth in SEQ ID NO: 35.

20. The antibody or antigen-binding fragment thereof of claim 18, wherein the antibody or antigen-binding fragment thereof comprises:
(a) an immunoglobulin heavy chain, comprising an amino acid sequence having at least 80%, 85%, 90%, 95%, 96%, 97%, 98%, 99% identity to the amino acid sequence set forth in SEQ ID NO: 39, and
(b) an immunoglobulin light chain, comprising an amino acid sequence having at least 80%, 85%, 90%, 95%, 96%, 97%, 98%, 99% identity to the amino acid sequence set forth in SEQ ID NO: 41.

21. An antibody or antigen-binding fragment thereof that specifically binds to canine programmed death protein 1 (cPD-1), wherein the antibody or antigen-binding fragment thereof comprises:
(a) a heavy chain variable region comprising an amino acid sequence having at least 80%, 85%, 90%, 95%, 96%, 96%, 97%, 98%, 99% identity to the amino acid sequence set forth in SEQ ID NO: 33; and
(b) a light chain variable region comprising an amino acid sequence having at least 80%, 85%, 90%, 95%, 96%, 97%, 98%, 99% identity to the amino acid sequence set forth in SEQ ID NO: 35.

22. An antibody or antigen-binding fragment thereof that specifically binds to canine programmed death protein 1 (cPD-1), wherein the antibody or antigen-binding fragment thereof comprises:
(a) a heavy chain variable region comprising the amino acid sequence set forth in SEQ ID NO: 33; and
(b) a light chain variable region comprising the amino acid sequence set forth in SEQ ID NO: 35.

23. An antibody or antigen-binding fragment thereof that specifically binds to canine programmed death protein 1 (cPD-1), wherein the antibody or antigen-binding fragment thereof comprises:
(a) an immunoglobulin heavy chain, comprising an amino acid sequence having at least 80%, 85%, 90%, 95%, 96%, 97%, 98%, 99% identity to the amino acid sequence set forth in SEQ ID NO: 39, and
(b) an immunoglobulin light chain, comprising an amino acid sequence having at least 80%, 85%, 90%, 95%, 96%, 97%, 98%, 99% identity to the amino acid sequence set forth in SEQ ID NO: 41.

24. An antibody or antigen-binding fragment thereof that specifically binds to canine programmed death protein 1 (cPD-1), wherein the antibody or antigen-binding fragment thereof comprises:
(a) an immunoglobulin heavy chain, comprising the amino acid sequence set forth in SEQ ID NO: 39, and
(b) an immunoglobulin light chain, comprising the amino acid sequence set forth in SEQ ID NO: 41.

25. An antibody or antigen-binding fragment thereof that specifically binds to canine programmed death protein 1 (cPD-1), wherein the antibody or antigen-binding fragment thereof comprises:
(a) a heavy chain complementarity determining region 1 (HCDR1), comprising the amino acid sequence set forth in SEQ ID NO: 43,
(b) a heavy chain complementarity determining region 2 (HCDR2), comprising the amino acid sequence set forth in SEQ ID NO: 44,
(c) a heavy chain complementarity determining region 3 (HCDR3), comprising the amino acid sequence set forth in SEQ ID NO: 45,
(d) a light chain complementarity determining region 1 (LCDR1), comprising the amino acid sequence set forth in SEQ ID NO: 46,
(e) a light chain complementarity determining region 2 (LCDR2), comprising the amino acid sequence set forth in SEQ ID NO: 47, and
(f) a light chain complementarity determining region 3 (LCDR3), comprising the amino acid sequence set forth in SEQ ID NO: 48.

26. The antibody or antigen-binding fragment thereof of claim 25, wherein the antibody or antigen-binding fragment thereof comprises:
(a) a heavy chain variable region comprising an amino acid sequence having at least 80%, 85%, 90%, 95%, 96%, 96%, 97%, 98%, 99% identity to the amino acid sequence set forth in SEQ ID NO: 49; and
(b) a light chain variable region comprising an amino acid sequence having at least 80%, 85%, 90%, 95%, 96%, 97%, 98%, 99% identity to the amino acid sequence set forth in SEQ ID NO: 51.

27. The antibody or antigen-binding fragment thereof of claim 25, wherein the antibody or antigen-binding fragment thereof comprises:
(a) an immunoglobulin heavy chain, comprising an amino acid sequence having at least 80%, 85%, 90%, 95%, 96%, 97%, 98%, 99% identity to the amino acid sequence set forth in SEQ ID NO: 55, and
(b) an immunoglobulin light chain, comprising an amino acid sequence having at least 80%, 85%, 90%, 95%, 96%, 97%, 98%, 99% identity to the amino acid sequence set forth in SEQ ID NO: 57.

28. (canceled)

29. An antibody or antigen-binding fragment thereof that specifically binds to canine programmed death protein 1 (cPD-1), wherein the antibody or antigen-binding fragment thereof comprises:
(a) a heavy chain variable region comprising the amino acid sequence set forth in SEQ ID NO: 49; and
(b) a light chain variable region comprising the amino acid sequence set forth in SEQ ID NO: 51.

30. An antibody or antigen-binding fragment thereof that specifically binds to canine programmed death protein 1 (cPD-1), wherein the antibody or antigen-binding fragment thereof comprises:
(a) an immunoglobulin heavy chain, comprising an amino acid sequence having at least 80%, 85%, 90%, 95%, 96%, 97%, 98%, 99% identity to the amino acid sequence set forth in SEQ ID NO: 55, and
(b) an immunoglobulin light chain, comprising an amino acid sequence having at least 80%, 85%, 90%, 95%, 96%, 97%, 98%, 99% identity to the amino acid sequence set forth in SEQ ID NO: 57.

31. An antibody or antigen-binding fragment thereof that specifically binds to canine programmed death protein 1 (cPD-1), wherein the antibody or antigen-binding fragment thereof comprises:
(a) an immunoglobulin heavy chain, comprising the amino acid sequence set forth in SEQ ID NO: 55, and
(b) an immunoglobulin light chain, comprising the amino acid sequence set forth in SEQ ID NO: 57.

32. The antibody or antigen-binding fragment thereof of claim 1, wherein the antibody or antigen-binding fragment thereof is an IgG, an IgM, an IgE, an IgA, or an IgD molecule, or is derived from one of these.

33. The antibody or antigen-binding fragment thereof of claim 1, wherein the antibody or antigen-binding fragment thereof is selected from the group consisting of a full-length antibody, a Fab, a single-chain variable fragment (scFv), sc(Fv)2, dsFv, Fab, Fab′, (Fab′)2 and a diabody.

34. The antibody or antigen-binding fragment thereof of claim 1, wherein the antibody is a full-length antibody.

35. The antibody or antigen-binding fragment thereof of claim 1, wherein the antibody is a canine or caninized antibody.

36. The antibody or antigen-binding fragment thereof of claim 1, wherein the antibody is a canine antibody.

37. The antibody or antigen-binding fragment thereof of claim 1, wherein the antigen-binding fragment is an scFv.

38. The antibody or antigen-binding fragment thereof of claim 1, wherein the specific binding of the antibody or antigen-binding fragment thereof to cPD-1 disrupts the interaction of cPD-1 and a ligand of cPD-1 (e.g., canine programmed death-ligand 1).

39. A canine antibody or antigen-binding fragment thereof that specifically binds to canine programmed death protein 1 (cPD-1), comprising the antibody or antigen-binding fragment thereof of claim 1.

40. (canceled)

41. The canine antibody or antigen-binding fragment thereof of claim 39, wherein the specific binding of the antibody or antigen-binding fragment thereof to cPD-1 disrupts the interaction of cPD-1 and a ligand of cPD-1 (e.g., canine programmed death-ligand 1).

42. A single-chain variable fragment (scFv) specifically binds canine programmed death protein 1 (cPD-1), comprising:
(a) a heavy chain variable region that comprises heavy chain complementarity determining region 1 (HCDR1), HCDR2, and HCDR3, wherein HCDR1 comprises an amino acid sequence selected from the group consisting of SEQ ID NOs: 1, 27, and 43, HCDR2 comprises an amino acid sequence selected from the group consisting of SEQ ID NOs: 2, 28, and 44, and HCDR3 comprises an amino acid sequence selected from the group consisting of SEQ ID NOs: 3, 29, and 45; and
(b) a light chain variable region that comprises light chain complementarity determining region 1 (LCDR1), LCDR2, and LCDR3, wherein LCDR1 comprises an amino acid sequence selected from the group consisting of SEQ ID NOs: 4, 30, and 46, LCDR2 comprises an amino acid sequence selected from the group consisting of SEQ ID NOs: 5, 31 and 47, and LCDR3 comprises an amino acid sequence selected from the group consisting of SEQ ID NOs: 6, 32, and 48,
wherein the heavy chain variable region and the light chain variable region are connected by a linker.

43. A single-chain variable fragment (scFv) specifically binds to canine programmed death protein 1 (cPD-1), comprising:
(a) a heavy chain variable region comprising the amino acid sequence set forth in SEQ ID NOs: 7, 9, 33, or 49; and
(b) a light chain variable region comprising the amino acid sequence set forth in SEQ ID NOs: 11, 35, or 51,
wherein the heavy chain variable region and the light chain variable region are connected by a linker.

44. A single chain variable fragment (scFv) specifically binds to canine programmed death protein 1 (cPD-1), comprising the amino acid sequence set forth in SEQ ID NOs: 13, 15, 17, 37, or 53.

45. The scFv of claim 42, wherein the specific binding of the antibody or antigen-binding fragment thereof to cPD-1 disrupts the interaction of cPD-1 and a ligand of cPD-1 (e.g., canine programmed death-ligand 1).

46. A bispecific molecule comprising the antibody or antigen-binding fragment thereof of claim 1, linked to a molecule having a second binding specificity.

47. An immunoconjugate comprising the antibody or antigen-binding fragment thereof of claim 1, linked to a therapeutic agent.

48. An isolated nucleic acid encoding the antibody or antigen-binding fragment thereof of claim 1.

49. An isolated nucleic acid encoding an antibody or antigen-binding fragment thereof, wherein the antibody or antigen-binding fragment thereof comprises:
(a) a heavy chain variable region encoded by a nucleic acid comprising a polynucleotide sequence having at least 80%, 85%, 90%, 95%, 96%, 96%, 97%, 98%, 99% identity to the polynucleotide sequence set forth in SEQ ID NOs: 8, 10, 34, or 50; and
(b) a light chain variable region encoded by a nucleic acid comprising a polynucleotide sequence having at least 80%, 85%, 90%, 95%, 96%, 96%, 97%, 98%, 99% identity to the polynucleotide sequence set forth in SEQ ID NOs: 12, 36, or 52;

50. The isolated nucleic acid of claim 49, wherein the heavy chain variable region is encoded by a nucleic acid comprising the polynucleotide sequence set forth in SEQ ID NOs: 8, 10, 34, or 50.

51. The isolated nucleic acid of claim 49 or 50, wherein the light chain variable region is encoded by a nucleic acid comprising the polynucleotide sequence set forth in SEQ ID NOs: 12, 36, or 52.

52. An isolated nucleic acid encoding an antibody or antigen binding fragment thereof comprising:
(a) a heavy chain variable region encoded by a nucleic acid sequence comprising the polynucleotide sequence set forth in SEQ ID NO: 8; and a light chain variable region encoded by a nucleic acid sequence comprising the polynucleotide sequence set forth in SEQ ID NO: 12;
(b) a heavy chain variable region encoded by a nucleic acid sequence comprising the polynucleotide sequence set forth in SEQ ID NO: 10; and a light chain variable region encoded by a nucleic acid sequence comprising the polynucleotide sequence set forth in SEQ ID NO: 12;
(c) a heavy chain variable region encoded by a nucleic acid sequence comprising the polynucleotide sequence set forth in SEQ ID NO: 34 and a light chain variable region encoded by a nucleic acid sequence comprising the polynucleotide sequence set forth in SEQ ID NO: 36; or
(d) a heavy chain variable region encoded by a nucleic acid sequence comprising the polynucleotide sequence set forth in SEQ ID NO: 50 and a light chain variable region encoded by a nucleic acid sequence comprising the polynucleotide sequence set forth in SEQ ID NO: 52.

53.-55. (canceled)

56. The isolated nucleic acid of claim 49, wherein the antibody or antigen-binding fragment thereof is an IgG, an IgM, an IgE, an IgA, or an IgD molecule, or is derived from one of these.

57. The isolated nucleic acid of claim 49, wherein the antibody or antigen-binding fragment thereof is selected from the group consisting of a full-length antibody, a Fab, a single-chain variable fragment (scFv), sc(Fv)2, dsFv, Fab, Fab′, (Fab′)2 and a diabody.

58. The isolated nucleic acid of claim 49, wherein the antibody is a full-length antibody.

59. The isolated nucleic acid of claim 49, wherein the antibody is a canine or caninized antibody.

60. The isolated nucleic acid of claim 49, wherein the antibody is a canine antibody.

61. An isolated nucleic acid encoding a single-chain variable fragment (scFv) comprising:
(a) a heavy chain variable region comprising the nucleotide sequence set forth in SEQ ID NOs: 8, 10, 34, or 50; and
(b) a light chain variable region comprising the nucleotide sequence set forth in SEQ ID NOs: 12, 36, or 52,
wherein the heavy chain variable region and the light chain variable region are connected by a linker.

62. An isolated nucleic acid encoding a single-chain variable fragment (scFv) comprising the polynucleotide sequence set forth in SEQ ID NOs: 14, 16, 18, 38, or 54.

63. The isolated nucleic acid of claim 61, wherein the antibody or antigen-binding fragment thereof or the scFv specifically binds to canine programmed death protein 1 (cPD-1).

64. The isolated nucleic acid of claim 63, wherein the specific binding of the antibody or antigen-binding fragment thereof or the scFv to cPD-1 disrupts the interaction of cPD-1 and a ligand of cPD-1 (e.g., canine programmed death-ligand 1).

65. A vector comprising the isolated nucleic acid of claim 49.

66. The vector of claim 65, wherein the vector is an expression vector.

67. The vector of claim 65, wherein the vector is selected from the group consisting of a DNA vector, an RNA vector, a plasmid, a lentiviral vector, an adenoviral vector, an adeno-associated viral vector, and a retroviral vector.

68. A host cell comprising the isolated nucleic acid of claim 49.

69. The host cell of claim 68, wherein the host cell is of eukaryotic or prokaryotic origin.

70. The host cell of claim 68, wherein the host cell is of mammalian origin.

71. The host cell of claim 68, wherein the host cell is of bacterial origin.

72. The host cell of claim 68, wherein the host cell is a Chinese Hamster Ovary cell.

73. A pharmaceutical composition comprising the antibody or antigen-binding fragment thereof of claim 1, and a pharmaceutically acceptable excipient, carrier, or diluent.

74. The pharmaceutical composition of claim 73, formulated for intravenous administration.

75. The pharmaceutical composition of claim 73, formulated for subcutaneous administration.

76. The pharmaceutical composition of claim 73, formulated for intratumoral administration.

77. A kit comprising the antibody or antigen-binding fragment thereof of claim 1, and an instruction for use.

78. A method for increasing T cell proliferation in a subject in need thereof, comprising administering to the subject the antibody or antigen-binding fragment thereof of claim 1.

79. A method for increasing IFNγ production in a subject in need thereof, comprising administering to the subject the antibody or antigen-binding fragment thereof of claim 1.

80. A method for inhibiting canine programmed death protein 1 (cPD-1) signaling pathway in a subject in need thereof, comprising administering to the subject the antibody or antigen-binding fragment thereof of claim 1.

81. The method of claim 78, wherein the subject has cancer or a chronic infection (e.g., viral infections).

82. A method for treating a disease or condition in a subject in need thereof, comprising administering to the subject the antibody or antigen-binding fragment thereof of claim 1.

83. The method of claim 82, wherein the disease or condition comprises cancer or a chronic infection (e.g., viral infections).

84. The method of claim 82, further comprising administering one or more additional therapeutics (e.g., chemotherapy) or interventions.

85. The method of claim 83, wherein the cancer is associated with canine programmed death protein 1 (cPD-1) signaling pathway.

86. The method of claim 85, wherein a ligand for cPD-1 (e.g., canine programmed death-ligand 1) is expressed on a cancer cell of the subject.

87. The method of claim 85, wherein cPD-1 is expressed on an immune cell of the subject.

88. The method of claim 87, wherein the immune cell is a tumor-infiltrating lymphocyte.

89. The method of any one of claim 83, wherein the cancer comprises a melanoma (e.g., metastatic malignant melanoma), a prostate cancer (for example hormone refractory prostate adenocarcinoma), a head and neck cancer (for example, squamous cell carcinoma of the head and neck), a cervical cancer, a thyroid cancer, a glioblastoma, a glioma, leukemia, a lymphoma (for example, a B cell lymphoma), an adrenal gland cancer, an AIDS-associated cancer, an alveolar soft part sarcoma, an astrocytic tumor, bone cancer, a brain and spinal cord cancer, a metastatic brain tumor, a carotid body tumor, a chondrosarcoma, a chordoma, a chromophobe renal cell carcinoma, a clear cell carcinoma, cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gestational trophoblastic disease, a germ cell tumor, a hematological malignancy, hepatocellular carcinoma, an islet cell tumor, a Kaposi's sarcoma, a kidney cancer, a lipoma/benign lipomatous tumor, a liposarcoma/malignant lipomatous tumor, a medulloblastoma, a meningioma, a Merkel cell carcinoma, a multiple endocrine neoplasia, a multiple myeloma, a myelodysplasia syndrome, a neuroblastoma, a neuroendocrine tumor, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomysarcoma, a sarcoma, a soft-tissue sarcoma, a squamous cell cancer, a stomach cancer, a synovial sarcoma, a testicular cancer, a thymic carcinoma, a thymoma, a thyroid metastatic cancer, a uterine cancer, or any combination thereof.

90. The method of any one of claim 83, wherein the cancer comprises cervical cancer, lung cancers, liver cancers, ovarian cancers, cancers of the skin including melanoma and squamous cell carcinoma, colon cancer, bladder cancer, breast cancer, kidney cancer, esophageal cancer, stomach cancer, pancreatic cancers, head cancer, and neck cancer.

91. The method of any one of claim 82, wherein the antibody or antigen-binding fragment thereof, the canine antibody, the scFv, the bispecific molecule, or the immunoconjugate specifically binds to cPD-1.

92. The method of claim 91, wherein the binding of antibody or antigen-binding fragment thereof, the canine antibody, the scFv, the bispecific molecule, or the immunoconjugate to cPD-1 disrupts the interaction of cPD-1 and a ligand of cPD-1 (e.g., canine programmed death-ligand 1).

93. The method of claim 82, wherein the subject is canine.

94. A method of making the pharmaceutical composition of claim of claim 73, comprising admixing the antibody or antigen-binding fragment thereof of claim 1, and a pharmaceutically acceptable excipient, carrier, or diluent.

95. A method of making a composition for treating cancer or chronic infection (e.g., viral infections), comprising admixing the antibody or antigen-binding fragment thereof of claim 1, and a pharmaceutically acceptable excipient, carrier, or diluent.

96. The method of claim 95, wherein the cancer comprises a melanoma (e.g., metastatic malignant melanoma), a prostate cancer (for example hormone refractory prostate adenocarcinoma), a head and neck cancer (for example, squamous cell carcinoma of the head and neck), a cervical cancer, a thyroid cancer, a glioblastoma, a glioma, leukemia, a lymphoma (for example, a B cell lymphoma), an adrenal gland cancer, an AIDS-associated cancer, an alveolar soft part sarcoma, an astrocytic tumor, bone cancer, a brain and spinal cord cancer, a metastatic brain tumor, a carotid body tumor, a chondrosarcoma, a chordoma, a chromophobe renal cell carcinoma, a clear cell carcinoma, cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, a gestational trophoblastic disease, a germ cell tumor, a hematological malignancy, hepatocellular carcinoma, an islet cell tumor, a Kaposi's sarcoma, a kidney cancer, a lipoma/benign lipomatous tumor, a liposarcoma/malignant lipomatous tumor, a medulloblastoma, a meningioma, a Merkel cell carcinoma, a multiple endocrine neoplasia, a multiple myeloma, a myelodysplasia syndrome, a neuroblastoma, a neuroendocrine tumor, a papillary thyroid carcinoma, a parathyroid tumor, a pediatric cancer, a peripheral nerve sheath tumor, a phaeochromocytoma, a pituitary tumor, a prostate cancer, a posterior uveal melanoma, a rare hematologic disorder, a renal metastatic cancer, a rhabdoid tumor, a rhabdomysarcoma, a sarcoma, a soft-tissue sarcoma, a squamous cell cancer, a stomach cancer, a synovial sarcoma, a testicular cancer, a thymic carcinoma, a thymoma, a thyroid metastatic cancer, a uterine cancer, or any combination thereof.

97. The method of claim 95, wherein the cancer comprises cervical cancer, lung cancers, liver cancers, ovarian cancers, cancers of the skin including melanoma and squamous cell carcinoma, colon cancer, bladder cancer, breast cancer, kidney cancer, esophageal cancer, stomach cancer, pancreatic cancers, head cancer, and neck cancer.

98.-101. (canceled)

102. A method of producing the antibody or antigen-binding fragment thereof of claim 1, the method comprising culturing a host cell comprising an isolated nucleic acid encoding said antibody or antigen-binding fragment thereof.

103. The method of claim 102, further comprising incubating the host cell in a cell culture medium under conditions sufficient to allow expression and secretion of the antibody or antigen-binding fragment thereof.


SEQUENCE LISTING (81 sequences)

SEQ ID NO: 1 - protein, 8 aa, synthetic construct
GFTFSKYG

SEQ ID NO: 2 - protein, 8 aa, synthetic construct
IAANGGTY

SEQ ID NO: 3 - protein, 9 aa, synthetic construct
RWGGAPFDY

SEQ ID NO: 4 - protein, 6 aa, synthetic construct
QSVSSY

SEQ ID NO: 5 - skipped

SEQ ID NO: 6 - protein, 9 aa, synthetic construct
QQGLQYPIT

SEQ ID NO: 7 - protein, 116 aa, synthetic construct
EVQLVQSGGDVVKPGGSLRLSCMASGFTFSKYGMSWVRQSPGKGLQWVADIAANGGTYYTDAVKGRFTISRDNAKNTLYLQMNSLRAEDTAVYYCARWGGAPFDYWGQGTLVTVSS

SEQ ID NO: 8 - DNA, 348 bp, synthetic construct
  source 1..348: mol_type = other DNA
gaggtgcagctggtgcagagcggaggagacgtggtgaagcctggaggaagcctgaggctgagctgcatggcaagcggattcaccttcagcaagtacggaatgagctgggtgaggcagagccccggcaagggcctgcagtgggtggccgacatcgccgccaacggcggcacctactacaccgacgccgtgaagggcaggttcaccatcagcagggacaacgccaagaacaccctgtacctgcagatgaacagcctgagggccgaggacaccgccgtgtactactgcgccaggtggggcggcgcccccttcgactactggggccagggcaccctggtgaccgtgagcagc

SEQ ID NO: 9 - protein, 116 aa, synthetic construct
EVQLVQSGGDVVKPGGSLRLSCVASGFTFSKYGMSWVRQSPGKGLQWVADIAANGGTYYTDAVKGRFTISRDNAKNTLYLQMNSLRAEDTAVYYCARWGGAPFDYWGQGTLVTVSS

SEQ ID NO: 10 - DNA, 348 bp, synthetic construct
  source 1..348: mol_type = other DNA
gaggtgcagctggtgcagagcggcggcgacgtggtgaagcccggcggcagcctgaggctgagctgcgtggccagcggcttcaccttcagcaagtacggcatgagctgggtgaggcagagccccggcaagggcctgcagtgggtggccgacatcgccgccaacggcggcacctactacaccgacgccgtgaagggcaggttcaccatcagcagggacaacgccaagaacaccctgtacctgcagatgaacagcctgagggccgaggacaccgccgtgtactactgcgccaggtggggcggcgcccccttcgactactggggccagggcaccctggtgactgtgagcagc

SEQ ID NO: 11 - protein, 107 aa, synthetic construct
EIVMTQSPASLSLSQEEKVTITCRASQSVSSYLAWYQQKPGQAPKLLIYGTSNRATGVPSRFSGSGSGTDFSFTISSLEPEDVAVYYCQQGLQYPITFGKGTHLEIK

SEQ ID NO: 12 - DNA, 321 bp, synthetic construct
  source 1..321: mol_type = other DNA
gaaatcgtgatgacccagtctccagcctccctctccttgtctcaggaggaaaaagtcaccatcacctgccgggccagtcagagtgttagcagctacttagcctggtaccagcaaaaacctgggcaggctcccaagctcctcatctatggtacatccaacagggccactggtgtcccatcccggttcagtggcagtgggtctgggacagacttcagcttcaccatcagcagcctggagcctgaagatgttgcagtttattactgccagcaaggtctacagtatccgatcacctttggcaaagggacacatctggagattaaa

SEQ ID NO: 13 - protein, 241 aa, synthetic construct
EIVMTQSPASLSLSQEEKVTITCRASQSVSSYLAWYQQKPGQAPKLLIYGTSNRATGVPSRFSGSGSGTDFSFTISSLEPEDVAVYYCQQGLQYPITFGKGTHLEIKGGSSRSSSSGGGGSGGGGEVQLVQSGGDLVKPGGSLRLSCMASGFTFSKYGMSWVRQSPGKGLQWVADIAANGGTYYTDAVKGRFTISRDNAKNALYLQMDDLRAEDTAVYYCARWGGAPFDYWGQGTLVTVSS

SEQ ID NO: 14 - DNA, 723 bp, synthetic construct
  source 1..723: mol_type = other DNA
gaaatcgtgatgacccagtctccagcctccctctccttgtctcaggaggaaaaagtcaccatcacctgccgggccagtcagagtgttagcagctacttagcctggtaccagcaaaaacctgggcaggctcccaagctcctcatctatggtacatccaacagggccactggtgtcccatcccggttcagtggcagtgggtctgggacagacttcagcttcaccatcagcagcctggagcctgaagatgttgcagtttattactgccagcaaggtctacagtatccgatcacctttggcaaagggacacatctggagattaaaggtggttcctctagatcttcctcctctggtggcggtggctcgggcggtggtggggaggtccagctggtgcagtctgggggagacctggtgaagcctggggggtccttgagactgtcctgtatggcctctggattcaccttcagtaaatatggcatgagctgggtccgtcagtctccagggaaggggctgcagtgggtcgcagatattgccgctaatggaggcacatactacacggacgctgtgaagggccgattcaccatctccagagacaacgccaagaacgcgctatatctgcagatggacgatctgagagccgaggacacggctgtctattattgtgcgaggtgggggggggccccctttgactactggggccagggaaccctggtcaccgtctcctca

SEQ ID NO: 15 - protein, 241 aa, synthetic construct
EIVMTQSPASLSLSQEEKVTITCRASQSVSSYLAWYQQKPGQAPKLLIYGTSNRATGVPSRFSGSGSGTDFSFTISSLEPEDVAVYYCQQGLQYPITFGKGTHLEIKGGSSRSSSSGGGGSGGGGEVQLVQSGGDVVKPGGSLRLSCMASGFTFSKYGMSWVRQSPGKGLQWVADIAANGGTYYTDAVKGRFTISRDNAKNTLYLQMNSLRAEDTAVYYCARWGGAPFDYWGQGTLVTVSS

SEQ ID NO: 16 - DNA, 723 bp, synthetic construct
  source 1..723: mol_type = other DNA
gaaatcgtgatgacccagtctccagcctccctctccttgtctcaggaggaaaaagtcaccatcacctgccgggccagtcagagtgttagcagctacttagcctggtaccagcaaaaacctgggcaggctcccaagctcctcatctatggtacatccaacagggccactggtgtcccatcccggttcagtggcagtgggtctgggacagacttcagcttcaccatcagcagcctggagcctgaagatgttgcagtttattactgccagcaaggtctacagtatccgatcacctttggcaaagggacacatctggagattaaaggtggttcctctagatcttcctcctctggtggcggtggctcgggcggtggtggggaggtgcagctggtgcagagcggaggagacgtggtgaagcctggaggaagcctgaggctgagctgcatggcaagcggattcaccttcagcaagtacggaatgagctgggtgaggcagagccccggcaagggcctgcagtgggtggccgacatcgccgccaacggcggcacctactacaccgacgccgtgaagggcaggttcaccatcagcagggacaacgccaagaacaccctgtacctgcagatgaacagcctgagggccgaggacaccgccgtgtactactgcgccaggtggggcggcgcccccttcgactactggggccagggcaccctggtgaccgtgagcagc

SEQ ID NO: 17 - protein, 241 aa, synthetic construct
EIVMTQSPASLSLSQEEKVTITCRASQSVSSYLAWYQQKPGQAPKLLIYGTSNRATGVPSRFSGSGSGTDFSFTISSLEPEDVAVYYCQQGLQYPITFGKGTHLEIKGGSSRSSSSGGGGSGGGGEVQLVQSGGDVVKPGGSLRLSCVASGFTFSKYGMSWVRQSPGKGLQWVADIAANGGTYYTDAVKGRFTISRDNAKNTLYLQMNSLRAEDTAVYYCARWGGAPFDYWGQGTLVTVSS

SEQ ID NO: 18 - DNA, 723 bp, synthetic construct
  source 1..723: mol_type = other DNA
gaaatcgtgatgacccagtctccagcctccctctccttgtctcaggaggaaaaagtcaccatcacctgccgggccagtcagagtgttagcagctacttagcctggtaccagcaaaaacctgggcaggctcccaagctcctcatctatggtacatccaacagggccactggtgtcccatcccggttcagtggcagtgggtctgggacagacttcagcttcaccatcagcagcctggagcctgaagatgttgcagtttattactgccagcaaggtctacagtatccgatcacctttggcaaagggacacatctggagattaaaggtggttcctctagatcttcctcctctggtggcggtggctcgggcggtggtggggaggtgcagctggtgcagagcggcggcgacgtggtgaagcccggcggcagcctgaggctgagctgcgtggccagcggcttcaccttcagcaagtacggcatgagctgggtgaggcagagccccggcaagggcctgcagtgggtggccgacatcgccgccaacggcggcacctactacaccgacgccgtgaagggcaggttcaccatcagcagggacaacgccaagaacaccctgtacctgcagatgaacagcctgagggccgaggacaccgccgtgtactactgcgccaggtggggcggcgcccccttcgactactggggccagggcaccctggtgactgtgagcagc

SEQ ID NO: 19 - protein, 447 aa, synthetic construct
EVQLVQSGGDVVKPGGSLRLSCMASGFTFSKYGMSWVRQSPGKGLQWVADIAANGGTYYTDAVKGRFTISRDNAKNTLYLQMNSLRAEDTAVYYCARWGGAPFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 20 - DNA, 1341 bp, synthetic construct
  source 1..1341: mol_type = other DNA
gaggtgcagctggtgcagagcggaggagacgtggtgaagcctggaggaagcctgaggctgagctgcatggcaagcggattcaccttcagcaagtacggaatgagctgggtgaggcagagccccggcaagggcctgcagtgggtggccgacatcgccgccaacggcggcacctactacaccgacgccgtgaagggcaggttcaccatcagcagggacaacgccaagaacaccctgtacctgcagatgaacagcctgagggccgaggacaccgccgtgtactactgcgccaggtggggcggcgcccccttcgactactggggccagggcaccctggtgaccgtgagcagcgctagcaccacggccccctcggttttcccactggcccccagctgcgggtccacttccggctccacggtggccctagcctgcctggtgtcaggctacttccccgagcctgtaactgtgtcctggaactccggctccttgaccagcggtgtgcacaccttcccgtccgtcctgcagtcctcagggctctactccctcagcagcatggtgacagtgccctccagcaggtggcccagcgagaccttcacctgcaacgtggtccacccggccagcaacactaaagtagacaagccagtgcccaaagagtccacctgcaagtgtatatccccatgcccagtccctgaatcactgggagggccttcggtcttcatctttcccccgaaacccaaggacatcctcaggattacccgaacacccgagatcacctgtgtggtgttagatctgggccgtgaggaccctgaggtgcagatcagctggttcgtggatggtaaggaggtgcacacagccaagacgcagcctcgtgagcagcagttcaacagcacctaccgtgtggtcagcgtcctccccattgagcaccaggactggctcaccggaaaggagttcaagtgcagagtcaaccacataggcctcccgtcccccatcgagaggaccatctccaaagccagagggcaagcccatcagcccagtgtgtatgtcctgccaccatccccaaaggagttgtcatccagtgacacggtcaccctgacctgcctgatcaaagacttcttcccacctgagattgatgtggagtggcagagcaatggacagccagagcctgagagcaagtaccacacgactgcaccccagctggacgaggacgggtcctacttcctgtacagcaagctctctgtggacaagagccgctggcagcagggagaccccttcacatgtgcggtgatgcatgaagctctacagaaccactacacagatctatccctctcccattctccgggtaaa

SEQ ID NO: 21 - protein, 447 aa, synthetic construct
EVQLVQSGGDVVKPGGSLRLSCVASGFTFSKYGMSWVRQSPGKGLQWVADIAANGGTYYTDAVKGRFTISRDNAKNTLYLQMNSLRAEDTAVYYCARWGGAPFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 22 - DNA, 1341 bp, synthetic construct
  source 1..1341: mol_type = other DNA
gaggtgcagctggtgcagagcggcggcgacgtggtgaagcccggcggcagcctgaggctgagctgcgtggccagcggcttcaccttcagcaagtacggcatgagctgggtgaggcagagccccggcaagggcctgcagtgggtggccgacatcgccgccaacggcggcacctactacaccgacgccgtgaagggcaggttcaccatcagcagggacaacgccaagaacaccctgtacctgcagatgaacagcctgagggccgaggacaccgccgtgtactactgcgccaggtggggcggcgcccccttcgactactggggccagggcaccctggtgactgtgagcagcgctagcaccacggccccctcggttttcccactggcccccagctgcgggtccacttccggctccacggtggccctagcctgcctggtgtcaggctacttccccgagcctgtaactgtgtcctggaactccggctccttgaccagcggtgtgcacaccttcccgtccgtcctgcagtcctcagggctctactccctcagcagcatggtgacagtgccctccagcaggtggcccagcgagaccttcacctgcaacgtggtccacccggccagcaacactaaagtagacaagccagtgcccaaagagtccacctgcaagtgtatatccccatgcccagtccctgaatcactgggagggccttcggtcttcatctttcccccgaaacccaaggacatcctcaggattacccgaacacccgagatcacctgtgtggtgttagatctgggccgtgaggaccctgaggtgcagatcagctggttcgtggatggtaaggaggtgcacacagccaagacgcagcctcgtgagcagcagttcaacagcacctaccgtgtggtcagcgtcctccccattgagcaccaggactggctcaccggaaaggagttcaagtgcagagtcaaccacataggcctcccgtcccccatcgagaggaccatctccaaagccagagggcaagcccatcagcccagtgtgtatgtcctgccaccatccccaaaggagttgtcatccagtgacacggtcaccctgacctgcctgatcaaagacttcttcccacctgagattgatgtggagtggcagagcaatggacagccagagcctgagagcaagtaccacacgactgcaccccagctggacgaggacgggtcctacttcctgtacagcaagctctctgtggacaagagccgctggcagcagggagaccccttcacatgtgcggtgatgcatgaagctctacagaaccactacacagatctatccctctcccattctccgggtaaa

SEQ ID NO: 23 - protein, 447 aa, synthetic construct
EVQLVQSGGDLVKPGGSLRLSCMASGFTFSKYGMSWVRQSPGKGLQWVADIAANGGTYYTDAVKGRFTISRDNAKNALYLQMDDLRAEDTAVYYCARWGGAPFDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 24 - DNA, 1341 bp, synthetic construct
  source 1..1341: mol_type = other DNA
gaggtccagctggtgcagtctgggggagacctggtgaagcctggggggtccttgagactgtcctgtatggcctctggattcaccttcagtaaatatggcatgagctgggtccgtcagtctccagggaaggggctgcagtgggtcgcagatattgccgctaatggaggcacatactacacggacgctgtgaagggccgattcaccatctccagagacaacgccaagaacgcgctatatctgcagatggacgatctgagagccgaggacacggctgtctattattgtgcgaggtgggggggggccccctttgactactggggccagggaaccctggtcaccgtctcctcagctagcaccacggccccctcggttttcccactggcccccagctgcgggtccacttccggctccacggtggccctagcctgcctggtgtcaggctacttccccgagcctgtaactgtgtcctggaactccggctccttgaccagcggtgtgcacaccttcccgtccgtcctgcagtcctcagggctctactccctcagcagcatggtgacagtgccctccagcaggtggcccagcgagaccttcacctgcaacgtggtccacccggccagcaacactaaagtagacaagccagtgcccaaagagtccacctgcaagtgtatatccccatgcccagtccctgaatcactgggagggccttcggtcttcatctttcccccgaaacccaaggacatcctcaggattacccgaacacccgagatcacctgtgtggtgttagatctgggccgtgaggaccctgaggtgcagatcagctggttcgtggatggtaaggaggtgcacacagccaagacgcagcctcgtgagcagcagttcaacagcacctaccgtgtggtcagcgtcctccccattgagcaccaggactggctcaccggaaaggagttcaagtgcagagtcaaccacataggcctcccgtcccccatcgagaggaccatctccaaagccagagggcaagcccatcagcccagtgtgtatgtcctgccaccatccccaaaggagttgtcatccagtgacacggtcaccctgacctgcctgatcaaagacttcttcccacctgagattgatgtggagtggcagagcaatggacagccagagcctgagagcaagtaccacacgactgcaccccagctggacgaggacgggtcctacttcctgtacagcaagctctctgtggacaagagccgctggcagcagggagaccccttcacatgtgcggtgatgcatgaagctctacagaaccactacacagatctatccctctcccattctccgggtaaa

SEQ ID NO: 25 - protein, 217 aa, synthetic construct
EIVMTQSPASLSLSQEEKVTITCRASQSVSSYLAWYQQKPGQAPKLLIYGTSNRATGVPSRFSGSGSGTDFSFTISSLEPEDVAVYYCQQGLQYPITFGKGTHLEIKRTDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 26 - DNA, 651 bp, synthetic construct
  source 1..651: mol_type = other DNA
gaaatcgtgatgacccagtctccagcctccctctccttgtctcaggaggaaaaagtcaccatcacctgccgggccagtcagagtgttagcagctacttagcctggtaccagcaaaaacctgggcaggctcccaagctcctcatctatggtacatccaacagggccactggtgtcccatcccggttcagtggcagtgggtctgggacagacttcagcttcaccatcagcagcctggagcctgaagatgttgcagtttattactgccagcaaggtctacagtatccgatcacctttggcaaagggacacatctggagattaaacgtacggatgcccagccagccgtctatttgttccaaccatctccagaccagttacacacaggaagtgcctctgttgtgtgtttgctgaatagcttctaccccaaagacatcaatgtcaagtggaaagtggatggtgtcatccaagacacaggcatccaggaaagtgtcacagagcaggacaaggacagtacctacagcctcagcagcaccctgacgatgtccagtactgagtacctaagtcatgagttgtactcctgtgagatcactcacaagagcctgccctccaccctcatcaagagcttccaaaggagcgagtgtcagagagtggac

SEQ ID NO: 27 - protein, 8 aa, synthetic construct
GFTFNTYG

SEQ ID NO: 28 - protein, 8 aa, synthetic construct
IANSGSTY

SEQ ID NO: 29 - protein, 10 aa, synthetic construct
SGIWGDWFYY

SEQ ID NO: 30 - protein, 6 aa, synthetic construct
SLTKYY

SEQ ID NO: 31 - skipped

SEQ ID NO: 32 - protein, 10 aa, synthetic construct
ESAVSADAVV

SEQ ID NO: 33 - protein, 117 aa, synthetic construct
EVQLVQSGGDVVKPGGSLRLSCVASGFTFNTYGMNWVRQSPGKGLQWVAAIANSGSTYYADAVRGRFTISRDNAKNTLYLQMNSLRAEDAAIYYCASGIWGDWFYYWGQGTLVTVSS

SEQ ID NO: 34 - DNA, 351 bp, synthetic construct
  source 1..351: mol_type = other DNA
gaggtgcagctggtgcagtctgggggagacgtggtgaagcctggggggtccttgagactgtcctgtgtggcctctggattcacctttaatacctatggcatgaactgggtccgtcagtctcctgggaaggggctgcagtgggtcgcggctattgccaatagtggaagcacatattacgcagacgctgtgaggggccggttcaccatctccagagacaacgccaagaacacactttatcttcagatgaacagcctgagagccgaggatgcggccatatattactgtgcgagtggtatttggggtgactggttctactactggggccaagggaccctggtcactgtgtcctca

SEQ ID NO: 35 - protein, 107 aa, synthetic construct
SSVLTQPPSVSVSLGQTATISCSGESLTKYYAQWFQQKPGQAPMLVIYKDTERPSGIPDRFSGSSSGNTHTLTISGARAEDEADYYCESAVSADAVVFGGGTHLTVL

SEQ ID NO: 36 - DNA, 321 bp, synthetic construct
  source 1..321: mol_type = other DNA
tccagtgtgctgactcagcctccctcggtatcagtgtctctgggacagacagcaaccatctcctgctctggagagagtctgactaaatattatgcacaatggttccagcagaagccaggccaagcccctatgttggtcatatataaggacactgagcggccctctgggatccctgaccgattctctggctccagttcaggaaacacacacaccctgaccatcagcggggctcgggccgaggacgaggccgactattactgcgagtcagcggtcagtgctgatgctgttgtgttcggcgggggcacccatctgaccgtcctc

SEQ ID NO: 37 - protein, 243 aa, synthetic construct
SSVLTQPPSVSVSLGQTATISCSGESLTKYYAQWFQQKPGQAPMLVIYKDTERPSGIPDRFSGSSSGNTHTLTISGARAEDEADYYCESAVSADAVVFGGGTHLTVLGGGSSRSSSSGGGGSGGGGEVQLVQSGGDVVKPGGSLRLSCVASGFTFNTYGMNWVRQSPGKGLQWVAAIANSGSTYYADAVRGRFTISRDNAKNTLYLQMNSLRAEDAAIYYCASGIWGDWFYYWGQGTLVTVSS

SEQ ID NO: 38 - DNA, 729 bp, synthetic construct
  source 1..729: mol_type = other DNA
tccagtgtgctgactcagcctccctcggtatcagtgtctctgggacagacagcaaccatctcctgctctggagagagtctgactaaatattatgcacaatggttccagcagaagccaggccaagcccctatgttggtcatatataaggacactgagcggccctctgggatccctgaccgattctctggctccagttcaggaaacacacacaccctgaccatcagcggggctcgggccgaggacgaggccgactattactgcgagtcagcggtcagtgctgatgctgttgtgttcggcgggggcacccatctgaccgtcctcggtggtggttcctctagatcttcctcctctggtggcggtggctcgggcggtggtggggaggtgcagctggtgcagtctgggggagacgtggtgaagcctggggggtccttgagactgtcctgtgtggcctctggattcacctttaatacctatggcatgaactgggtccgtcagtctcctgggaaggggctgcagtgggtcgcggctattgccaatagtggaagcacatattacgcagacgctgtgaggggccggttcaccatctccagagacaacgccaagaacacactttatcttcagatgaacagcctgagagccgaggatgcggccatatattactgtgcgagtggtatttggggtgactggttctactactggggccaagggaccctggtcactgtgtcctca

SEQ ID NO: 39 - protein, 448 aa, synthetic construct
EVQLVQSGGDVVKPGGSLRLSCVASGFTFNTYGMNWVRQSPGKGLQWVAAIANSGSTYYADAVRGRFTISRDNAKNTLYLQMNSLRAEDAAIYYCASGIWGDWFYYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 40 - DNA, 1344 bp, synthetic construct
  source 1..1344: mol_type = other DNA
gaggtgcagctggtgcagtctgggggagacgtggtgaagcctggggggtccttgagactgtcctgtgtggcctctggattcacctttaatacctatggcatgaactgggtccgtcagtctcctgggaaggggctgcagtgggtcgcggctattgccaatagtggaagcacatattacgcagacgctgtgaggggccggttcaccatctccagagacaacgccaagaacacactttatcttcagatgaacagcctgagagccgaggatgcggccatatattactgtgcgagtggtatttggggtgactggttctactactggggccaagggaccctggtcactgtgtcctcagctagcaccacggccccctcggttttcccactggcccccagctgcgggtccacttccggctccacggtggccctagcctgcctggtgtcaggctacttccccgagcctgtaactgtgtcctggaactccggctccttgaccagcggtgtgcacaccttcccgtccgtcctgcagtcctcagggctctactccctcagcagcatggtgacagtgccctccagcaggtggcccagcgagaccttcacctgcaacgtggtccacccggccagcaacactaaagtagacaagccagtgcccaaagagtccacctgcaagtgtatatccccatgcccagtccctgaatcactgggagggccttcggtcttcatctttcccccgaaacccaaggacatcctcaggattacccgaacacccgagatcacctgtgtggtgttagatctgggccgtgaggaccctgaggtgcagatcagctggttcgtggatggtaaggaggtgcacacagccaagacgcagcctcgtgagcagcagttcaacagcacctaccgtgtggtcagcgtcctccccattgagcaccaggactggctcaccggaaaggagttcaagtgcagagtcaaccacataggcctcccgtcccccatcgagaggaccatctccaaagccagagggcaagcccatcagcccagtgtgtatgtcctgccaccatccccaaaggagttgtcatccagtgacacggtcaccctgacctgcctgatcaaagacttcttcccacctgagattgatgtggagtggcagagcaatggacagccagagcctgagagcaagtaccacacgactgcaccccagctggacgaggacgggtcctacttcctgtacagcaagctctctgtggacaagagccgctggcagcagggagaccccttcacatgtgcggtgatgcatgaagctctacagaaccactacacagatctatccctctcccattctccgggtaaa

SEQ ID NO: 41 - protein, 213 aa, synthetic construct
SSVLTQPPSVSVSLGQTATISCSGESLTKYYAQWFQQKPGQAPMLVIYKDTERPSGIPDRFSGSSSGNTHTLTISGARAEDEADYYCESAVSADAVVFGGGTHLTVLGQPKASPSVTLFPPSSEELGANKATLVCLISDFYPSGVTVAWKADGSPITQGVETTKPSKQSNNKYAASSYLSLTPDKWKSHSSFSCLVTHEGSTVEKKVAPAECS

SEQ ID NO: 42 - DNA, 639 bp, synthetic construct
  source 1..639: mol_type = other DNA
tccagtgtgctgactcagcctccctcggtatcagtgtctctgggacagacagcaaccatctcctgctctggagagagtctgactaaatattatgcacaatggttccagcagaagccaggccaagcccctatgttggtcatatataaggacactgagcggccctctgggatccctgaccgattctctggctccagttcaggaaacacacacaccctgaccatcagcggggctcgggccgaggacgaggccgactattactgcgagtcagcggtcagtgctgatgctgttgtgttcggcgggggcacccatctgaccgtcctcggtcagcccaaggcctccccctcggtcacactcttcccgccctcctctgaggagctcggcgccaacaaggccaccctggtgtgcctcatcagcgacttctaccccagcggcgtgacggtggcctggaaggcagacggcagccccatcacccagggcgtggagaccaccaagccctccaagcagagcaacaacaagtacgcggccagcagctacctgagcctgacgcctgacaagtggaaatctcacagcagcttcagctgcctggtcacgcacgaggggagcaccgtggagaagaaggtggcccccgcagagtgctct

SEQ ID NO: 43 - protein, 8 aa, synthetic construct
GFTFSSYD

SEQ ID NO: 44 - protein, 8 aa, synthetic construct
ITTSGGST

SEQ ID NO: 45 - protein, 7 aa, synthetic construct
AVGALGD

SEQ ID NO: 46 - protein, 8 aa, synthetic construct
ASNIGLIG

SEQ ID NO: 47 - skipped

SEQ ID NO: 48 - protein, 12 aa, synthetic construct
QSIDSSLGDHFV

SEQ ID NO: 49 - protein, 114 aa, synthetic construct
EVQLVESGGDLVKPGGSLRLSCVASGFTFSSYDMNWVRQSPGKGLQWVAAITTSGGSTYYTDAVKGRFTISRDNAKNTLYLQMNNLRAEDTAVYYCAVGALGDWGQGTLVTVSS

SEQ ID NO: 50 - DNA, 342 bp, synthetic construct
  source 1..342: mol_type = other DNA
gaggtacagctggtggaatctgggggagacttggtgaagcctggggggtccttgagactgtcctgtgtggcctctggattcaccttcagtagctatgacatgaactgggtccgtcagtctccagggaaggggctgcagtgggtcgcagctattaccactagtggaggtagtacatactacacagacgctgtgaagggccgattcaccatctccagagacaatgccaagaacacactatatctgcagatgaacaacctgagagccgaggacacggctgtgtattattgtgcagtaggggctcttggtgactggggccagggcaccctggtcactgtctcctca

SEQ ID NO: 51 - protein, 111 aa, synthetic construct
QPVLTQPPSVSGALGQKVTISCSGSASNIGLIGAGWYQQVPGKAPKLLVYSDGNRPPQIPARFSGSNSGNSATLTIAGLQAEDEADYYCQSIDSSLGDHFVFGGGTHLTVL

SEQ ID NO: 52 - DNA, 333 bp, synthetic construct
  source 1..333: mol_type = other DNA
cagcctgtgctcactcagccgccctcagtgtcgggggcccttggccagaaggtcaccatctcctgctctggaagcgcgagcaacatcggtcttattggtgcgggctggtaccaacaagtcccaggaaaggcccctaaactcctcgtgtacagtgatgggaatcgaccgccacaaatccctgcccggttctccggctccaactctggcaactcagccaccctgactatcgctgggcttcaggctgaggacgaggctgattattactgtcagtctattgattccagccttggtgatcattttgtgttcggcggaggcacccatctgaccgtcctc

SEQ ID NO: 53 - protein, 244 aa, synthetic construct
QPVLTQPPSVSGALGQKVTISCSGSASNIGLIGAGWYQQVPGKAPKLLVYSDGNRPPQIPARFSGSNSGNSATLTIAGLQAEDEADYYCQSIDSSLGDHFVFGGGTHLTVLGGGSSRSSSSGGGGSGGGGEVQLVESGGDLVKPGGSLRLSCVASGFTFSSYDMNWVRQSPGKGLQWVAAITTSGGSTYYTDAVKGRFTISRDNAKNTLYLQMNNLRAEDTAVYYCAVGALGDWGQGTLVTVSS

SEQ ID NO: 54 - DNA, 732 bp, synthetic construct
  source 1..732: mol_type = other DNA
cagcctgtgctcactcagccgccctcagtgtcgggggcccttggccagaaggtcaccatctcctgctctggaagcgcgagcaacatcggtcttattggtgcgggctggtaccaacaagtcccaggaaaggcccctaaactcctcgtgtacagtgatgggaatcgaccgccacaaatccctgcccggttctccggctccaactctggcaactcagccaccctgactatcgctgggcttcaggctgaggacgaggctgattattactgtcagtctattgattccagccttggtgatcattttgtgttcggcggaggcacccatctgaccgtcctcggtggtggttcctctagatcttcctcctctggcggcggtggctcgggcggtggtggggaggtacagctggtggaatctgggggagacttggtgaagcctggggggtccttgagactgtcctgtgtggcctctggattcaccttcagtagctatgacatgaactgggtccgtcagtctccagggaaggggctgcagtgggtcgcagctattaccactagtggaggtagtacatactacacagacgctgtgaagggccgattcaccatctccagagacaatgccaagaacacactatatctgcagatgaacaacctgagagccgaggacacggctgtgtattattgtgcagtaggggctcttggtgactggggccagggcaccctggtcactgtctcctca

SEQ ID NO: 55 - protein, 445 aa, synthetic construct
EVQLVESGGDLVKPGGSLRLSCVASGFTFSSYDMNWVRQSPGKGLQWVAAITTSGGSTYYTDAVKGRFTISRDNAKNTLYLQMNNLRAEDTAVYYCAVGALGDWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDPFTCAVMHEALQNHYTDLSLSHSPGK

SEQ ID NO: 56 - DNA, 1335 bp, synthetic construct
  source 1..1335: mol_type = other DNA
gaggtacagctggtggaatctgggggagacttggtgaagcctggggggtccttgagactgtcctgtgtggcctctggattcaccttcagtagctatgacatgaactgggtccgtcagtctccagggaaggggctgcagtgggtcgcagctattaccactagtggaggtagtacatactacacagacgctgtgaagggccgattcaccatctccagagacaatgccaagaacacactatatctgcagatgaacaacctgagagccgaggacacggctgtgtattattgtgcagtaggggctcttggtgactggggccagggcaccctggtcactgtctcctcagctagcaccacggccccctcggttttcccactggcccccagctgcgggtccacttccggctccacggtggccctagcctgcctggtgtcaggctacttccccgagcctgtaactgtgtcctggaactccggctccttgaccagcggtgtgcacaccttcccgtccgtcctgcagtcctcagggctctactccctcagcagcatggtgacagtgccctccagcaggtggcccagcgagaccttcacctgcaacgtggtccacccggccagcaacactaaagtagacaagccagtgcccaaagagtccacctgcaagtgtatatccccatgcccagtccctgaatcactgggagggccttcggtcttcatctttcccccgaaacccaaggacatcctcaggattacccgaacacccgagatcacctgtgtggtgttagatctgggccgtgaggaccctgaggtgcagatcagctggttcgtggatggtaaggaggtgcacacagccaagacgcagcctcgtgagcagcagttcaacagcacctaccgtgtggtcagcgtcctccccattgagcaccaggactggctcaccggaaaggagttcaagtgcagagtcaaccacataggcctcccgtcccccatcgagaggaccatctccaaagccagagggcaagcccatcagcccagtgtgtatgtcctgccaccatccccaaaggagttgtcatccagtgacacggtcaccctgacctgcctgatcaaagacttcttcccacctgagattgatgtggagtggcagagcaatggacagccagagcctgagagcaagtaccacacgactgcaccccagctggacgaggacgggtcctacttcctgtacagcaagctctctgtggacaagagccgctggcagcagggagaccccttcacatgtgcggtgatgcatgaagctctacagaaccactacacagatctatccctctcccattctccgggtaaa

SEQ ID NO: 57 - protein, 217 aa, synthetic construct
QPVLTQPPSVSGALGQKVTISCSGSASNIGLIGAGWYQQVPGKAPKLLVYSDGNRPPQIPARFSGSNSGNSATLTIAGLQAEDEADYYCQSIDSSLGDHFVFGGGTHLTVLGQPKASPSVTLFPPSSEELGANKATLVCLISDFYPSGVTVAWKADGSPITQGVETTKPSKQSNNKYAASSYLSLTPDKWKSHSSFSCLVTHEGSTVEKKVAPAECS

SEQ ID NO: 58 - DNA, 651 bp, synthetic construct
  source 1..651: mol_type = other DNA
cagcctgtgctcactcagccgccctcagtgtcgggggcccttggccagaaggtcaccatctcctgctctggaagcgcgagcaacatcggtcttattggtgcgggctggtaccaacaagtcccaggaaaggcccctaaactcctcgtgtacagtgatgggaatcgaccgccacaaatccctgcccggttctccggctccaactctggcaactcagccaccctgactatcgctgggcttcaggctgaggacgaggctgattattactgtcagtctattgattccagccttggtgatcattttgtgttcggcggaggcacccatctgaccgtcctaggtcagcccaaggcctccccctcggtcacactcttcccgccctcctctgaggagctcggcgccaacaaggccaccctggtgtgcctcatcagcgacttctaccccagcggcgtgacggtggcctggaaggcagacggcagccccatcacccagggcgtggagaccaccaagccctccaagcagagcaacaacaagtacgcggccagcagctacctgagcctgacgcctgacaagtggaaatctcacagcagcttcagctgcctggtcacgcacgaggggagcaccgtggagaagaaggtggcccccgcagagtgctct

SEQ ID NO: 59 - DNA, 60 bp, synthetic construct
  source 1..60: mol_type = other DNA
atgtacaggatgcaactcctgtcttgcattgcactaagtcttgcacttgtcacgaattcg

SEQ ID NO: 60 - protein, 20 aa, synthetic construct
MYRMQLLSCIALSLALVTNS

SEQ ID NO: 61 - DNA, 51 bp, synthetic construct
  source 1..51: mol_type = other DNA
ggcggaggctccggaggcggatcttacccatacgatgttccagattacgct

SEQ ID NO: 62 - protein, 17 aa, synthetic construct
GGGSGGGSYPYDVPDYA

SEQ ID NO: 63 - DNA, 99 bp, synthetic construct
  source 1..99: mol_type = other DNA
gcctccaccacggccccctcggtcactagtggccaggccggccagcaccatcaccatcaccatggcgcatacccgtacgacgttccggactacgcttct

SEQ ID NO: 64 - protein, 33 aa, synthetic construct
ASTTAPSVTSGQAGQHHHHHHGAYPYDVPDYAS

SEQ ID NO: 65 - protein, 5 aa, synthetic construct
  REPEAT 1..5: note = n represents an integer of at least 1
GSGGS

SEQ ID NO: 66 - protein, 4 aa, synthetic construct
  REPEAT 1..4: note = n represents an integer of at least 1
GGGS

SEQ ID NO: 67 - protein, 5 aa, synthetic construct
  REPEAT 1..5: note = n represents an integer of at least 1
GGGGS

SEQ ID NO: 68 - protein, 4 aa, synthetic construct
GGSG

SEQ ID NO: 69 - protein, 5 aa, synthetic construct
GGSGG

SEQ ID NO: 70 - protein, 5 aa, synthetic construct
GSGSG

SEQ ID NO: 71 - protein, 5 aa, synthetic construct
GSGGG

SEQ ID NO: 72 - protein, 5 aa, synthetic construct
GGGSG

SEQ ID NO: 73 - protein, 5 aa, synthetic construct
GSSSG

SEQ ID NO: 74 - protein, 5 aa, synthetic construct
GGGGS

SEQ ID NO: 75 - protein, 15 aa, synthetic construct
GGGGSGGGGSGGGGS

SEQ ID NO: 76 - protein, 19 aa, synthetic construct
GGGSSRSSSSGGGGSGGGG

SEQ ID NO: 77 - protein, 57 aa, synthetic construct
GGCGGTGGTTCCTCTAGATCTTCCTCCTCTGGTGGCGGTGGCTCGGGCGGTGGTGGG

SEQ ID NO: 78 - protein, 7 aa, synthetic construct
AAEPKSS

SEQ ID NO: 79 - protein, 17 aa, synthetic construct
AAEPKSSDKTHTCPPCP

SEQ ID NO: 80 - protein, 4 aa, synthetic construct
GGGG

SEQ ID NO: 81 - protein, 14 aa, synthetic construct
GGGGDKTHTCPPCP